(19) United States
(12) Patent Application Publication
(10) Pub. No.: US 2022/0333073 A1
(43) Pub. Date: Oct. 20, 2022

(54) Compositions and Methods for Generating Gamma-Delta T Cells from Induced Pluripotent Stem Cells

(71) Applicant: Century Therapeutics, Inc., Philadelphia, PA (US)

(72) Inventors: Mark Wallet, Philadelphia, PA (US); Toshinobu Nishimura, Philadelphia, PA (US); Mark Mendonca, Philadelphia, PA (US); Andriana Lebid, Philadelphia, PA (US); Brenda Salantes, Philadelphia, PA (US); Katherine Santostefano, Philadelphia, PA (US); Michael Francis Naso, Philadelphia, PA (US); Buddha Gurung, Philadelphia, PA (US); Zengrong Zhu, Philadelphia, PA (US); Barry Morse, Philadelphia, PA (US); Luis Borges, Philadelphia, PA (US); Christina Del Casale, Philadelphia, PA (US); Marilda Beqiri, Philadelphia, PA (US); Lucas Thompson, Philadelphia, PA (US)

(21) Appl. No.: 17/657,798

(22) Filed: Apr. 4, 2022

Related U.S. Application Data
(60) Provisional application No. 63/171,646, filed on Apr. 7, 2021.
(60) Provisional application No. 63/279,837, filed on Nov. 16, 2021.

(51) Int. Cl. C12N 5/0783; C07K 14/725; C12N 5/074; C07K 16/28

(52) U.S. Cl. CPC C12N 5/0636; C07K 14/7051; C12N 5/0696; C07K 16/2887; C12N 2501/2302; C12N 2510/00; C07K 2319/03; C12N 2506/45

(57) ABSTRACT

Provided are methods for generating γδ T cells from induced pluripotent stem cells. Also provided are genetically engineered iPSCs, γδ T cells, CAR-γδ T cells, and methods of using the same.

### CROSS-REFERENCE TO RELATED APPLICATIONS

This application claims the benefit of U.S. Provisional Patent Application No. 63/171,646 filed Apr. 7, 2021, and U.S. Provisional Patent Application No. 63/279,837 filed Nov. 16, 2021, each of which is incorporated by reference herein in its entirety.

### TECHNICAL FIELD

This application provides genetically engineered induced pluripotent stem cells (iPSCs) and derivative cells thereof expressing a rearranged γδ T cell receptor (TCR). Also provided are uses of the iPSCs or derivative cells thereof to express a chimeric antigen receptor for allogeneic cell therapy. Further provided are related vectors, polynucleotides, and pharmaceutical compositions.

### REFERENCE TO SEQUENCE LISTING SUBMITTED ELECTRONICALLY

This application contains a sequence listing, which is submitted electronically via EFS-Web as an ASCII formatted sequence listing with a file name “4US3 Sequence Listing” and a creation date of Mar. 22, 2022, and having a size of 187 kb. The sequence listing submitted via EFS-Web is part of the specification and is herein incorporated by reference in its entirety.

### BACKGROUND

T cell development in the thymus progresses through an ordered process of cell fate decisions. As precursor cells enter the thymus (having arrived from the bone marrow via the portal vasculature), they encounter a series of membrane-associated and soluble proteins that provide critical inputs. These proteins provide signals to the developing thymocytes (T cell precursors). Some important signaling families include Notch, Flt3, Wnt, and cytokines. Notch signaling, in particular, plays a key role in driving precursor cells towards a T cell fate. In the human thymus, the Notch family proteins DLL1, DLL4, and Jag2 (expressed by stromal cells in the thymus) signal through the receptor Notch1 (expressed by early thymocytes). DLL1, DLL4 and Jag2 are thus described as Notch ligands.

While the precise roles of individual Notch ligands or combinations of Notch ligands in the human thymus remain ill-defined, it is evident from in vitro studies that Notch ligands are essential for commitment to the T cell lineage. Once a precursor cell commits to becoming a T cell, the first major bifurcation into two different types of T cells occurs. The human genome possesses four unique T cell receptor (TCR) gene clusters; alpha (α), beta (β), gamma (γ), and delta (δ). In their germline structure, none of these gene clusters can be expressed as a functional transcript that encodes a functional protein. Instead, each of these gene clusters must undergo a physical rearrangement whereby distant sub-genes are fused together through a process called TCR rearrangement. When a TCR gene cluster is “re-arranged” it has the potential to produce a functional protein. Since each functional TCR protein is a heterodimeric pair, at least two TCR gene clusters must be rearranged. There are two types of TCR heterodimers—αβ where an alpha cluster and beta cluster both rearrange to create a functional heterodimer, and γδ where a gamma cluster and delta cluster both rearrange to create a functional heterodimer. How a developing T cell decides to rearrange one cluster rather than another remains unknown, however it is known that thymocytes select one fate or the other—they become αβ or γδ T cells.

Commitment to the γδ T cell lineage occurs early in thymic development. Unlike αβ T cells, γδ T cells are not dependent on the highly-variable classical human leukocyte antigen (HLA) molecules for development or function. So called innate-like T cells, γδ T cells respond to invariant ligands when sensing danger (infection or cancer). As such, γδ T cells are unlikely to cause graft versus host disease when infused into an HLA-mismatched recipient. Thus, γδ T cells provide a significant advantage for allogeneic cell therapies.

Described herein are methods to generate γδ T cells from induced pluripotent stem cells (iPSCs). Furthermore, methods and processes to express a chimeric antigen receptor (CAR) that endows iPSC-derived γδ T cells (γδ iT cells) with the ability to recognize and kill malignant cancer cells are described.

### BRIEF SUMMARY

In one general aspect, provided is a genetically engineered induced pluripotent stem cell or a derivative cell thereof. The cell comprises: (i) one or more polynucleotides encoding a recombinant rearranged γδ T cell receptor (TCR); and (ii) a polynucleotide encoding a chimeric antigen receptor (CAR);

wherein the recombinant rearranged γδ TCR is not specific to the binding target of the CAR and supports differentiation of the iPSC to a T cell.

In certain embodiments, the recombinant rearranged γδ TCR enables expansion of the differentiated T cell after mitogenic stimulation.

In certain embodiments, the one or more polynucleotides encoding the recombinant rearranged γδ TCR comprise a γ TCR variable gene selected from a group consisting of TRGV2-5, TRGV8 and TRGV9 genes; a γ TCR joining gene selected from the group consisting of TRGJ1, TRGJ2, TRGJP, TRGJP1 and TRGJP2 genes; and a γ TCR constant genes selected from the group consisting of TRGC1 and TRGC2 genes. In certain embodiments, the one or more polynucleotides encoding the recombinant rearranged γδ TCR comprise a δ TCR variable gene selected from the group consisting of TRDV1-3 genes; δ TCR diversity genes selected from the group consisting of TRDD1, TRDD2 and TRDD3 genes; δ TCR joining genes selected from the group consisting of TRDJ1, TRDJ2, TRDJ3 and TRDJ4; and a δ TCR constant gene TRDC.

In certain embodiments, the one or more polynucleotides encoding the recombinant rearranged γδ TCR comprise a γ TCR variable gene TRGV9 and a δ TCR variable gene TRDV2.

In certain embodiments, the recombinant rearranged γδ TCR is activated by one or more phospho-antigens selected from isopentenyl pyrophosphate (IPP), dimethylallyl diphosphate (DMAPP), and (E)-4-hydroxy-3-methyl-but-2-enylpyrophosphate (HMBPP), or chemically similar molecules, wherein the phospho-antigens are naturally-occurring in cells as products of metabolic processes or the phospho-antigens are caused to accumulate in cells at higher levels due to treatment with bisphosphonate chemicals, wherein the activity of the phospho-antigens is through direct interaction with the γδ TCR or the activity of phospho-antigens is through interactions with butyrophilin (BTN) proteins BTN2A1, BTN3A1, BTN3A2, or BTN3A3.

In certain embodiments, the recombinant rearranged γδ TCR is not activated by phospho-antigens.

In certain embodiments, the iPSC is reprogrammed from peripheral blood mononuclear cells (PBMCs), preferably CD34+ hematopoietic stem cells (HSCs), αβ T cells or γδ T cells.

In certain embodiments, the iPSC is prepared by expanding the PBMCs in the presence of an amino-bisphosphonate and interleukin 2 (IL2) prior to incorporating reprogramming transcription factors into the PBMC to generate the iPSC.

In certain embodiments, the amino-bisphosphonate is zoledronic acid or salts thereof.

Also provided is a T cell derived from the iPSC of the application.

Also provided is an induced pluripotent stem cell (iPSC) or a T cell derived therefrom comprising one or more polynucleotides encoding a rearranged γδ T cell receptor (TCR) and an exogenous polynucleotide encoding a chimeric antigen receptor (CAR); and one or more of the following additional features:
(a) an exogenous polynucleotide encoding an artificial cell death polypeptide;
(b) a deletion or reduced expression of one or more of B2M, TAP 1, TAP 2, Tapasin, RFXANK, CIITA, RFX5 and RFXAP genes;
(c) a deletion or reduced expression of RAG1 and RAG2 genes;
(d) an exogenous polynucleotide encoding a non-naturally occurring variant of FcγRIII (CD16);
(e) an exogenous polynucleotide encoding interleukin 15 (IL-15) and/or interleukin (IL-15) receptor or a variant or truncation thereof;
(f) an exogeneous polynucleotide encoding a constitutively active IL-7 receptor or variant thereof;
(g) an exogenous polynucleotide encoding interleukin 12 (IL-12) or interleukin 21 (IL-21) or a variant thereof;
(h) an exogenous polynucleotide encoding human leukocyte antigen E (HLA-E) and/or human leukocyte antigen G (HLA-G);
(i) an exogenous polynucleotide encoding leukocyte surface antigen cluster of differentiation CD47 (CD47) and/or CD24; or
(j) an exogenous polynucleotide encoding one or more imaging or reporter proteins, such as PSMA or HSV-tk.

In certain embodiments, the iPSC is reprogrammed from a γδ T cell and the rearranged γδ TCR is endogenous to the γδ T cell. In certain embodiments, the rearranged γδ TCR is recombinant.

In certain embodiments, the rearranged γδ TCR enables increased expansion of the differentiated T cell after mitogenic stimulation than the T cell without the rearranged TCR.

In certain embodiments, the iPSC or T cell further comprises an exogenous polynucleotide encoding a human leukocyte antigen E (HLA-E) and/or human leukocyte antigen G (HLA-G). In certain embodiments, the HLA-E comprises an amino acid sequence having at least 90%, 91%, 92%, 93%, 94%, 95%, 96%, 97%, 98%, 99% or 100% sequence identity to SEQ ID NO: 66 or the HLA-G comprises an amino acid sequence having at least 90%, 91%, 92%, 93%, 94%, 95%, 96%, 97%, 98%, 99% or 100% sequence identity to SEQ ID NO: 69.

In certain embodiments, one or more of the exogenous polynucleotides are integrated at one or more loci on the chromosome of the cell selected from the group consisting of AAVS1, CCR5, ROSA26, collagen, HTRP, Hl 1, GAPDH, RUNX1, B2M, TAPI, TAP2, Tapasin, NLRC5, CIITA, RFXANK, CIITA, RFX5, RFXAP, TRAC, TRBC1, TRBC2, RAG1, RAG2, NKG2A, NKG2D, CD38, CIS, CBL-B, SOCS2, PD1, CTLA4, LAG3, TIM3, or TIGIT genes, provided at least one of the exogenous polynucleotides is integrated at a locus of a gene selected from the group consisting of B2M, TAP 1, TAP 2, Tapasin, RFXANK, CIITA, RFX5 and RFXAP genes to thereby result in a deletion or reduced expression of the gene. In some embodiments, one or more of the exogenous polynucleotides are integrated at a CD38 locus, thereby resulting in a deletion or reduced expression of the CD38 gene.

In certain embodiments, one or more of the exogenous polynucleotides are integrated at the loci of the CIITA, AAVS1 and B2M genes. In certain embodiments, the iPSC or T cell has a deletion or reduced expression of one or more of B2M or CIITA genes.

In certain embodiments, the CAR comprises:

(i) a signal peptide comprising a signal peptide; (ii) an extracellular domain comprising a binding domain that specifically binds an antigen on a target cell; (iii) a hinge region; (iv) a transmembrane domain; (v) an intracellular signaling domain; and (vi) a co-stimulatory domain. In certain embodiments, the signal peptide is GMCSFR signal peptide. In certain embodiments, the extracellular domain comprises an scFv or V_{H}H derived from an antibody that specifically binds an antigen that is expressed on cancer cells, such as the CD19 antigen. In certain embodiments, the hinge region comprises a CD28 hinge region, a CD8 hinge region, or an IgG hinge region. In certain embodiments, the transmembrane domain comprises a CD28 transmembrane domain or a CD8 transmembrane domain. In certain embodiments, the intracellular signaling domain is derived from DAP10, DAP12, Fc epsilon receptor I γ chain (FCER1G), FcR β, NKG2D, CD3δ, CD3ε, CD3γ, CD3ζ, CD5, CD22, CD226, CD66d, CD79A, or CD79B. In certain embodiments, the co-stimulatory domain is derived from CD28, 41BB, IL2Rb, CD40, OX40 (CD134), CD80, CD86, CD27, ICOS, NKG2D, DAP10, DAP12, or 2B4 (CD244).

In certain embodiments, the CAR comprises:
(i) the signal peptide comprising an amino acid sequence having at least 90%, 91%, 92%, 93%, 94%, 95%, 96%, 97%, 98%, 99% or 100% sequence identity to SEQ ID NO: 1;
(ii) the extracellular domain comprising an amino acid sequence having at least 90%, 91%, 92%, 93%, 94%, 95%, 96%, 97%, 98%, 99% or 100% sequence identity to SEQ ID NO: 7;
(iii) the hinge region comprising an amino acid sequence having at least 90%, 91%, 92%, 93%, 94%, 95%, 96%, 97%, 98%, 99% or 100% sequence identity to SEQ ID NO: 22;
(iv) the transmembrane domain comprising an amino acid sequence having at least 90%, 91%, 92%, 93%, 94%, 95%, 96%, 97%, 98%, 99% or 100% sequence identity to SEQ ID NO: 24;
(v) the intracellular signaling domain comprising an amino acid sequence having at least 90%, 91%, 92%, 93%, 94%, 95%, 96%, 97%, 98%, 99% or 100% sequence identity to SEQ ID NO: 6; and
(vi) the co-stimulatory domain comprising an amino acid sequence having at least 90%, 91%, 92%, 93%, 94%, 95%, 96%, 97%, 98%, 99% or 100% sequence identity to SEQ ID NO: 20.

In certain embodiments, the CAR comprises:
(i) the signal peptide comprising the amino acid sequence of SEQ ID NO: 1;
(ii) the extracellular domain comprising the amino acid sequence of SEQ ID NO: 7;
(iii) the hinge region comprising an amino acid sequence of SEQ ID NO: 22;
(iv) the transmembrane domain comprising the amino acid sequence of SEQ ID NO: 24;
(v) the intracellular signaling domain comprising the amino acid sequence of SEQ ID NO: 6; and
(vi) the co-stimulatory domain comprising the amino acid sequence of SEQ ID NO: 20.

In certain embodiments, the mechanism of action of the artificial cell death polypeptide is metabolic, dimerization-inducing or therapeutic monoclonal antibody-mediated. In certain embodiments, the therapeutic monoclonal antibody mediated artificial cell death polypeptide is an inactivated cell surface protein selected from the group of monoclonal antibody specific epitopes selected from epitopes specifically recognized by ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, polatuzumab vedotin, ranibizumab, tocilizumab, trastuzumab, vedolizumab, adalimumab, belimumab, canakinumab, denosumab, golimumab, ipilimumab, ofatumumab, panitumumab, daratumumab, or ustekinumab.

In certain embodiments, the inactivated cell surface protein is a truncated epithelial growth factor (tEGFR) variant. In certain embodiments, the tEGFR variant consists of an amino acid sequence having at least 90%, 91%, 92%, 93%, 94%, 95%, 96%, 97%, 98%, 99% or 100% sequence identity to SEQ ID NO: 71.

In certain embodiments, the rearranged γδ TCR comprises:
(a) a γ TCR chain having a CDR3 of the amino acid sequence of SEQ ID NO: 109, and a δ TCR chain having a CDR3 of the amino acid sequence of SEQ ID NO: 110;
(b) a γ TCR chain having a CDR3 of the amino acid sequence of SEQ ID NO: 111, and a δ TCR chain having a CDR3 of the amino acid sequence of SEQ ID NO: 112;
(c) a γ TCR chain having a CDR3 of the amino acid sequence of SEQ ID NO: 113, and a δ TCR chain having a CDR3 of the amino acid sequence of SEQ ID NO: 114;
(d) a γ TCR chain having a CDR3 of the amino acid sequence of SEQ ID NO: 115, and a δ TCR chain having a CDR3 of the amino acid sequence of SEQ ID NO: 116;
(e) a γ TCR chain having a CDR3 of the amino acid sequence of SEQ ID NO: 117, and a δ TCR chain having a CDR3 of the amino acid sequence of SEQ ID NO: 118;
(f) a γ TCR chain having a CDR3 of the amino acid sequence of SEQ ID NO: 119, and a δ TCR chain having a CDR3 of the amino acid sequence of SEQ ID NO: 120;
(g) a γ TCR chain having a CDR3 of the amino acid sequence of SEQ ID NO: 121, and a δ TCR chain having a CDR3 of the amino acid sequence of SEQ ID NO: 122;
(h) a γ TCR chain having a CDR3 of the amino acid sequence of SEQ ID NO: 123, and a δ TCR chain having a CDR3 of the amino acid sequence of SEQ ID NO: 124;
(i) a γ TCR chain having a CDR3 of the amino acid sequence of SEQ ID NO: 125, and a δ TCR chain having a CDR3 of the amino acid sequence of SEQ ID NO: 126;
(j) a γ TCR chain having a CDR3 of the amino acid sequence of SEQ ID NO: 127, and a δ TCR chain having a CDR3 of the amino acid sequence of SEQ ID NO: 128;
(k) a γ TCR chain having a CDR3 of the amino acid sequence of SEQ ID NO: 129, and a δ TCR chain having a CDR3 of the amino acid sequence of SEQ ID NO: 130;
(l) a γ TCR chain having a CDR3 of the amino acid sequence of SEQ ID NO: 131, and a δ TCR chain having a CDR3 of the amino acid sequence of SEQ ID NO: 132;
(m) a γ TCR chain having a CDR3 of the amino acid sequence of SEQ ID NO: 152, and a δ TCR chain having a CDR3 of the amino acid sequence of SEQ ID NO: 153; or
(n) a γ TCR chain having a CDR3 of the amino acid sequence of SEQ ID NO: 154, and a δ TCR chain having a CDR3 of the amino acid sequence of SEQ ID NO: 155.

In certain embodiments, the rearranged γδ TCR comprises:
(a) the γ TCR chain comprising the amino acid sequence encoded by TRGV9 and TRGJP genes, and having the CDR3 of the amino acid sequence of SEQ ID NO: 109, and the δ TCR chain comprising the amino acid sequence encoded by TRDV2, TRDD3, and TRDJ1 genes, and having the CDR3 of the amino acid sequence of SEQ ID NO: 110;
(b) the γ TCR chain comprising the amino acid sequence encoded by TRGV9, and TRGJP genes, and having the CDR3 of the amino acid sequence of SEQ ID NO: 111, and the δ TCR chain comprising the amino acid sequence encoded by TRDV2, and TRDJ1 genes, and having the CDR3 of the amino acid sequence of SEQ ID NO: 112;
(c) the γ TCR chain comprising the amino acid sequence encoded by TRGV9, and TRGJP genes, and having the CDR3 of the amino acid sequence of SEQ ID NO: 113, and the δ TCR chain comprising the amino acid sequence encoded by TRDV2, TRDD3, and TRDJ1 genes, and having the CDR3 of the amino acid sequence of SEQ ID NO: 114;
(d) the γ TCR chain comprising the amino acid sequence encoded by TRGV9 and TRGJP genes, and having the CDR3 of the amino acid sequence of SEQ ID NO: 115, and the δ TCR chain comprising the amino acid sequence encoded by TRDV2, TRDD3 and TRDJ1 genes, and having the CDR3 of the amino acid sequence of SEQ ID NO: 116;
(e) the γ TCR chain comprising the amino acid sequence encoded by TRGV9 and TRGJP genes, and having the CDR3 of the amino acid sequence of SEQ ID NO: 117, and the δ TCR chain comprising the amino acid sequence encoded by TRDV2, TRDD3 and TRDJ1 genes, and having the CDR3 of the amino acid sequence of SEQ ID NO: 118;
(f) the γ TCR chain comprising the amino acid sequence encoded by TRGV9 and TRGJP genes, and having the CDR3 of the amino acid sequence of SEQ ID NO: 119, and the δ TCR chain comprising the amino acid sequence encoded by TRDV2, TRDD3 and TRDJ1 genes, and having the CDR3 of the amino acid sequence of SEQ ID NO: 120;
(g) the γ TCR chain comprising the amino acid sequence encoded by TRGV9 and TRGJP genes, and having the CDR3 of the amino acid sequence of SEQ ID NO: 121, and the δ TCR chain comprising the amino acid sequence encoded by TRDV2, TRDD3 and TRDJ1 genes, and having the CDR3 of the amino acid sequence of SEQ ID NO: 122;
(h) the γ TCR chain comprising the amino acid sequence encoded by TRGV9 and TRGJP, and having the CDR3 of the amino acid sequence of SEQ ID NO: 123, and the δ TCR chain comprising the amino acid sequence encoded by TRDV2, TRDD3 and TRDJ1 genes, and having the CDR3 of the amino acid sequence of SEQ ID NO: 124;
(i) the γ TCR chain comprising the amino acid sequence encoded by TRGV9 and TRGJP genes, and having the CDR3 of the amino acid sequence of SEQ ID NO: 125, and the δ TCR chain comprising the amino acid sequence encoded by TRDV2, TRDD3 and TRDJ1 genes, and having the CDR3 of the amino acid sequence of SEQ ID NO: 126;
(j) the γ TCR chain comprising the amino acid sequence encoded by TRGV9 and TRGJP genes, and having the CDR3 of the amino acid sequence of SEQ ID NO: 127, and the δ TCR chain comprising the amino acid sequence encoded by TRDV2, TRDD3 and TRDJ1 genes, and having the CDR3 of the amino acid sequence of SEQ ID NO: 128;
(k) the γ TCR chain comprising the amino acid sequence encoded by TRGV9 and TRGJP genes, and having the CDR3 of the amino acid sequence of SEQ ID NO: 129, and the δ TCR chain comprising the amino acid sequence encoded by TRDV2, TRDD3 and TRDJ1 genes, and having the CDR3 of the amino acid sequence of SEQ ID NO: 130;
(l) the γ TCR chain comprising the amino acid sequence encoded by TRGV9 and TRGJP genes, and having the CDR3 of the amino acid sequence of SEQ ID NO: 131, and the δ TCR chain comprising the amino acid sequence encoded by TRDV2, TRDD3 and TRDJ1 genes, and having the CDR3 of the amino acid sequence of SEQ ID NO: 132;
(m) the γ TCR chain comprising the amino acid sequence encoded by TRGV9 and TRGJP genes, and having the CDR3 of the amino acid sequence of SEQ ID NO: 152, and the δ TCR chain comprising the amino acid sequence encoded by TRDV2, TRDD3 and TRDJ1 genes, and having the CDR3 of the amino acid sequence of SEQ ID NO: 153; or
(n) the γ TCR chain comprising the amino acid sequence encoded by TRGV9 and TRGJP genes, and having the CDR3 of the amino acid sequence of SEQ ID NO: 154, and the δ TCR chain comprising the amino acid sequence encoded by TRDV2, TRDD3 and TRDJ1 genes, and having the CDR3 of the amino acid sequence of SEQ ID NO: 155.

In certain embodiments, the recombinant rearranged γδ TCR comprises
(a) a γ TCR chain comprising an amino acid sequence having at least 90%, 91%, 92%, 93%, 94%, 95%, 96%, 97%, 98%, 99% or 100% sequence identity to SEQ ID NO: 133, and a δ TCR chain comprising an amino acid sequence having at least 90%, 91%, 92%, 93%, 94%, 95%, 96%, 97%, 98%, 99% or 100% sequence identity to SEQ ID NO: 134;
(b) a γ TCR chain comprising an amino acid sequence having at least 90%, 91%, 92%, 93%, 94%, 95%, 96%, 97%, 98%, 99% or 100% sequence identity to SEQ ID NO: 135, and a δ TCR chain comprising an amino acid sequence having at least 90%, 91%, 92%, 93%, 94%, 95%, 96%, 97%, 98%, 99% or 100% sequence identity to SEQ ID NO: 136; or
(c) a γ TCR chain comprising an amino acid sequence having at least 90%, 91%, 92%, 93%, 94%, 95%, 96%, 97%, 98%, 99% or 100% sequence identity to SEQ ID NO: 150, and a δ TCR chain comprising an amino acid sequence having at least 90%, 91%, 92%, 93%, 94%, 95%, 96%, 97%, 98%, 99% or 100% sequence identity to SEQ ID NO: 151.

Also provided is an induced pluripotent stem cell (iPSC) or T cell comprising: (i) an exogenous polynucleotide encoding a chimeric antigen receptor (CAR) having the amino acid sequence of SEQ ID NO: 61; (ii) an exogenous polynucleotide encoding an artificial cell death polypeptide comprising an apoptosis-inducing domain having the amino acid sequence of SEQ ID NO: 71; (iii) one or more polynucleotides encoding a rearranged T cell receptor (TCR) locus comprising a γ TCR having the amino acid sequence of SEQ ID NO: 133, 135, or 150, and a δ TCR having the amino acid sequence of SEQ ID NO: 134, 136, or 151; and (iv) optionally, an exogenous polynucleotide encoding a human leukocyte antigen E (HLA-E) having the amino acid sequence of SEQ ID NO: 66; wherein one or more of the exogenous polynucleotides are integrated at loci of AAVS1, CIITA and B2M genes, to thereby delete or reduce expression of CIITA and B2M.

Also provided is a composition comprising the cells according to embodiments of the application. In certain embodiments, the composition further comprises or is used in combination with, one or more therapeutic agents selected from the group consisting of a peptide, a cytokine, a checkpoint inhibitor, a mitogen, a growth factor, a small RNA, a dsRNA (double stranded RNA), siRNA, oligonucleotide, mononuclear blood cells, a vector comprising one or more polynucleic acids of interest, an antibody, a chemotherapeutic agent or a radioactive moiety, or an immunomodulatory drug (IMiD).

Also provided is a method of treating cancer in a subject in need thereof, the method comprising administering a cell of the application or a composition of the application to the subject in need thereof. In certain embodiments, the cancer is non-Hodgkin's lymphoma (NHL).

Also provided is a method of manufacturing a T cell of the application comprising differentiating an iPSC cell of the application under conditions for cell differentiation to thereby obtain the T cell. In certain embodiments, the iPSC is obtained by genomic engineering an iPSC, wherein the genomic engineering comprises targeted editing. Examples of targeted editing include, but are not limited to, deletion, insertion, or in/del carried out by CRISPR, ZFN, TALEN, homing nuclease, homology recombination, or any other functional variation of these methods.

Also provided is a CD34+ hematopoietic progenitor cell (HPC) derived from an induced pluripotent stem cell (iPSC) comprising one or more polynucleotides encoding a rearranged γδ T cell receptor (TCR) and an exogenous polynucleotide encoding a chimeric antigen receptor (CAR); and one or more of the following additional features:
(a) an exogenous polynucleotide encoding an artificial cell death polypeptide;
(b) a deletion or reduced expression of one or more of B2M, TAP 1, TAP 2, Tapasin, RFXANK, CIITA, RFX5 and RFXAP genes;
(c) a deletion or reduced expression of RAG1 and RAG2 genes;
(d) an exogenous polynucleotide encoding a non-naturally occurring variant of FcγRIII (CD16);
(e) an exogenous polynucleotide encoding interleukin 15 (IL-15) and/or interleukin (IL-15) receptor or a variant or truncation thereof;
(f) an exogeneous polynucleotide encoding a constitutively active interleukin 7 (IL-7) receptor or variant thereof;
(g) an exogenous polynucleotide encoding interleukin 12 (IL-12) or interleukin 21 (IL-21) or a variant thereof;
(h) an exogenous polynucleotide encoding human leukocyte antigen E (HLA-E) and/or human leukocyte antigen G (HLA-G);
(i) an exogenous polynucleotide encoding leukocyte surface antigen cluster of differentiation CD47 (CD47) and/or CD24; or
(j) an exogenous polynucleotide encoding one or more imaging or reporter proteins, such as PSMA or HSV-tk.

### BRIEF DESCRIPTION OF THE DRAWINGS

The foregoing summary, as well as the following detailed description of preferred embodiments of the present application, will be better understood when read in conjunction with the appended drawings. It should be understood, however, that the

FIGS. 1A-C show schematics of methods of generating induced pluripotent stem cells (iPSCs) as a source of γδ iT cells. FIG. 1A shows a method of generating γδ T cells using iPSCs derived from mature γδ T cells collected from a blood sample. FIG. 1B shows a method of generating γδ iT cells using iPSCs derived from CD34+ hematopoietic stem cells (HSC) collected from a blood sample. FIG. 1C shows a method of generating γδ iT cells using iPSCs derived from mature Tαβ cells collected from a blood sample.

FIGS. 2A-D show expansion of γδ T cells to derive T cell-derived iPSC (TiPSC). FIG. 2A shows graphs of representative FACS results demonstrating sorting for Vγ9/Vδ2 T cells from CD3+ T cells pre-expansion and after 14 days of expansion in media containing zoledronate and IL-2 (post-expansion). FIG. 2B shows a graph of total cell number over 14 days of expansion in media containing zoledronate and IL-2 (post-expansion). FIG. 2C shows a table depicting the presence or absence of a rearranged Vγ9, Vδ2, or Vδ1 TCR gene locus from four T cell-derived iPSC (TiPSC) lines analyzed using PCR. FIG. 2D shows iT cells that have been differentiated from TiPSC lines (14 days of differentiation from HPC stage). Shown are two Vγ9/Vδ2 TiPSC clones gdTiPSC4.1 and gdTiPSC4.4. Left panels show expression of CD3 and γδ TCR and demonstrate that the majority of cells are CD3/TCR positive at Day 14. Right panels are gated on CD3-positive cells and show that the Vγ9/Vδ2 TCR was uniformly utilized by all developing iT cells.

FIGS. 3A-D show that γδ T cell-derived iPSC (TiPSC) give rise to γδ iT cells when differentiated in vitro. FIGS. 3A-B show graphs of representative FACS results demonstrating that after 14 days of iT differentiation, cells were uniformly (FIG. 3A) CD3-positive and (FIG. 3B) γδ TCR-positive/αβ TCR-negative. FIG. 3C shows a graph of representative FACS results demonstrating CAR expression when the TiPSC line had been engineered to express a CD19-specific CAR under an exogenous constitutive promoter. FIG. 3D shows a graph demonstrating the engineered CAR-iT cells potently killed CD19(+) Reh cells, but did not kill Reh cells where CD19 had been deleted by gene editing (CD19(−)).

FIGS. 4A-B show a comparison of workflows for generating (FIG. 4A) donor-derived and (FIG. 4B) iPSC-derived cell therapies.

FIGS. 5A-B show (FIG. 5A) that exposure of T cells to zoledronic acid and IL-2 results in γδ T cell enrichment (top), however the total increase in γδ T cells is insufficient for successful reprogramming in 2 of 3 donors (bottom), and (FIG. 5B) that a new expansion method retains γδ T cell enrichment (top), while increasing the magnitude of γδ T cell proliferation to levels that are sufficient for iPSC reprogramming in the same 3 donors (bottom).

FIG. 6 shows how the creation of new γδ TiPSC lines begins with collection of donor cells and enrichment of γδ T cells bearing the Vγ9/Vδ2 TCR. Over the course of two weeks of expansion, the frequency of Vγ9/Vδ2 cells is initially diminished (day 7) but rebounds as the cells expand over the course of two weeks. Expanding cells are collected and subjected to iPSC reprogramming through introduction of pluripotency genes. iPSC colonies are identified by microscopic morphology and each colony is picked for subculture. Three experiments with two donors resulted in consistent TiPSC line yield (Table).

FIG. 7A shows that to enable target cell identification, γδ TiPSC lines are engineered with a chimeric antigen receptor (CAR) using CRISPR-mediated gene editing with homology-directed repair (HDR). Then, CAR-TiPSC lines are subjected to the first differentiation process to enforce development of CD34⁺ hematopoietic progenitor cells (HPCs). At this point, the cells are uniformly CD34⁺CD43⁺ and CD45^{+/-}. HPCs are then subjected to the second differentiation process to enforce T lineage commitment and development of γδ CAR-iT cells.

FIG. 7B shows results of flow cytometry analysis on CD34+ hematopoietic progenitor cells (HPCs) to identify CD34+/CD43+ cells (left), CD45+/− cells (middle), and CD19 CAR+(right).

FIG. 7C shows the percentage of CD7+, CD45+, and CD3/TCRγδ+γδ CAR-iT cells following the second differentiation process to enforce T lineage commitment and development γδ CAR-iT cells.

FIG. 7D shows that after four weeks of differentiation, cells express CD45, CD3, γδ TCR, and CD7. Comparison of two different TiPSC lines with different γδ TCRs demonstrates that there is some heterogeneity in the phenotype of CAR-iT that can be obtained through the differentiation process.

FIGS. 8A-D shows in vitro activity of CAR-γδ iT cells. The ability of γδ CAR-iT cells to eliminate CD19-expressing tumors was determined with an IncuCyte assay (tumor killing=decreased signal on y-axis). (FIG. 8A) Activity of γδ CAR-iT was compared to three different donor batches of conventional αβ CAR-T cells using the same CD19 CAR at a 1:1 effector to target ratio using NALM-6 as targets. (FIG. 8B) Activity of the effectors was assessed in the same assay at different E:T ratios. γδ CAR-iT cells performed as well as CAR-T. (FIG. 8C) Activity of γδ CAR-iT was compared to three different donor batches of conventional αβ CAR-T cells using the same CD19 CAR at a 1:1 effector to target ratio using Reh cells as targets. (FIG. 8D) Activity of the effectors was assessed in the same assay at different E:T ratios. γδ CAR-iT cells performed as well as CAR-T.

FIGS. 8E-H shows in vitro activity of CAR-γδ iT cells. Inflammatory cytokine expression (e.g. IFN-γ or TNF) is a concern with cell therapies due to toxicities in patients. Unlike conventional CAR-T cells, γδ CAR-iT cells did not release (FIG. 8E) IFN-γ or (FIG. 8G) TNF when interacting with CD19-expressing tumors cells in a killing assay. (FIG. 8F & FIG. 8H) A CD19-knockout NALM-6 line was used as a control.

FIGS. 8I-J show the importance of homeostatic cytokines for γδ CAR-iT cell persistence and activity, which was evaluated in order to properly design subsequent in vivo studies. Initially, γδ CAR-iT cells were exposed to 1 nM IL-2 or 1 nM IL-5 and cultured for 1 hour with sampling at 0, 10, 30 and 60 minutes. Responsiveness to IL-2 or IL-15 was determined by staining for (FIG. 8I) phosphorylated STAT3 (pSTAT3) and (FIG. 8J) pSTAT5. pSTAT5 mean fluorescence intensity (MFI) was similar for IL-2 and IL-15, but pSTAT3 appeared sooner and to a higher magnitude with IL-15 compared to IL-2.

FIGS. 8K-L show results from a serial killing assay used to evaluate the ability of γδ CAR-iT cells to kill over time and repeated target exposure, which was performed on an IncuCyte where targets but not effectors were replenished daily for 14 days. γδ CAR-iT cells were able to control tumor for 14 rounds with (FIG. 8K) IL-2 or (FIG. 8L) 11-15. However, killing appeared to be more rapid and complete in the presence of IL-15.

FIG. 8M shows results at the end of serial killing, when effector cells were quantified and it was apparent that IL-15 but not IL-2 supported γδ CAR-iT expansion during killing.

FIG. 9A shows a diagram of a timeline for a study designed to evaluate the efficacy of γδ CAR-iT cells in vivo using single versus multiple dosing of effector cells (schema) in NOD.Cg-Prkdc^{scid} Il2rg^{tm1Sug} Tg(CMV-IL2/IL15)1-1Jic/JicTac mice. The results of this study are provided in FIGS. 9B-E. The primary outcome (tumor growth inhibition at day 21) was achieved with significant reduction of luciferase-labeled NALM-6 tumor in the animals when one or three doses of γδ CAR-iT cells were administered (86% TGI and 95% TGI, respectively). Multiple dosing resulted in improved survival of the mice and enhanced persistence of γδ CAR-iT cells. Overall, γδ CAR-iT cells exhibited activity and persistence without evidence of toxicity in the form of body weight loss.

FIG. 9B shows results of a study designed to evaluate the efficacy of γδ CAR-iT cells in vivo using single versus multiple dosing of effector cells (schema) in NOD.Cg-Prkdc^{scid} Il2rg^{tm1Sug} Tg(CMV-IL2/IL15)1-1Jic/JicTac mice. FIG. 9B shows luciferase-labeled NALM-6 tumor growth in the animals when zero (CTL), one (1× dose) or three (3× dose) doses of γδ CAR-iT cells were administered (86% TGI and 95% TGI, respectively).

FIG. 9C shows results of a study designed to evaluate the efficacy of γδ CAR-iT cells in vivo using single versus multiple dosing of effector cells (schema) in NOD.Cg-Prkdc^{scid} Il2rg^{tm1Sug} Tg(CMV-IL2/IL15)1-1Jic/JicTac mice. FIG. 9C shows tumor burden over time in the animals when zero (CTL), one (1× dose) or three (3× dose) doses of γδ CAR-iT cells were administered (86% TGI and 95% TGI, respectively). Overall, γδ CAR-iT cells exhibited activity without evidence of toxicity in the form of body weight loss.

FIG. 9D shows results of a study designed to evaluate the efficacy of γδ CAR-iT cells in vivo using single versus multiple dosing of effector cells (schema) in NOD.Cg-Prkdc^{scid} Il2rg^{tm1Sug} Tg(CMV-IL2/IL15)1-1Jic/JicTac mice. FIG. 9D shows that multiple dosing resulted in improved survival of the mice and enhanced persistence of γδ CAR-iT cells. Overall, γδ CAR-iT cells exhibited activity without evidence of toxicity in the form of body weight loss.

FIG. 9E shows results of a study designed to evaluate the efficacy of γδ CAR-iT cells in vivo using single versus multiple dosing of effector cells (schema) in NOD.Cg-Prkdc^{scid} Il2rg^{tm1Sug} Tg(CMV-IL2/IL15)1-1Jic/JicTac mice. FIG. 9E shows CAR-iT persistence over time in the animals when zero (CTL), one (1× dose) or three (3× dose) doses of γδ CAR-iT cells were administered. Overall, γδ CAR-iT cells exhibited persistence without evidence of toxicity in the form of body weight loss.

FIG. 10 shows a diagram of a dual-color incucyte assay for determining γδ iT cell antibody-dependent cellular cytotoxicity (ADCC) when used in combination with various antibodies (e.g., anti-CD20 antibodies).

FIGS. 11A-B show iPSC-derived CAR-γδ T cells performing ADCC. (FIG. 11A) CAR-γδ T cell performing ADCC of the Raji lymphoblastic B cell line, which normally expresses B cell antigens CD19 and CD20 and was engineered with a transgene encoding a red fluorescent protein. Two different anti-CD20 antibodies (Rituximab and Obinutuzumab) were tested in comparison to their relevant isotype controls. Red tumor cells (CD19+) were eliminated by CAR-γδ iT cells in all conditions. (FIG. 11B) CAR-γδ T cell performing ADCC of Raji cells that were modified using CRISPR gene editing to knockout (KO) the gene encoding CD19, and then engineered with a transgene encoding a green fluorescent protein. Two different anti-CD20 antibodies (Rituximab and Obinutuzumab) were tested in comparison to their relevant isotype controls. Green CD19KO tumor cells were spared from killing in the presence of isotype control antibodies. The presence of Rituximab or Obinutuzumab enabled CAR-γδ iT cells to kill CD19KO cells via ADCC.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this application pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the application described herein. Such equivalents are intended to be encompassed by the application.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., CAR polypeptides and the CAR polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide, protein, or cell) has been substantially separated, produced apart from, or purified away from other biological components of the organism in which the component naturally occurs, i.e., other chromosomal and extrachromosomal DNA and RNA, proteins, cells, and tissues. Nucleic acids, peptides, proteins, and cells that have been “isolated” thus include nucleic acids, peptides, proteins, and cells purified by standard purification methods and purification methods described herein. “Isolated” nucleic acids, peptides, proteins, and cells can be part of a composition and still be isolated if the composition is not part of the native environment of the nucleic acid, peptide, protein, or cell. The term also embraces nucleic acids, peptides and proteins prepared by recombinant expression in a host cell as well as chemically synthesized nucleic acids.

The term “recombinant” refers to a biomolecule that (1) has been removed from its naturally occurring environment, (2) is not associated with all or a portion of another biomolecule in which the biomolecule is found in nature, (3) is operatively linked to another biomolecule which it is not linked to in nature, or (4) does not occur in nature. Examples of biomolecule include, e.g., a nucleic acid or a polypeptide. The term “recombinant” can be used in reference to cloned DNA isolates, chemically synthesized polynucleotide or polypeptide, or analogs thereof, or polynucleotide or polypeptide, or analogs thereof that are biologically synthesized by heterologous systems, as well as proteins and/or mRNAs encoded by such recombinant nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. The term “vector” as used herein comprises the construct to be delivered. A vector can be a linear or a circular molecule. A vector can be integrating or non-integrating. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

By “integration” it is meant that one or more nucleotides of a construct is stably inserted into the cellular genome, i.e., covalently linked to the nucleic acid sequence within the cell's chromosomal DNA. By “targeted integration” it is meant that the nucleotide(s) of a construct is inserted into the cell's chromosomal or mitochondrial DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” can further comprise replacement of the endogenous sequence or a nucleotide that is deleted with the one or more inserted nucleotides.

As used herein, the term “exogenous” is intended to mean that the referenced molecule or the referenced activity is introduced into, or non-native to, the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell in its native form. Similarly, the term when used in reference to expression of an encoding nucleic acid refers to expression of an encoding nucleic acid natively contained within the cell and not exogenously introduced.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e. a polypeptide found in nature) or fragment thereof; a variant polypeptide (i.e. a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

“Operably-linked” refers to the association of nucleic acid sequences on a single nucleic acid fragment so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed CAR can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

As used herein, the term “engineered immune cell” refers to an immune cell, also referred to as an immune effector cell, that has been genetically modified by the addition of exogenous genetic material in the form of DNA or RNA to the total genetic material of the cell.

Induced Pluripotent Stem Cells (iPSCs) and Immune Effector Cells

IPSCs have unlimited self-renewing capacity. Use of iPSCs enables cellular engineering to produce a controlled cell bank of modified cells that can be expanded and differentiated into desired immune effector cells, supplying large amounts of homogeneous allogeneic therapeutic products.

Provided herein are genetically engineered iPSCs and derivative cells thereof. The selected genomic modifications provided herein enhance the therapeutic properties of the derivative cells. The derivative cells are functionally improved and suitable for allogeneic off-the-shelf cell therapies following a combination of selective modalities being introduced to the cells at the level of iPSC through genomic engineering. This approach can help to reduce the side effects mediated by CRS/GVHD and prevent long-term autoimmunity while providing excellent efficacy.

In accordance with the invention, the engineered iPSC's hereof are capable of being differentiated into gamma delta T cell immune effector cells. As used herein, the term “differentiation” is the process by which an unspecialized (“uncommitted”) or less specialized cell acquires the features of a specialized cell. Specialized cells include, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the term “pluripotent” refers to the ability of a cell to form all lineages of the body or soma or the embryo proper. For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the term “induced pluripotent stem cells” or, iPSCs, means that the stem cells are produced from differentiated adult, neonatal or fetal cells that have been induced or changed or reprogrammed into cells capable of differentiating into tissues of all three germ or dermal layers: mesoderm, endoderm, and ectoderm. The iPSCs produced do not refer to cells as they are found in nature.

As used herein, the terms “reprogramming” or “dedifferentiation” refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

The term “hematopoietic stem and progenitor cells,” “hematopoietic stem cells,” “hematopoietic progenitor cells,” or “hematopoietic precursor cells” or “HPCs” refers to cells which are committed to a hematopoietic lineage but are capable of further hematopoietic differentiation. Hematopoietic stem cells include, for example, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). As used herein, “CD34+ hematopoietic progenitor cell” refers to an HPC that expresses CD34 on its surface.

As used herein, the term “immune cell” or “immune effector cell” refers to a cell that is involved in an immune response. Immune response includes, for example, the promotion of an immune effector response. Examples of immune cells include T cells, B cells, natural killer (NK) cells, mast cells, and myeloid-derived phagocytes.

As used herein, the terms “T lymphocyte” and “T cell” are used interchangeably and refer to a type of white blood cell that completes maturation in the thymus and that has various roles in the immune system. A T cell can have the roles including, e.g., the identification of specific foreign antigens in the body and the activation and deactivation of other immune cells. A T cell can be any T cell, such as a cultured T cell, e.g., a primary T cell, or a T cell from a cultured T cell line, e.g., Jurkat, SupTl, etc., or a T cell obtained from a mammal. The T cell can be CD3+ cells. The T cell can be any type of T cell and can be of any developmental stage, including but not limited to, CD4+/CD8+ double positive T cells, CD4+ helper T cells (e.g., Thl and Th2 cells), CD8+ T cells (e.g., cytotoxic T cells), peripheral blood mononuclear cells (PBMCs), peripheral blood leukocytes (PBLs), tumor infiltrating lymphocytes (TILs), memory T cells, naive T cells, regulator T cells, gamma delta T cells (γδ T cells), and the like. Additional types of helper T cells include cells such as Th3 (Treg), Thl7, Th9, or Tfh cells. Additional types of memory T cells include cells such as central memory T cells (Tcm cells), effector memory T cells (Tern cells and TEMRA cells). The T cell can also refer to a genetically engineered T cell, such as a T cell modified to express a T cell receptor (TCR) and/or a chimeric antigen receptor (CAR). The T cell can also be differentiated from a stem cell or progenitor cell.

“CD4+ T cells” refers to a subset of T cells that express CD4 on their surface and are associated with cell-mediated immune response. They are characterized by the secretion profiles following stimulation, which can include secretion of cytokines such as IFN-gamma, TNF-alpha, IL2, IL4 and IL10. “CD4” are 55-kD glycoproteins originally defined as differentiation antigens on T-lymphocytes, but also found on other cells including monocytes/macrophages. CD4 antigens are members of the immunoglobulin supergene family and are implicated as associative recognition elements in MEW (major histocompatibility complex) class II-restricted immune responses. On T-lymphocytes they define the helper/inducer subset.

“CD8+ T cells” refers to a subset of T cells which express CD8 on their surface, are MEW class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

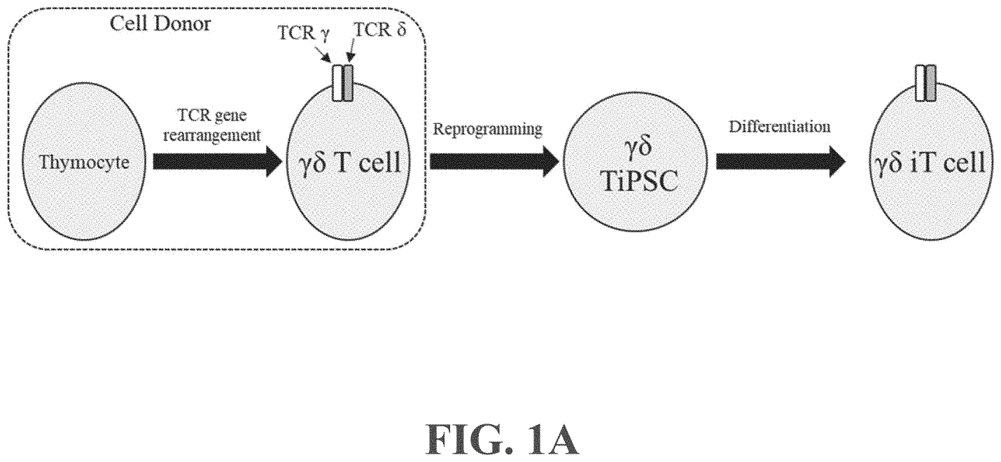

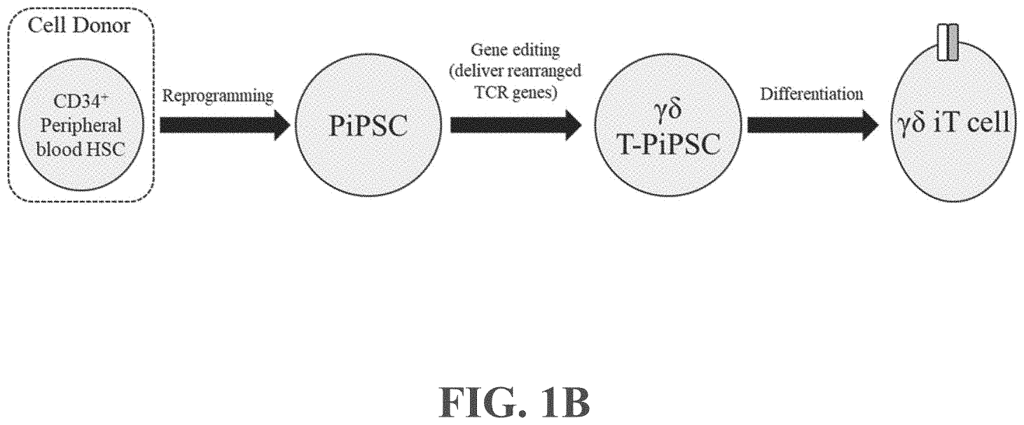

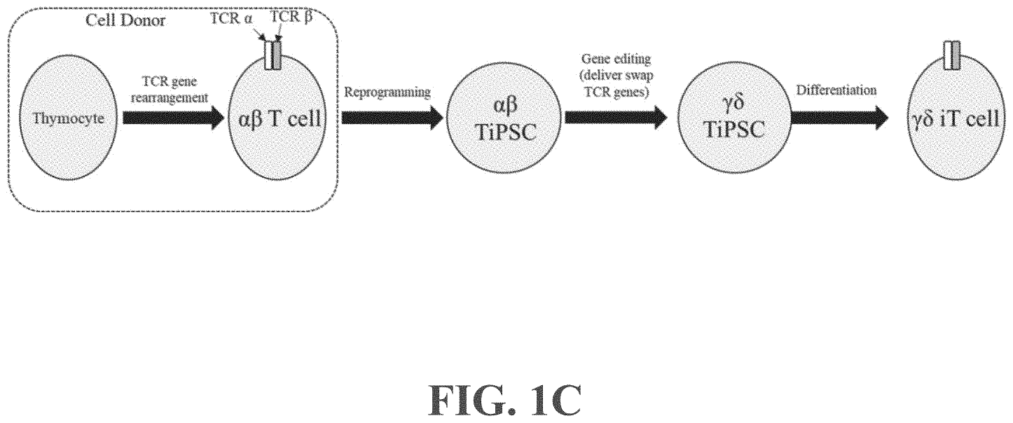

The induced pluripotent stem cell (iPSC) parental cell lines can be generated from peripheral blood mononuclear cells (PBMCs) or T cells using any known method for introducing re-programming factors into non-pluripotent cells using methods known in the art. For instance, the so called “Thompson Factors” as described in U.S. Pat. Nos. 8,183,038, 8,268,620, 8,440,461, 9,499,786, 10,865,381 can be used, or the Yamanaka Factors as described in U.S. Pat. No. 8,952,801 the complete disclosures of which are incorporated herein by reference. Methods include the episomal plasmid-based process as previously described in U.S. Pat. Nos. 8,546,140; 9,644,184; 9,328,332; and 8,765,470, as well as the Sendai virus and other methods as described by Malik, et al Methods Mol Biol. 2013; 997: 23-33, the complete disclosures of which are incorporated herein by reference. The reprogramming factors can be in a form of polynucleotides, and thus are introduced to the non-pluripotent cells by vectors such as a retrovirus, a Sendai virus, an adenovirus, an episome, and a mini-circle. In particular embodiments, the one or more polynucleotides encoding at least one reprogramming factor are introduced by a lentiviral vector. In some embodiments, the one or more polynucleotides introduced by an episomal vector. In various other embodiments, the one or more polynucleotides are introduced by a Sendai viral vector. In some embodiments, the iPSC's are clonal iPSC's or are obtained from a pool of iPSCs and the genome edits are introduced by making one or more targeted integration and/or in/del at one or more selected sites. In another embodiment, the iPSC's are obtained from human T cells having antigen specificity and a reconstituted TCR gene (hereinafter, also referred to as “T-iPS” cells or “T-iPSC”) as described in U.S. Pat. No. 9,206,394, and 10,787,642 hereby incorporated by reference into the present application. FIG. 1A-C show schematics of exemplary methods for generating iPSCs of the application.

As used herein, the term “genetic imprint” refers to genetic or epigenetic information that contributes to preferential therapeutic attributes in a source cell or an iPSC, and is retainable in the source cell derived iPSCs, and/or the iPSC-derived hematopoietic lineage cells. As used herein, “a source cell” is a non-pluripotent cell that can be used for generating iPSCs through reprogramming, and the source cell derived iPSCs can be further differentiated to specific cell types including any hematopoietic lineage cells. The source cell derived iPSCs, and differentiated cells therefrom are sometimes collectively called “derived” or “derivative” cells depending on the context. For example, derivative effector cells or derivative T or “iT” cells, as used throughout this application are cells differentiated from an iPSC, as compared to their primary counterpart obtained from natural/native sources such as peripheral blood, umbilical cord blood, or other donor tissues. As used herein, the genetic imprint(s) conferring a preferential therapeutic attribute is incorporated into the iPSCs either through reprogramming a selected source cell that is donor-, disease-, or treatment response-specific, or through introducing genetically modified modalities to iPSC using genomic editing.

In one general aspect, the application provides an induced pluripotent stem cell (iPSC) that comprises one or more polynucleotides encoding a rearranged γδ TCR, wherein the rearranged γδ TCR supports differentiation of the iPSC to a T cell.

### I. TCR Expression

A T cell receptor (TCR) is a membrane complex found on the surface of T cells that recognizes antigens specifically. It is a heterodimer consisting of alpha (α) and beta (β) chains or gamma (γ) and delta (δ) chains. Each of the alpha, beta, gamma and delta chains of a TCR can be a glycoprotein. As a member of the Ig superfamily, with Ig-like domains, a TCR generates its diversity in a manner similar to that for antibodies, e g., mainly from genetic recombination of the DNA-encoded segments in individual somatic T cells by somatic V(D)J recombination. In a single cell, the T cell receptor loci are rearranged and expressed in the order delta, gamma, beta, and alpha. If both delta and gamma rearrangements produce functional chains, the cell expresses delta and gamma. If not, the cell proceeds to rearrange the beta and alpha loci. However, unlike antibodies, TCR genes do not undergo somatic hypermutation. The TCRα gene locus contains variable (V) and joining (J) gene segments (Vβ and Jβ), whereas the TCRβ locus contains a D gene segment in addition to Vα and Jα segments. Accordingly, the α chain is generated from VJ recombination and the β chain is generated involving VDJ recombination. Similarly, the TCRγ chain is generated involving VJ recombination and the TCRδ gene is generated involving VDJ recombination. The gene segments for TCR are flanked by the same recombination signal sequences as are the Ig gene segments, and the same RAG-1 and RAG-2 encoded recombinase and TdT are required for somatic recombination.

As used herein, a “rearranged TCR” is a TCR encoded by a rearranged TCR gene which has undergone a physical rearrangement whereby distant sub-genes are fused together. The human genome possesses four unique TCR gene clusters; alpha (α), beta (β), gamma (γ), and delta (δ), encoding the TCR alpha, beta, gamma and delta chains, respectively, via rearranged TCR genes. Each chain of the TCR has a variable and a constant region. The variable region contains three hypervariable or complementarity-determining regions (CDRs) and framework residues. CDR3, is mainly responsible for recognizing a processed antigen. To activate T cells, the TCR forms a molecular complex with the CD3 complex, which contains a CD3γ chain, a CD3δ chain, and two CD3εchains.

“Alpha-beta T cell receptors” or “αβ TCR” are antigen specific T cell receptors essential to the immune response and have one α (alpha) chain and one β (beta) chain. Binding of αβ TCR to peptide-major histocompatibility complex (pMHC) initiates TCR-CD3 intracellular activation, recruitment of numerous signaling molecules, and branching and integrating signaling pathways, leading to mobilization of transcription factors that are critical for gene expression and T cell growth and function acquisition. T cells with αß TCRs have specific reactivity to peptides presented via human leukocyte antigen (HLA) system or complex.

“Gamma-delta T cell receptors” or “γδ TCR” are antigen specific T cell receptors present on the cell surface of γδ T cells, having one γ (gamma) chain and one δ (delta) chain. γδ T cells do not necessarily require antigen processing and MHC presentation of peptide epitopes, and they can be activated by non-peptide antigens. γδ T cells can respond to ligands when sensing danger (infection or cancer). γδ T cells display both innate cytotoxic functions and antigen-presenting capability. γδ T cells are activated by γδ TCR ligands (e.g. phospho-antigens) in combination with MHC-associated ligands of the activatory receptor killer cell lectin-like receptor subfamily K, member 1 (KLRK1), also known as NKG2D, such as MHC class I polypeptide-related sequence A (MICA), MICB, and various members of the UL16-binding protein (ULBP) family.

“HLA-restricted antigen recognition,” or “HLA restriction” refers to the fact that a T cell can recognize a foreign peptide bound to a self-major histocompatibility complex molecule, but will only respond to the antigen when it is bound to a particular HLA molecule (e.g., HLA-A*0201). During T cell development, T cells go through a selection process in the thymus to ensure that the TCR will not recognize HLA molecules presenting self-antigens. The selection process results in developed T cells with specific TCRs that only respond to certain HLA molecules but not others (e.g., non-restricted MHC molecules).

As used herein, a “public TCR” or “trusted TCR” is a TCR that comprises a sequence that occurs in multiple individuals with a certain HLA type. These sequences occur so frequently in people who carry the restricting HLA allele, that they have been proven in nature to be compatible with a vast diversity of HLA-I alleles. Thus, these TCRs fail to recognize non-restricted HLA molecules and are unlikely to participate in graft versus host disease. Public TCRs and methods of identifying them have been described by Choo et al., J Virol. 2014 September; 88(18):10613-23; Valkenburg et al., Proc Natl Acad Sci USA. 2016 Apr. 19; 113(16):4440-5; Sant et al., Front Immunol. 2018 Jun. 27; 9:1453; Chen et al., Cell Rep. 2017 Apr. 18; 19(3):569-583; J Biol Chem. 2016 Nov. 18; 291(47):24335-24351; and Song et al., Nat Struct Mol Biol. 2017 April; 24(4):395-406, the relevant disclosures of which are incorporated herein.

The T cell receptor gamma locus (TRG, TCRG, or TRG@) encodes the T cell receptor gamma chain. The human TRG locus is composed of two constant region genes (TRGC), five joining segments (TRGJ) and at least 14 variable γ-genes (TRGV). Several V genes of the gamma locus are known to be incapable of encoding a protein and are considered pseudogenes. During T cell development, a recombination event occurs at the DNA level joining a V gene with a J segment, and the C gene is later joined by splicing at the RNA level. Recombination of different V gene segments with several J segments provides a range of antigen recognition. Additional diversity in antigen recognition is attained by junctional diversity, resulting from the random addition of nucleotides by terminal deoxynucleotidyl transferase. In certain embodiments, a polynucleotide encoding a γ TCR chain comprises a variable gene selected from the group consisting of TRGV2, TRGV3, TRGV4, TRGV5, TRGV8, and TRGV9.

T cell receptor delta locus (TRD, TCRD, TCRDV1 or TRD@) encodes the T cell receptor delta chain. In humans, the δ-locus is embedded within the α-locus on chromosome 14. The human TRD locus is composed of one constant region gene (TRDC), four joining segments (TRDJ) and only three true variable δ-genes (TRDV). In certain embodiments, a polynucleotide encoding a δ TCR chain comprises a variable gene selected from the group consisting of TRDV1, TRDV2, and TRDV3.

In certain embodiments, a polynucleotide encoding the rearranged γ TCR chain comprises or consists of, a polynucleotide sequence having at least 90%, 91%, 92%, 93%, 94%, 95%, 96%, 97%, 98%, 99% or 100% sequence identity to SEQ ID NO: 83 and the polynucleotide encoding the δ TCR chain comprises or consists of a polynucleotide sequence having at least 90%, 91%, 92%, 93%, 94%, 95%, 96%, 97%, 98%, 99% or 100% sequence identity to SEQ ID NO: 84.

In certain embodiments, a polynucleotide encoding the γ TCR chain comprises or consists of a polynucleotide sequence having at least 90%, 91%, 92%, 93%, 94%, 95%, 96%, 97%, 98%, 99% or 100% sequence identity to SEQ ID NO: 85 and the polynucleotide encoding the δ TCR chain comprises or consists of a polynucleotide sequence having at least 90%, 91%, 92%, 93%, 94%, 95%, 96%, 97%, 98%, 99% or 100% sequence identity to SEQ ID NO: 86.

In certain embodiments, a polynucleotide encoding the γ TCR chain comprises or consists of a polynucleotide sequence having at least 90%, 91%, 92%, 93%, 94%, 95%, 96%, 97%, 98%, 99% or 100% sequence identity to SEQ ID NO: 87 and the polynucleotide encoding the δ TCR chain comprises or consists of a polynucleotide sequence having at least 90%, 91%, 92%, 93%, 94%, 95%, 96%, 97%, 98%, 99% or 100% sequence identity to SEQ ID NO: 88.

In certain embodiments, a polynucleotide encoding the γ TCR chain comprises or consists of a polynucleotide sequence having at least 90%, 91%, 92%, 93%, 94%, 95%, 96%, 97%, 98%, 99% or 100% sequence identity to SEQ ID NO: 89 and the polynucleotide encoding the δ TCR chain comprises or consists of a polynucleotide sequence having at least 90%, 91%, 92%, 93%, 94%, 95%, 96%, 97%, 98%, 99% or 100% sequence identity to SEQ ID NO: 90.

In certain embodiments, a polynucleotide encoding the γ TCR chain comprises or consists of a polynucleotide sequence having at least 90%, 91%, 92%, 93%, 94%, 95%, 96%, 97%, 98%, 99% or 100% sequence identity to SEQ ID NO: 91 and the polynucleotide encoding the δ TCR chain comprises or consists of a polynucleotide sequence having at least 90%, 91%, 92%, 93%, 94%, 95%, 96%, 97%, 98%, 99% or 100% sequence identity to SEQ ID NO: 92.

In certain embodiments, a polynucleotide encoding the γ TCR chain comprises or consists of a polynucleotide sequence having at least 90%, 91%, 92%, 93%, 94%, 95%, 96%, 97%, 98%, 99% or 100% sequence identity to SEQ ID NO: 93 and the polynucleotide encoding the δ TCR chain comprises or consists of a polynucleotide sequence having at least 90%, 91%, 92%, 93%, 94%, 95%, 96%, 97%, 98%, 99% or 100% sequence identity to SEQ ID NO: 94.

In certain embodiments, a polynucleotide encoding the γ TCR chain comprises or consists of a polynucleotide sequence having at least 90%, 91%, 92%, 93%, 94%, 95%, 96%, 97%, 98%, 99% or 100% sequence identity to SEQ ID NO: 95 and the polynucleotide encoding the δ TCR chain comprises or consists of a polynucleotide sequence having at least 90%, 91%, 92%, 93%, 94%, 95%, 96%, 97%, 98%, 99% or 100% sequence identity to SEQ ID NO: 96.

In certain embodiments, a polynucleotide encoding the γ TCR chain comprises or consists of a polynucleotide sequence having at least 90%, 91%, 92%, 93%, 94%, 95%, 96%, 97%, 98%, 99% or 100% sequence identity to SEQ ID NO: 97 and the polynucleotide encoding the δ TCR chain comprises or consists of a polynucleotide sequence having at least 90%, 91%, 92%, 93%, 94%, 95%, 96%, 97%, 98%, 99% or 100% sequence identity to SEQ ID NO: 98.

In certain embodiments, a polynucleotide encoding the γ TCR chain comprises or consists of a polynucleotide sequence having at least 90%, 91%, 92%, 93%, 94%, 95%, 96%, 97%, 98%, 99% or 100% sequence identity to SEQ ID NO: 99 and the polynucleotide encoding the δ TCR chain comprises or consists of a polynucleotide sequence having at least 90%, 91%, 92%, 93%, 94%, 95%, 96%, 97%, 98%, 99% or 100% sequence identity to SEQ ID NO: 100.

In certain embodiments, a polynucleotide encoding the γ TCR chain comprises or consists of a polynucleotide sequence having at least 90%, 91%, 92%, 93%, 94%, 95%, 96%, 97%, 98%, 99% or 100% sequence identity to SEQ ID NO: 101 and the polynucleotide encoding the δ TCR chain comprises or consists of a polynucleotide sequence having at least 90%, 91%, 92%, 93%, 94%, 95%, 96%, 97%, 98%, 99% or 100% sequence identity to SEQ ID NO: 102.

In certain embodiments, a polynucleotide encoding the γ TCR chain comprises or consists of a polynucleotide sequence having at least 90%, 91%, 92%, 93%, 94%, 95%, 96%, 97%, 98%, 99% or 100% sequence identity to SEQ ID NO: 103 and the polynucleotide encoding the δ TCR chain comprises or consists of a polynucleotide sequence having at least 90%, 91%, 92%, 93%, 94%, 95%, 96%, 97%, 98%, 99% or 100% sequence identity to SEQ ID NO: 104.

In certain embodiments, a polynucleotide encoding the γ TCR chain comprises or consists of a polynucleotide sequence having at least 90%, 91%, 92%, 93%, 94%, 95%, 96%, 97%, 98%, 99% or 100% sequence identity to SEQ ID NO: 105 and the polynucleotide encoding the δ TCR chain comprises or consists of a polynucleotide sequence having at least 90%, 91%, 92%, 93%, 94%, 95%, 96%, 97%, 98%, 99% or 100% sequence identity to SEQ ID NO: 106.

In certain embodiments, a polynucleotide encoding the γ TCR chain comprises or consists of a polynucleotide sequence having at least 90%, 91%, 92%, 93%, 94%, 95%, 96%, 97%, 98%, 99% or 100% sequence identity to SEQ ID NO: 107 and the polynucleotide encoding the δ TCR chain comprises or consists of a polynucleotide sequence having at least 90%, 91%, 92%, 93%, 94%, 95%, 96%, 97%, 98%, 99% or 100% sequence identity to SEQ ID NO: 108.

In certain embodiments, a polynucleotide encoding the rearranged γ TCR chain comprises or consists of, a polynucleotide sequence having at least 90%, 91%, 92%, 93%, 94%, 95%, 96%, 97%, 98%, 99% or 100% sequence identity to SEQ ID NO: 148 and the polynucleotide encoding the δ TCR chain comprises or consists of a polynucleotide sequence having at least 90%, 91%, 92%, 93%, 94%, 95%, 96%, 97%, 98%, 99% or 100% sequence identity to SEQ ID NO: 149.

In certain embodiments, a rearranged TCR comprises a γ TCR chain having a CDR3 of the amino acid sequence of SEQ ID NO: 109, and a δ TCR chain having a CDR3 of the amino acid sequence of SEQ ID NO: 110.

In certain embodiments, a rearranged TCR comprises a γ TCR chain having a CDR3 of the amino acid sequence of SEQ ID NO: 111, and a δ TCR chain having a CDR3 of the amino acid sequence of SEQ ID NO: 112.

In certain embodiments, a rearranged TCR comprises a γ TCR chain having a CDR3 of the amino acid sequence of SEQ ID NO: 113, and a δ TCR chain having a CDR3 of the amino acid sequence of SEQ ID NO: 114.

In certain embodiments, a rearranged TCR comprises a γ TCR chain having a CDR3 of the amino acid sequence of SEQ ID NO: 115, and a δ TCR chain having a CDR3 of the amino acid sequence of SEQ ID NO: 116.

In certain embodiments, a rearranged TCR comprises a γ TCR chain having a CDR3 of the amino acid sequence of SEQ ID NO: 117, and a δ TCR chain having a CDR3 of the amino acid sequence of SEQ ID NO: 118.

In certain embodiments, a rearranged TCR comprises a γ TCR chain having a CDR3 of the amino acid sequence of SEQ ID NO: 119, and a δ TCR chain having a CDR3 of the amino acid sequence of SEQ ID NO: 120.

In certain embodiments, a rearranged TCR comprises a γ TCR chain having a CDR3 of the amino acid sequence of SEQ ID NO: 121, and a δ TCR chain having a CDR3 of the amino acid sequence of SEQ ID NO: 122.

In certain embodiments, a rearranged TCR comprises a γ TCR chain having a CDR3 of the amino acid sequence of SEQ ID NO: 123, and a δ TCR chain having a CDR3 of the amino acid sequence of SEQ ID NO: 124.

In certain embodiments, a rearranged TCR comprises a γ TCR chain having a CDR3 of the amino acid sequence of SEQ ID NO: 125, and a δ TCR chain having a CDR3 of the amino acid sequence of SEQ ID NO: 126.

In certain embodiments, a rearranged TCR comprises a γ TCR chain having a CDR3 of the amino acid sequence of SEQ ID NO: 127, and a δ TCR chain having a CDR3 of the amino acid sequence of SEQ ID NO: 128.

In certain embodiments, a rearranged TCR comprises a γ TCR chain having a CDR3 of the amino acid sequence of SEQ ID NO: 129, and a δ TCR chain having a CDR3 of the amino acid sequence of SEQ ID NO: 130.

In certain embodiments, a rearranged TCR comprises a γ TCR chain having a CDR3 of the amino acid sequence of SEQ ID NO: 131, and a δ TCR chain having a CDR3 of the amino acid sequence of SEQ ID NO: 132.

In certain embodiments, a rearranged TCR comprises a γ TCR chain having a CDR3 of the amino acid sequence of SEQ ID NO: 152, and a δ TCR chain having a CDR3 of the amino acid sequence of SEQ ID NO: 153.

In certain embodiments, a rearranged TCR comprises a γ TCR chain having a CDR3 of the amino acid sequence of SEQ ID NO: 154, and a δ TCR chain having a CDR3 of the amino acid sequence of SEQ ID NO: 155.

In certain embodiments, a rearranged TCR comprises a γ TCR chain comprising the amino acid sequence encoded by TRGV9 and TRGJP genes, and having a CDR3 of the amino acid sequence of SEQ ID NO: 109, and a δ TCR chain comprising the amino acid sequence encoded by TRDV2, TRDD3, and TRDJ1 genes, and having a CDR3 of the amino acid sequence of SEQ ID NO: 110.

In certain embodiments, a rearranged TCR comprises a γ TCR chain comprising the amino acid sequence encoded by TRGV9, and TRGJP genes, and having a CDR3 of the amino acid sequence of SEQ ID NO: 111, and a δ TCR chain comprising the amino acid sequence encoded by TRDV2, and TRDJ1 genes, and having a CDR3 of the amino acid sequence of SEQ ID NO: 112.

In certain embodiments, a rearranged TCR comprises a γ TCR chain comprising the amino acid sequence encoded by TRGV9, and TRGJP genes, and having a CDR3 of the amino acid sequence of SEQ ID NO: 113, and a δ TCR chain comprising the amino acid sequence encoded by TRDV2, TRDD3, and TRDJ1 genes, and having a CDR3 of the amino acid sequence of SEQ ID NO: 114.

In certain embodiments, a rearranged TCR comprises a γ TCR chain comprising the amino acid sequence encoded by TRGV9 and TRGJP genes, and having a CDR3 of the amino acid sequence of SEQ ID NO: 115, and a δ TCR chain comprising the amino acid sequence encoded by TRDV2, TRDD3 and TRDJ1 genes, and having a CDR3 of the amino acid sequence of SEQ ID NO: 116.

In certain embodiments, a rearranged TCR comprises γ TCR chain comprising the amino acid sequence encoded by TRGV9 and TRGJP genes, and having a CDR3 of the amino acid sequence of SEQ ID NO: 117, and a δ TCR chain comprising the amino acid sequence encoded by TRDV2, TRDD3 and TRDJ1 genes, and having a CDR3 of the amino acid sequence of SEQ ID NO: 118.

In certain embodiments, a rearranged TCR comprises a γ TCR chain comprising the amino acid sequence encoded by TRGV9 and TRGJP genes, and having a CDR3 of the amino acid sequence of SEQ ID NO: 119, and a δ TCR chain comprising the amino acid sequence encoded by TRDV2, TRDD3 and TRDJ1 genes, and having a CDR3 of the amino acid sequence of SEQ ID NO: 120.

In certain embodiments, a rearranged TCR comprises a γ TCR chain comprising the amino acid sequence encoded by TRGV9 and TRGJP genes, and having a CDR3 of the amino acid sequence of SEQ ID NO: 121, and a δ TCR chain comprising the amino acid sequence encoded by TRDV2, TRDD3 and TRDJ1 genes, and having a CDR3 of the amino acid sequence of SEQ ID NO: 122.

In certain embodiments, a rearranged TCR comprises a γ TCR chain comprising the amino acid sequence encoded by TRGV9 and TRGJP, and having a CDR3 of the amino acid sequence of SEQ ID NO: 123, and a δ TCR chain comprising the amino acid sequence encoded by TRDV2, TRDD3 and TRDJ1 genes, and having a CDR3 of the amino acid sequence of SEQ ID NO: 124.

In certain embodiments, a rearranged TCR comprises a γ TCR chain comprising the amino acid sequence encoded by TRGV9 and TRGJP genes, and having a CDR3 of the amino acid sequence of SEQ ID NO: 125, and a δ TCR chain comprising the amino acid sequence encoded by TRDV2, TRDD3 and TRDJ1 genes, and having a CDR3 of the amino acid sequence of SEQ ID NO: 126.

In certain embodiments, a rearranged TCR comprises a γ TCR chain comprising the amino acid sequence encoded by TRGV9 and TRGJP genes, and having a CDR3 of the amino acid sequence of SEQ ID NO: 127, and a δ TCR chain comprising the amino acid sequence encoded by TRDV2, TRDD3 and TRDJ1 genes, and having a CDR3 of the amino acid sequence of SEQ ID NO: 128.

In certain embodiments, a rearranged TCR comprises a γ TCR chain comprising the amino acid sequence encoded by TRGV9 and TRGJP genes, and having a CDR3 of the amino acid sequence of SEQ ID NO: 129, and a δ TCR chain comprising the amino acid sequence encoded by TRDV2, TRDD3 and TRDJ1 genes, and having a CDR3 of the amino acid sequence of SEQ ID NO: 130.

In certain embodiments, a rearranged TCR comprises a γ TCR chain comprising the amino acid sequence encoded by TRGV9 and TRGJP genes, and having a CDR3 of the amino acid sequence of SEQ ID NO: 131, and a δ TCR chain comprising the amino acid sequence encoded by TRDV2, TRDD3 and TRDJ1 genes, and having a CDR3 of the amino acid sequence of SEQ ID NO: 132.

In certain embodiments, a rearranged TCR comprises a γ TCR chain comprising the amino acid sequence encoded by TRGV9 and TRGJP genes, and having a CDR3 of the amino acid sequence of SEQ ID NO: 152, and a δ TCR chain comprising the amino acid sequence encoded by TRDV2, TRDD3, and TRDJ1 genes, and having a CDR3 of the amino acid sequence of SEQ ID NO: 153.

In certain embodiments, a rearranged TCR comprises a γ TCR chain comprising the amino acid sequence encoded by TRGV9 and TRGJP genes, and having a CDR3 of the amino acid sequence of SEQ ID NO: 154, and a δ TCR chain comprising the amino acid sequence encoded by TRDV2, TRDD3, and TRDJ1 genes, and having a CDR3 of the amino acid sequence of SEQ ID NO: 155.

In certain embodiments, a rearranged γδ TCR comprises a γ TCR chain comprising an amino acid sequence having at least 90%, 91%, 92%, 93%, 94%, 95%, 96%, 97%, 98%, 99% or 100% sequence identity to SEQ ID NO: 133, and a δ TCR chain comprising an amino acid sequence having at least 90%, 91%, 92%, 93%, 94%, 95%, 96%, 97%, 98%, 99% or 100% sequence identity to SEQ ID NO: 134.

In certain embodiments, a rearranged γδ TCR comprises a γ TCR chain comprising an amino acid sequence having at least 90%, 91%, 92%, 93%, 94%, 95%, 96%, 97%, 98%, 99% or 100% sequence identity to SEQ ID NO: 135, and a δ TCR chain comprising an amino acid sequence having at least 90%, 91%, 92%, 93%, 94%, 95%, 96%, 97%, 98%, 99% or 100% sequence identity to SEQ ID NO: 136.

In certain embodiments, a rearranged γδ TCR comprises a γ TCR chain comprising an amino acid sequence having at least 90%, 91%, 92%, 93%, 94%, 95%, 96%, 97%, 98%, 99% or 100% sequence identity to SEQ ID NO: 150, and a δ TCR chain comprising an amino acid sequence having at least 90%, 91%, 92%, 93%, 94%, 95%, 96%, 97%, 98%, 99% or 100% sequence identity to SEQ ID NO: 151.

In certain embodiments, the rearranged γδ TCR is endogenous to the γδ T cell.

In certain embodiment, the rearranged γδ TCR is recombinant.

In certain embodiments, the recombinant rearranged γδ TCR is activated by one or more phospho-antigens. As used, herein a “phospho-antigen” refers to a phosphorylated compound identical to a phosphorylated metabolite released by cells as by-products of the mevalonate biosynthetic pathway. Examples of phospho-antigens include, but are not limited to, isopentenyl pyrophosphate (IPP), dimethylallyl diphosphate (DMAPP), (E)-4-hydroxy-3-methyl-but-2-enylpyrophosphate (HMBPP), or chemically similar molecules. Phospho-antigens can be naturally-occurring in cells as products of metabolic processes or phospho-antigens can be caused to accumulate in cells at higher levels due to treatment with bisphosphonate chemicals. Phospho-antigens can also be a synthetic compound. “Bisphosphonate chemicals” are drugs that have a common geminal carbon atom linked to two phosphonate groups. The two phosphonate groups endow these compounds with a high affinity for divalent ions, such as calcium. Amino-bisphosphonates, such as pamidronate and zoledronic acid, inhibit the enzyme farnesyl diphosphonate (FPP) synthase in the mevalonate biosynthetic pathway inducing the accumulation of the substrates of the FPP synthase enzyme, IPP and DMAPP (van Beek et al., Biochem Biophys Res Commun, 1999; 255, 491-494). The majority of γδ T cells that respond to bisphosphonates carry genetic rearrangements resulting in utilization of the Vγ9 variable gene [TRGV9] and the Vδ2 variable gene [TRDV2]. The resulting gamma chain and delta chain TCR proteins form the Vγ9/Vδ2 TCR heterodimer. Not all bisphosphonate-responsive γδ T cells utilize the Vγ9/Vδ2 TCR pair. Other TCR variable genes can also be utilized, however Vγ9/Vδ2 are most common and present a traceable phenotype with predictable biological activity.

In certain embodiments, the recombinant rearranged γδ TCR is activated by phospho-antigens selected from isopentenyl pyrophosphate (IPP), dimethylallyl diphosphate (DMAPP), (E)-4-hydroxy-3-methyl-but-2-enylpyrophosphate [HMBPP], or chemically similar molecules. In some embodiments, the phospho-antigens can be naturally-occurring in cells as products of metabolic processes or the phospho-antigens can be caused to accumulate in cells at higher levels due to treatment with bisphosphonate chemicals. In certain embodiments, the bisphosphonate chemical is an amino-bisphosphonate, preferably the amino-bisphosphonate is zoledronic acid or salts thereof.

Certain members of the butyrophilin (BTN) protein family, such as BTN2A1, BTN3A1, BTN3A2, or BTN3A3, can be required to stimulate Vγ9Vδ2 T cells by phospho-antigens (Harly et al., Blood (2012) 120(11):2269-79; Vavassori et al., Nat. Immunol. (2013) 14(9):908-16). In certain embodiments, the activity of phospho-antigens can be through direct interaction with the γδ TCR or the activity of phospho-antigens can be through interactions with butyrophilin (BTN) proteins BTN2A1, BTN3A1, BTN3A2, or BTN3A3.

In certain embodiments, the recombinant rearranged γδ TCR is not activated by phospho-antigens.

In certain embodiments, the specific antigen recognized by the TCR is unknown or yet to be identified.

In certain embodiments, the recombinant rearranged γδ TCR enables expansion of the differentiated T cell after mitogenic stimulation. As used herein, “mitogenic stimulation” or “mitogenic activation” refers to the process of stimulating cells to proliferate by exposure to mitogens. Examples of mitogens that can be used to stimulate γδ T cells include, but are not limited to, antibodies targeting surface proteins (CD3, CD28, CD27 or other costimulatory molecules), interleukin-2 (IL-2), IL-15, IL-7, IL-21, IL-12, IL-18, phytohaemagglutinin (PHA), concanavalin A (conA), and lectins. In certain embodiments, a γδ T cell stimulating agent can be used in combination with a general T cell mitogen, for example a mitogenic cytokine such as IL-2. In certain embodiments, the γδ TCR enables expansion of the differentiated T cell after mitogenic stimulation that is dependent on the TCR. In certain embodiments, the γδ TCR enables increased expansion of the differentiated T cell after mitogenic stimulation than the T cell without the rearranged TCR locus.

In certain embodiments, the iPSC is reprogrammed from whole blood peripheral blood mononuclear cells (PBMCs). In certain embodiments, the iPSC is prepared by expanding PBMCs in the presence of an amino-bisphosphonate and interleukin 2 (IL-2) prior to incorporating reprogramming transcription factors into the peripheral blood cells to generate the iPSC.

### II. Chimeric Antigen Receptor (CAR) Expression

According to embodiments of the application, an iPSC cell or derivative cell thereof comprises (i) one or more polynucleotides encoding recombinant rearranged γδ T cell receptor (TCR); and (ii) a polynucleotide encoding a chimeric antigen receptor (CAR), such as a CAR targeting a tumor antigen.

As used herein, the term “chimeric antigen receptor” (CAR) refers to a recombinant polypeptide comprising at least an extracellular domain that binds specifically to an antigen or a target, a transmembrane domain and an intracellular signaling domain. Engagement of the extracellular domain of the CAR with the target antigen on the surface of a target cell results in clustering of the CAR and delivers an activation stimulus to the CAR-containing cell. CARs redirect the specificity of immune effector cells and trigger proliferation, cytokine production, phagocytosis and/or production of molecules that can mediate cell death of the target antigen-expressing cell in a major histocompatibility (MHC)-independent manner.

As used herein, the term “signal peptide” refers to a leader sequence at the amino-terminus (N-terminus) of a nascent CAR protein, which co-translationally or post-translationally directs the nascent protein to the endoplasmic reticulum and subsequent surface expression.

As used herein, the term “extracellular antigen binding domain,” “extracellular domain,” or “extracellular ligand binding domain” refers to the part of a CAR that is located outside of the cell membrane and is capable of binding to an antigen, target or ligand.

As used herein, the term “hinge region” or “hinge domain” refers to the part of a CAR that connects two adjacent domains of the CAR protein, i.e., the extracellular domain and the transmembrane domain of the CAR protein.

As used herein, the term “transmembrane domain” refers to the portion of a CAR that extends across the cell membrane and anchors the CAR to cell membrane.

As used herein, the term “intracellular signaling domain,” “cytoplasmic signaling domain,” or “intracellular signaling domain” refers to the part of a CAR that is located inside of the cell membrane and is capable of transducing an effector signal.

As used herein, the term “stimulatory molecule” refers to a molecule expressed by an immune cell (e.g., T cell) that provides the primary cytoplasmic signaling sequence(s) that regulate primary activation of receptors in a stimulatory way for at least some aspect of the immune cell signaling pathway. Stimulatory molecules comprise two distinct classes of cytoplasmic signaling sequence, those that initiate antigen-dependent primary activation (referred to as “primary signaling domains”), and those that act in an antigen-independent manner to provide a secondary of co-stimulatory signal (referred to as “co-stimulatory signaling domains”).

In certain embodiments, the extracellular domain comprises an antigen binding domain and/or an antigen binding fragment. The antigen binding fragment can, for example, be an antibody or antigen binding fragment thereof that specifically binds a tumor antigen. The antigen binding fragments of the application possess desirable functional properties, including but not limited to high-affinity binding to a tumor antigen.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the application can be of any of the five major classes or corresponding sub-classes. Preferably, the antibodies of the application are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the application can contain a kappa or lambda light chain constant domain. According to particular embodiments, the antibodies of the application include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to the specific tumor antigen is substantially free of antibodies that do not bind to the tumor antigen). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies of the application can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdAb), a scFv dimer (bivalent diabody), a multispecific antibody formed from a portion of an antibody comprising one or more CDRs, a camelized single domain antibody, a minibody, a nanobody, a domain antibody, a bivalent domain antibody, a light chain variable domain (VL), a variable domain (V_{H}H) of a camelid antibody, or any other antibody fragment that binds to an antigen but does not comprise a complete antibody structure. An antigen-binding fragment is capable of binding to the same antigen to which the parent antibody or a parent antibody fragment binds.

As used herein, the term “single-chain antibody” refers to a conventional single-chain antibody in the field, which comprises a heavy chain variable region and a light chain variable region connected by a short peptide of about 15 to about 20 amino acids (e.g., a linker peptide).

As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, a bispecific antibody comprises a V_{H}H having binding specificity for a first epitope, and a V_{H}H having binding specificity for a second epitope.

As used herein, an antigen binding domain or antigen binding fragment that “specifically binds to a tumor antigen” refers to an antigen binding domain or antigen binding fragment that binds a tumor antigen, with a KD of 1×10⁻⁷ M or less, preferably 1×10⁻⁸M or less, more preferably 5×10⁻⁹M or less, 1×10⁻⁹M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antigen binding domain or antigen binding fragment can be determined by using surface plasmon resonance, such as by using a biosensor system, e.g., a Biacore® system, or by using bio-layer interferometry technology, such as an Octet RED96 system.

The smaller the value of the KD of an antigen binding domain or antigen binding fragment, the higher affinity that the antigen binding domain or antigen binding fragment binds to a target antigen.

In various embodiments, antibodies or antibody fragments suitable for use in the CAR of the present disclosure include, but are not limited to, monoclonal antibodies, bispecific antibodies, multispecific antibodies, chimeric antibodies, polypeptide-Fc fusions, single-chain Fvs (scFv), single chain antibodies, Fab fragments, F(ab′) fragments, disulfide-linked Fvs (sdFv), masked antibodies (e.g., Probodies®), Small Modular ImmunoPharmaceuticals (“SMIPs™”), intrabodies, minibodies, single domain antibody variable domains, nanobodies, VHHs, diabodies, tandem diabodies (TandAb®), anti-idiotypic (anti-Id) antibodies (including, e.g., anti-Id antibodies to antigen-specific TCR), and epitope-binding fragments of any of the above. Antibodies and/or antibody fragments can be derived from murine antibodies, rabbit antibodies, human antibodies, fully humanized antibodies, camelid antibody variable domains and humanized versions, shark antibody variable domains and humanized versions, and camelized antibody variable domains.

In some embodiments, the antigen-binding fragment is an Fab fragment, an Fab′ fragment, an F(ab′)2 fragment, an scFv fragment, an Fv fragment, a dsFv diabody, a V_{H}H, a VNAR, a single-domain antibody (sdAb) or nanobody, a dAb fragment, a Fd′ fragment, a Fd fragment, a heavy chain variable region, an isolated complementarity determining region (CDR), a diabody, a triabody, or a decabody. In some embodiments, the antigen-binding fragment is an scFv fragment.

In certain embodiments, the antigen binding domain of the CAR is a single-domain antibody (sdAb), also known as a nanobody, an antibody fragment consisting of a single monomeric variable antibody domain, including heavy-chain antibodies found in camelids; the so called V_{H}H fragments. (Hamers-Casterman et al., Nature, 363, 446448 (1993); see also U.S. Pat. Nos. 5,759,808; 5,800,988; 5,840,526; and 5,874,541, hereby incorporated by reference). Cartilaginous fishes also have heavy-chain antibodies (IgNAR, ‘immunoglobulin new antigen receptor’), from which single-domain antibodies called VNAR fragments can be obtained and these can be used in the invention. An alternative approach is to split the dimeric variable domains from common immunoglobulin G (IgG) from humans or mice into monomers. Although most research into single-domain antibodies is currently based on heavy chain variable domains, nanobodies derived from light chains have also been shown to bind specifically to target epitopes and can also be employed.

Alternative scaffolds to immunoglobulin domains that exhibit similar functional characteristics, such as high-affinity and specific binding of target biomolecules, can also be used in the CARs of the present disclosure. Such scaffolds have been shown to yield molecules with improved characteristics, such as greater stability or reduced immunogenicity. Non-limiting examples of alternative scaffolds that can be used in the CAR of the present disclosure include engineered, tenascin-derived, tenascin type III domain (e.g., Centyrin™); engineered, gamma-B crystallin-derived scaffold or engineered, ubiquitin-derived scaffold (e.g., Affilins); engineered, fibronectin-derived, 10th fibronectin type III (10Fn3) domain (e.g., monobodies, AdNectins™ or AdNexins™); engineered, ankyrin repeat motif containing polypeptide (e.g., DARPins™); engineered, low-density-lipoprotein-receptor-derived, A domain (LDLR-A) (e.g., Avimers™); lipocalin (e.g., anticalins); engineered, protease inhibitor-derived, Kunitz domain (e.g., EETI-II/AGRP, BPTI/LACI-D1/ITI-D2); engineered, Protein-A-derived, Z domain (Affibodies™); Sac7d-derived polypeptides (e.g., Nanoffitins® or affitins); engineered, Fyn-derived, SH2 domain (e.g., Fynomers®); CTLD3 (e.g., Tetranectin); thioredoxin (e.g., peptide aptamer); KALBITOR®; the β-sandwich (e.g., iMab); miniproteins; C-type lectin-like domain scaffolds; engineered antibody mimics; and any genetically manipulated counterparts of the foregoing that retains its binding functionality (Worn A, Pluckthun A, J Mol Biol 305: 989-1010 (2001); Xu L et al., Chem Biol 9: 933-42 (2002); Wikman M et al., Protein Eng Des Sel 17: 455-62 (2004); Binz H et al., Nat Biolechnol 23: 1257-68 (2005); Hey T et al., Trends Biotechnol 23:514-522 (2005); Holliger P, Hudson P, Nat Biotechnol 23: 1126-36 (2005); Gill D, Damle N, Curr Opin Biotech 17: 653-8 (2006); Koide A, Koide S, Methods Mol Biol 352: 95-109 (2007); Skerra, Current Opin. in Biotech., 2007 18: 295-304; Byla P et al., J Biol Chem 285: 12096 (2010); Zoller F et al., Molecules 16: 2467-85 (2011), each of which is incorporated by reference in its entirety).

In some embodiments, the alternative scaffold is Affilin or Centyrin.

In some embodiments, the first polypeptide of the CARs of the present disclosure comprises a leader sequence. The leader sequence can be positioned at the N-terminus the extracellular tag-binding domain. The leader sequence can be optionally cleaved from the extracellular tag-binding domain during cellular processing and localization of the CAR to the cellular membrane. Any of various leader sequences known to one of skill in the art cam be used as the leader sequence. Non-limiting examples of peptides from which the leader sequence can be derived include granulocyte-macrophage colony-stimulating factor receptor (GMCSFR), FcεR, human immunoglobulin (IgG) heavy chain (HC) variable region, CD8a, or any of various other proteins secreted by T cells. In various embodiments, the leader sequence is compatible with the secretory pathway of a T cell. In certain embodiments, the leader sequence is derived from human immunoglobulin heavy chain (HC).

In some embodiments, the leader sequence is derived from GMCSFR. In one embodiment, the GMCSFR leader sequence comprises the amino acid sequence set forth in SEQ ID NO: 1, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 1.

In some embodiments, the first polypeptide of the CARs of the present disclosure comprise a transmembrane domain, fused in frame between the extracellular tag-binding domain and the cytoplasmic domain.

The transmembrane domain can be derived from the protein contributing to the extracellular binding domain, the protein contributing the signaling or co-signaling domain, or by a totally different protein. In some instances, the transmembrane domain can be selected or modified by amino acid substitution, deletions, or insertions to minimize interactions with other members of the CAR complex. In some instances, the transmembrane domain can be selected or modified by amino acid substitution, deletions, or insertions to avoid binding of proteins naturally associated with the transmembrane domain. In certain embodiments, the transmembrane domain includes additional amino acids to allow for flexibility and/or optimal distance between the domains connected to the transmembrane domain.

The transmembrane domain can be derived either from a natural or from a synthetic source. Where the source is natural, the domain can be derived from any membrane-bound or transmembrane protein. Non-limiting examples of transmembrane domains of particular use in this disclosure can be derived from (i.e. comprise at least the transmembrane region(s) of) the α, β or ζ chain of the T cell receptor (TCR), CD28, CD3 epsilon, CD45, CD4, CD5, CD8, CD8α, CD9, CD16, CD22, CD33, CD37, CD40, CD64, CD80, CD86, CD134, CD137, or CD154. Alternatively, the transmembrane domain can be synthetic, in which case it will comprise predominantly hydrophobic residues such as leucine and valine. For example, a triplet of phenylalanine, tryptophan and/or valine can be found at each end of a synthetic transmembrane domain.

In some embodiments, it will be desirable to utilize the transmembrane domain of the ζ, η or FcεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some instances, the transmembrane domain will be selected or modified by amino acid substitution to avoid binding of such domains to the transmembrane domains of the same or different surface membrane proteins to minimize interactions with other members of the receptor complex. In other cases, it will be desirable to employ the transmembrane domain of ζ, η or FcεR1γ and -β, MB1 (Igα.), B29 or CD3-γ, δ, or η, in order to retain physical association with other members of the receptor complex.

In some embodiments, the transmembrane domain is derived from CD8 or CD28. In one embodiment, the CD8 transmembrane domain comprises the amino acid sequence set forth in SEQ ID NO: 23, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 23. In one embodiment, the CD28 transmembrane domain comprises the amino acid sequence set forth in SEQ ID NO: 24, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 24.

In some embodiments, the first polypeptide of the CAR of the present disclosure comprises a spacer region between the extracellular binding domain and the transmembrane domain, wherein the binding domain, linker, and the transmembrane domain are in frame with each other.

The term “spacer region” as used herein generally means any oligo- or polypeptide that functions to link the binding domain to the transmembrane domain. A spacer region can be used to provide more flexibility and accessibility for the binding domain. A spacer region can comprise up to 300 amino acids, preferably 10 to 100 amino acids and most preferably 25 to 50 amino acids. A spacer region can be derived from all or part of naturally occurring molecules, such as from all or part of the extracellular region of CD8, CD4 or CD28, or from all or part of an antibody constant region. Alternatively, the spacer region can be a synthetic sequence that corresponds to a naturally occurring spacer region sequence, or can be an entirely synthetic spacer region sequence. Non-limiting examples of spacer regions which can be used in accordance to the disclosure include a part of human CD8a chain, partial extracellular domain of CD28, FcγRllla receptor, IgG, IgM, IgA, IgD, IgE, an Ig hinge, or functional fragment thereof. In some embodiments, additional linking amino acids are added to the spacer region to ensure that the antigen-binding domain is an optimal distance from the transmembrane domain. In some embodiments, when the spacer is derived from an Ig, the spacer can be mutated to prevent Fc receptor binding.

In some embodiments, the spacer region comprises a hinge domain. The hinge domain can be derived from CD8α, CD28, or an immunoglobulin (IgG). For example, the IgG hinge can be from IgG1, IgG2, IgG3, IgG4, IgM1, IgM2, IgA1, IgA2, IgD, IgE, or a chimera thereof.

In certain embodiments, the hinge domain comprises an immunoglobulin IgG hinge or functional fragment thereof. In certain embodiments, the IgG hinge is from IgG1, IgG2, IgG3, IgG4, IgM1, IgM2, IgA1, IgA2, IgD, IgE, or a chimera thereof. In certain embodiments, the hinge domain comprises the CH1, CH2, CH3 and/or hinge region of the immunoglobulin. In certain embodiments, the hinge domain comprises the core hinge region of the immunoglobulin. The term “core hinge” can be used interchangeably with the term “short hinge” (a.k.a “SH”). Non-limiting examples of suitable hinge domains are the core immunoglobulin hinge regions include EPKSCDKTHTCPPCP (SEQ ID NO: 57) from IgG1, ERKCCVECPPCP (SEQ ID NO: 58) from IgG2, ELKTPLGDTTHTCPRCP(EPKSCDTPPPCPRCP)₃ (SEQ ID NO: 59) from IgG3, and ESKYGPPCPSCP (SEQ ID NO: 60) from IgG4 (see also Wypych et al., JBC 2008 283(23): 16194-16205, which is incorporated herein by reference in its entirety for all purposes). In certain embodiments, the hinge domain is a fragment of the immunoglobulin hinge.

In some embodiments, the hinge domain is derived from CD8 or CD28. In one embodiment, the CD8 hinge domain comprises the amino acid sequence set forth in SEQ ID NO: 21, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 21. In one embodiment, the CD28 hinge domain comprises the amino acid sequence set forth in SEQ ID NO: 22, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 22.

In some embodiments, the transmembrane domain and/or hinge domain is derived from CD8 or CD28. In some embodiments, both the transmembrane domain and hinge domain are derived from CD8. In some embodiments, both the transmembrane domain and hinge domain are derived from CD28.

In certain aspects, the first polypeptide of CARs of the present disclosure comprise a cytoplasmic domain, which comprises at least one intracellular signaling domain. In some embodiments, cytoplasmic domain also comprises one or more co-stimulatory signaling domains.

The cytoplasmic domain is responsible for activation of at least one of the normal effector functions of the host cell (e.g., T cell) in which the CAR has been placed in. The term “effector function” refers to a specialized function of a cell. Effector function of a T cell, for example, can be cytolytic activity or helper activity including the secretion of cytokines. Thus, the term “signaling domain” refers to the portion of a protein which transduces the effector function signal and directs the cell to perform a specialized function. While usually the entire signaling domain is present, in many cases it is not necessary to use the entire chain. To the extent that a truncated portion of the intracellular signaling domain is used, such truncated portion can be used in place of the intact chain as long as it transduces the effector function signal. The term intracellular signaling domain is thus meant to include any truncated portion of the signaling domain sufficient to transduce the effector function signal.

Non-limiting examples of signaling domains which can be used in the CARs of the present disclosure include, e.g., signaling domains derived from DAP10, DAP12, Fc epsilon receptor I γ chain (FCER1G), FcR β, CD3δ, CD3ε, CD3γ, CD3εCD2, CD5, CD22, CD226, CD66d, CD79A, and CD79B.

In some embodiments, the cytoplasmic domain comprises a CD3ζ signaling domain. In one embodiment, the CD3ζ signaling domain comprises the amino acid sequence set forth in SEQ ID NO: 6, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 6.

In some embodiments, the cytoplasmic domain further comprises one or more co-stimulatory signaling domains. In some embodiments, the one or more co-stimulatory signaling domains are derived from CD28, 41BB, IL2Rb, CD40, OX40 (CD134), CD80, CD86, CD27, ICOS, NKG2D, DAP10, DAP12, 2B4 (CD244), BTLA, CD30, GITR, CD226, CD79A, and HVEM.

In one embodiment, the co-stimulatory signaling domain is derived from 41BB. In one embodiment, the 41BB co-stimulatory signaling domain comprises the amino acid sequence set forth in SEQ ID NO: 8, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 8.

In one embodiment, the co-stimulatory signaling domain is derived from IL2Rb. In one embodiment, the IL2Rb co-stimulatory signaling domain comprises the amino acid sequence set forth in SEQ ID NO: 9, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 9.

In one embodiment, the co-stimulatory signaling domain is derived from CD40. In one embodiment, the CD40 co-stimulatory signaling domain comprises the amino acid sequence set-forth in SEQ ID NO: 10, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 10.

In one embodiment, the co-stimulatory signaling domain is derived from OX40. In one embodiment, the OX40 co-stimulatory signaling domain comprises the amino acid sequence set forth in SEQ ID NO: 11, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 11.

In one embodiment, the co-stimulatory signaling domain is derived from CD80. In one embodiment, the CD80 co-stimulatory signaling domain comprises the amino acid sequence set forth in SEQ ID NO: 12, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 12.

In one embodiment, the co-stimulatory signaling domain is derived from CD86. In one embodiment, the CD86 co-stimulatory signaling domain comprises the amino acid sequence set forth in SEQ ID NO: 13, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 13.

In one embodiment, the co-stimulatory signaling domain is derived from CD27. In one embodiment, the CD27 co-stimulatory signaling domain comprises the amino acid sequence set forth in SEQ ID NO: 14, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 14.

In one embodiment, the co-stimulatory signaling domain is derived from ICOS. In one embodiment, the ICOS co-stimulatory signaling domain comprises the amino acid sequence set forth in SEQ ID NO: 15, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 15.

In one embodiment, the co-stimulatory signaling domain is derived from NKG2D. In one embodiment, the NKG2D co-stimulatory signaling domain comprises the amino acid sequence set forth in SEQ ID NO: 16, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 16.

In one embodiment, the co-stimulatory signaling domain is derived from DAP10. In one embodiment, the DAP10 co-stimulatory signaling domain comprises the amino acid sequence set forth in SEQ ID NO: 17, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 17.

In one embodiment, the co-stimulatory signaling domain is derived from DAP12. In one embodiment, the DAP12 co-stimulatory signaling domain comprises the amino acid sequence set forth in SEQ ID NO: 18, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 18.

In one embodiment, the co-stimulatory signaling domain is derived from 2B4 (CD244). In one embodiment, the 2B4 (CD244) co-stimulatory signaling domain comprises the amino acid sequence set forth in SEQ ID NO: 19, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 19.

In some embodiments, the CAR of the present disclosure comprises one costimulatory signaling domains. In some embodiments, the CAR of the present disclosure comprises two or more costimulatory signaling domains. In certain embodiments, the CAR of the present disclosure comprises two, three, four, five, six or more costimulatory signaling domains.

In some embodiments, the signaling domain(s) and costimulatory signaling domain(s) can be placed in any order. In some embodiments, the signaling domain is upstream of the costimulatory signaling domains. In some embodiments, the signaling domain is downstream from the costimulatory signaling domains. In the cases where two or more costimulatory domains are included, the order of the costimulatory signaling domains could be switched.

Non-limiting exemplary CAR regions and sequences are provided in Table 1.

TABLE 1

| | | | |
|---|---|---|---|
| CAR | | | SEQ ID |
| regions | Sequence | UniProt Id | NO |

CD19 CAR:

| | | | |
|---|---|---|---|
| GMCSFR | MLLLVTSLLLCELPHPAFLLIP | | 1 |
| Signal | | | |
| Peptide | | | |
| FMC63 VH | EVKLQESGPGLVAPSQSLSVTCTVSGVSLPDYG | | 2 |
| | VSWIRQPPRKGLEWLGVIWGSETTYYNSALKSR | | |
| | LTIIKDNSKSQVFLKMNSLQTDDTAIYYCAKHY | | |
| | YYGGSYAMDYWGQGTSVTVSS | | |
| Whitlow | GSTSGSGKPGSGEGSTKG | | 3 |
| Linker | | | |
| FMC63 VL | DIQMTQTTSSLSASLGDRVTISCRASQDISKYLN | | 4 |
| | WYQQKPDGTVKLLIYHTSRLHSGVPSRFSGSGS | | |
| | GTDYSLTISNLEQEDIATYFCQQGNTLPYTFGG | | |
| | GTKLEIT | | |
| CD28 | IEVMYPPPYLDNEKSNGTIIHVKGKHLCPSPLFP | P10747-1 | 5 |
| (AA 114- | GPSKPFWVLVVVGGVLACYSLLVTVAFIIFWV | | |
| 220) | RSKRSRLLHSDYMNMTPRRPGPTRKHYQPYAP | | |
| | PRDFAAYRS | | |
| CD3-zeta | RVKFSRSADAPAYQQGQNQLYNELNLGRREEY | P20963-3 | 6 |
| isoform 3 | DVLDKRRGRDPEMGGKPRRKNPQEGLYNELQ | | |
| (AA 52-163) | KDKMAEAYSEIGMKGERRRGKGHDGLYQGLS | | |
| | TATKDTYDALHMQALPPR | | |
| FMC63 | EVKLQESGPGLVAPSQSLSVTCTVSGVSLPDYG | | 7 |
| scFV | VSWIRQPPRKGLEWLGVIWGSETTYYNSALKS | | |
| | RLTIIKDNSKSQVFLKMNSLQTDDTAIYYCAKH | | |
| | YYYGGSYAMDYWGQGTSVTVSSGSTSGSGKP | | |
| | GSGEGSTKGDIQMTQTTSSLSASLGDRVTISCR | | |
| | ASQDISKYLNWYQQKPDGTVKLLIYHTSRLHS | | |
| | GVPSRFSGSGSGTDYSLTISNLEQEDIATYFCQQ | | |
| | GNTLPYTFGGGTKLEIT | | |

Sisnallins Domains:

| | | | |
|---|---|---|---|
| 41BB | KRGRKKLLYIFKQPFMRPVQTTQEEDGCSCRFP | Q07011 | 8 |
| (AA214- | EEEEGGCEL | | |
| 255) | | | |
| IL2Rb | NCRNTGPWLKKVLKCNTPDPSKFFSQLSSEHG | P14784 | 9 |
| (AA 266- | GDVQKWLSSPFPSSSFSPGGLAPEISPLEVLERD | | |
| 551) | KVTQLLPLNTDAYLSLQELQGQDPTHLV | | |
| CD40 | KKVAKKPTNKAPHPKQEPQEINFPDDLPGSNTA | P25942 | 10 |
| (AA216- | APVQETLHGCQPVTQEDGKESRISVQERQ | | |
| 277) | | | |
| OX40 | ALYLLRRDQRLPPDAHKPPGGGSFRTPIQEEQA | P43489 | 11 |
| (AA 236- | DAHSTLAKI | | |
| 277) | | | |
| CD80 | TYCFAPRCRERRRNERLRRESVRPV | P33681 | 12 |
| (AA 264- | | | |
| 288) | | | |
| CD86 | KWKKKKRPRNSYKCGTNTMEREESEQTKKRE | P42081 | 13 |
| (AA269-329) | KIHIPERSDEAQRVFKSSKTSSCDKSDTCF | | |
| CD27 | QRRKYRSNKGESPVEPAEPCHYSCPREEEGSTIP | P26842 | 14 |
| (AA213- | IQEDYRKPEPACSP | | |
| 260) | | | |
| ICOS | CWLTKKKYSSSVHDPNGEYMFMRAVNTAKKS | Q9Y6W8 | 15 |
| (AA 162- | RLTDVTL | | |
| 199) | | | |
| NKG2D | MGWIRGRRSRHSWEMSEFHNYNLDLKKSDF | P26718 | 16 |
| (AA 1-51) | STRWQKQRCPVVKSKCRENAS | | |
| DAP10 | LCARPRRSPAQEDGKVYINMPGRG | Q9UBK5 | 17 |
| (AA 70-93) | | | |
| DAP12 | YFLGRLVPRGRGAAEAATRKQRITETESPYQEL | O54885 | 18 |
| (AA 62-113) | QGQRSDVYSDLNTQRPYYK | | |
| 2B4/CD244 | WRRKRKEKQSETSPKEFLTIYEDVKDLKTRRN | Q9BZW8 | 19 |
| (AA251- | HEQEQTFPGGGSTIYSMIQSQSSAPTSQEPAYTL | | |
| 370) | YSLIQPSRKSGSRKRNHSPSFNSTIYEVIGKSQP | | |
| | KAQNPARLSRKELENFDVYS | | |
| CD3-zeta | RVKFSRSADAPAYQQGQNQLYNELNLGRREEY | P20963-3 | 6 |
| isoform 3 | DVLDKRRGRDPEMGGKPRRKNPQEGLYNELQ | | |
| (AA 52-163) | KDKMAEAYSEIGMKGERRRGKGHDGLYQGLS | | |
| | TATKDTYDALHMQALPPR | | |
| CD28 | RSKRSRLLHSDYMNMTPRRPGPTRKHYQPYAP | P10747-1 | 20 |
| (AA 180- | PRDFAAYRS | | |
| 220) | | | |

Spacer/Hinge:

| | | | |
|---|---|---|---|
| CD8 | TTTPAPRPPTPAPTIASQPLSLRPEACRPAAGGA | P01732 | 21 |
| (AA 136- | VHTRGLDFACDIY | | |
| 182) | | | |
| CD28 | IEVMYPPPYLDNEKSNGTIIHVKGKHLCPSPLFP | P10747-1 | 22 |
| (AA 114- | GPSKP | | |
| 151) | | | |

Transmembrane:

| | | | |
|---|---|---|---|
| CD8 | IYIWAPLAGTCGVLLLSLVIT | P01732 | 23 |
| (AA 183- | | | |
| 203) | | | |
| CD28 | FWVLVVVGGVLACYSLLVTVAFIIFWV | P10747-1 | 24 |
| (AA 153- | | | |
| 179) | | | |

Linkers:

| | | |
|---|---|---|
| Whitlow | GSTSGSGKPGSGEGSTKG | 3 |
| Linker | | |
| (G₄S)₃ | GGGGSGGGGSGGGGS | 25 |
| Linker 3 | GGSEGKSSGSGSESKSTGGS | 26 |
| Linker 4 | GGGSGGGS | 27 |
| Linker 5 | GGGSGGGSGGGS | 28 |
| Linker 6 | GGGSGGGSGGGSGGGS | 29 |
| Linker 7 | GGGSGGGSGGGSGGGSGGGS | 30 |
| Linker 8 | GGGGSGGGGSGGGGSGGGGS | 31 |
| Linker 9 | GGGGSGGGGSGGGGSGGGGSGGGGS | 32 |
| Linker 10 | IRPRAIGGSKPRVA | 33 |
| Linker 11 | GKGGSGKGGSGKGGS | 34 |
| Linker 12 | GGKGSGGKGSGGKGS | 35 |
| Linker 13 | GGGKSGGGKSGGGKS | 36 |
| Linker 14 | GKGKSGKGKSGKGKS | 37 |
| Linker 15 | GGGKSGGKGSGKGGS | 38 |
| Linker 16 | GKPGSGKPGSGKPGS | 39 |
| Linker 17 | GKPGSGKPGSGKPGSGKPGS | 40 |
| Linker 18 | GKGKSGKGKSGKGKSGKGKS | 41 |
| Linker 19 | STAGDTHLGGEDFD | 42 |
| Linker 20 | GEGGSGEGGSGEGGS | 43 |
| Linker 21 | GGEGSGGEGSGGEGS | 44 |
| Linker 22 | GEGESGEGESGEGES | 45 |
| Linker 23 | GGGESGGEGSGEGGS | 46 |
| Linker 24 | GEGESGEGESGEGESGEGES | 47 |
| Linker 25 | GSTSGSGKPGSGEGSTKG | 48 |
| Linker 26 | PRGASKSGSASQTGSAPGS | 49 |
| Linker 27 | GTAAAGAGAAGGAAAGAAG | 50 |
| Linker 28 | GTSGSSGSGSGGSGSGGGG | 51 |
| Linker 29 | GKPGSGKPGSGKPGSGKPGS | 52 |
| Linker 30 | GSGS | 53 |
| Linker 31 | APAPAPAPAP | 54 |
| Linker 32 | APAPAPAPAPAPAPAPAPAP | 55 |
| Linker 33 | AEAAAKEAAAKEAAAAKEAAAAKEAAAAKA | 56 |
| | AA | |

In some embodiments, the antigen-binding domain of the second polypeptide binds to an antigen. The antigen-binding domain of the second polypeptide can bind to more than one antigen or more than one epitope in an antigen. For example, the antigen-binding domain of the second polypeptide can bind to two, three, four, five, six, seven, eight or more antigens. As another example, the antigen-binding domain of the second polypeptide can bind to two, three, four, five, six, seven, eight or more epitopes in the same antigen.

The choice of antigen-binding domain may depend upon the type and number of antigens that define the surface of a target cell. For example, the antigen-binding domain can be chosen to recognize an antigen that acts as a cell surface marker on target cells associated with a particular disease state. In certain embodiments, the CARs of the present disclosure can be genetically modified to target a tumor antigen of interest by way of engineering a desired antigen-binding domain that specifically binds to an antigen (e.g., on a tumor cell). Non-limiting examples of cell surface markers that can act as targets for the antigen-binding domain in the CAR of the disclosure include those associated with tumor cells or autoimmune diseases.

In some embodiments, the antigen-binding domain binds to at least one tumor antigen or autoimmune antigen.

In some embodiments, the antigen-binding domain binds to at least one tumor antigen. In some embodiments, the antigen-binding domain binds to two or more tumor antigens. In some embodiments, the two or more tumor antigens are associated with the same tumor. In some embodiments, the two or more tumor antigens are associated with different tumors. In some embodiments, the antigen-binding domain binds to at least one autoimmune antigen. In some embodiments, the antigen-binding domain binds to two or more autoimmune antigens. In some embodiments, the two or more autoimmune antigens are associated with the same autoimmune disease. In some embodiments, the two or more autoimmune antigens are associated with different autoimmune diseases.

In some embodiments, the tumor antigen is associated with glioblastoma, ovarian cancer, cervical cancer, head and neck cancer, liver cancer, prostate cancer, pancreatic cancer, renal cell carcinoma, bladder cancer, or hematologic malignancy. Non-limiting examples of tumor antigen associated with glioblastoma include HER2, EGFRvIII, EGFR, CD133, PDGFRA, FGFR1, FGFR3, MET, CD70, ROBO1 and IL13Rα2. Non-limiting examples of tumor antigens associated with ovarian cancer include FOLR1, FSHR, MUC16, MUC1, Mesothelin, CA125, EpCAM, EGFR, PDGFRα, Nectin-4, and B7H4. Non-limiting examples of the tumor antigens associated with cervical cancer or head and neck cancer include GD2, MUC1, Mesothelin, HER2, and EGFR. Non-limiting examples of tumor antigen associated with liver cancer include Claudin 18.2, GPC-3, EpCAM, cMET, and AFP. Non-limiting examples of tumor antigens associated with hematological malignancies include CD22, CD79 (CD79a and/or CD79b), BCMA, GPRC5D, SLAM F7, CD33, CLL1, CD123, and CD70. Non-limiting examples of tumor antigens associated with bladder cancer include Nectin-4 and SLITRK6.

Additional examples of antigens that can be targeted by the antigen-binding domain include, but are not limited to, alpha-fetoprotein, A3, antigen specific for A33 antibody, Ba 733, BrE3-antigen, carbonic anhydrase EX, CD1, CD1a, CD3, CD5, CD15, CD16, CD19, CD20, CD21, CD22, CD23, CD25, CD30, CD33, CD38, CD45, CD74, CD79a, CD80, CD123, CD138, colon-specific antigen-p (CSAp), CEA (CEACAM5), CEACAM6, CSAp, EGFR, EGP-I, EGP-2, Ep-CAM, EphA1, EphA2, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le-Y, macrophage inhibition factor (MIF), MAGE, MUC2, MUC3, MUC4, NCA66, NCA95, NCA90, antigen specific for PAM-4 antibody, placental growth factor, p53, prostatic acid phosphatase, PSA, PSMA, RS5, 5100, TAC, TAG-72, tenascin, TRAIL receptors, Tn antigen, Thomson-Friedenreich antigens, tumor necrosis antigens, VEGF, ED-B fibronectin, 17-1A-antigen, an angiogenesis marker, an oncogene marker or an oncogene product.

In one embodiment, the antigen targeted by the antigen-binding domain is CD19. In one embodiment, the antigen-binding domain comprises an anti-CD19 scFv. In one embodiment, the anti-CD19 scFv comprises a heavy chain variable region (VH) comprising the amino acid sequence set forth in SEQ ID NO: 2, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 2. In one embodiment, the anti-CD19 scFv comprises a light chain variable region (VL) comprising the amino acid sequence set forth in SEQ ID NO: 4, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 4. In one embodiment, the anti-CD19 scFv comprises the amino acid sequence set forth in SEQ ID NO: 7, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 7.

In some embodiments, the antigen is associated with an autoimmune disease or disorder. Such antigens can be derived from cell receptors and cells which produce “self”-directed antibodies. In some embodiments, the antigen is associated with an autoimmune disease or disorder such as Rheumatoid arthritis (RA), multiple sclerosis (MS), Sjögren's syndrome, Systemic lupus erythematosus, sarcoidosis, Type 1 diabetes mellitus, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Myasthenia gravis, Hashimoto's thyroiditis, Graves' disease, chronic inflammatory demyelinating polyneuropathy, Guillain-Barre syndrome, Crohn's disease or ulcerative colitis.

In some embodiments, autoimmune antigens that can be targeted by the CAR disclosed herein include but are not limited to platelet antigens, myelin protein antigen, Sm antigens in snRNPs, islet cell antigen, Rheumatoid factor, and anticitrullinated protein. citrullinated proteins and peptides such as CCP-1, CCP-2 (cyclical citrullinated peptides), fibrinogen, fibrin, vimentin, fillaggrin, collagen I and II peptides, alpha-enolase, translation initiation factor 4G1, perinuclear factor, keratin, Sa (cytoskeletal protein vimentin), components of articular cartilage such as collagen II, IX, and XI, circulating serum proteins such as RFs (IgG, IgM), fibrinogen, plasminogen, ferritin, nuclear components such as RA33/hnRNP A2, Sm, eukaryotic translation elogation factor 1 alpha 1, stress proteins such as HSP-65, -70, -90, BiP, inflammatory/immune factors such as B7-H1, IL-1 alpha, and IL-8, enzymes such as calpastatin, alpha-enolase, aldolase-A, dipeptidyl peptidase, osteopontin, glucose-6-phosphate isomerase, receptors such as lipocortin 1, neutrophil nuclear proteins such as lactoferrin and 25-35 kD nuclear protein, granular proteins such as bactericidal permeability increasing protein (BPI), elastase, cathepsin G, myeloperoxidase, proteinase 3, platelet antigens, myelin protein antigen, islet cell antigen, rheumatoid factor, histones, ribosomal P proteins, cardiolipin, vimentin, nucleic acids such as dsDNA, ssDNA, and RNA, ribonuclear particles and proteins such as Sm antigens (including but not limited to SmD's and SmB7B), U1RNP, A2/B1 hnRNP, Ro (SSA), and La (SSB) antigens.

In various embodiments, the scFv fragment used in the CAR of the present disclosure can include a linker between the VH and VL domains. The linker can be a peptide linker and can include any naturally occurring amino acid. Exemplary amino acids that can be included into the linker are Gly, Ser Pro, Thr, Glu, Lys, Arg, Ile, Leu, His and The. The linker should have a length that is adequate to link the VH and the VL in such a way that they form the correct conformation relative to one another so that they retain the desired activity, such as binding to an antigen. The linker can be about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. Exemplary linkers that can be used are Gly rich linkers, Gly and Ser containing linkers, Gly and Ala containing linkers, Ala and Ser containing linkers, and other flexible linkers.

In one embodiment, the linker is a Whitlow linker. In one embodiment, the Whitlow linker comprises the amino acid sequence set forth in SEQ ID NO: 3, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 3. In another embodiment, the linker is a (G₄S)₃ linker. In one embodiment, the (G₄S)₃ linker comprises the amino acid sequence set forth in SEQ ID NO: 25, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 25.

Other linker sequences can include portions of immunoglobulin hinge area, CL or CH1 derived from any immunoglobulin heavy or light chain isotype. Exemplary linkers that can be used include any of SEQ ID NOs: 26-56 in Table 1. Additional linkers are described for example in Int. Pat. Publ. No. WO2019/060695, incorporated by reference herein in its entirety.

### III. Artificial Cell Death Polypeptide Safety Switch

According to certain embodiments of the application, an iPSC cell or a derivative cell thereof comprises an exogenous polynucleotide encoding an artificial cell death polypeptide.

As used herein, the term “an artificial cell death polypeptide” refers to an engineered protein designed to prevent potential toxicity or otherwise adverse effects of a cell therapy. The artificial cell death polypeptide could mediate induction of apoptosis, inhibition of protein synthesis, DNA replication, growth arrest, transcriptional and post-transcriptional genetic regulation and/or antibody-mediated depletion. In some instance, the artificial cell death polypeptide is activated by an exogenous molecule, e.g. an antibody, anti-viral drug, or radioisotopic conjugate drugs, that when activated, triggers apoptosis and/or cell death of a therapeutic cell. In certain embodiments, the mechanism of action of the artificial cell death polypeptide is metabolic, dimerization-inducing or therapeutic monoclonal antibody-mediated.

In certain embodiments, artificial cell death polypeptide is an inactivated cell surface receptor that comprises an epitope specifically recognized by an antibody, particularly a monoclonal antibody, which is also referred to herein as a monoclonal antibody-specific epitope. When expressed by iPSCs or derivative cells thereof, the inactivated cell surface receptor is signaling inactive or significantly impaired, but can still be specifically recognized by an antibody. The specific binding of the antibody to the inactivated cell surface receptor enables the elimination of the iPSCs or derivative cells thereof by ADCC and/or ADCP mechanisms, as well as, direct killing with antibody drug conjugates with toxins or radionuclides.

In certain embodiments, the inactivated cell surface receptor comprises an epitope that is selected from epitopes specifically recognized by an antibody, including but not limited to, ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, polatuzumab vedotin, ranibizumab, tocilizumab, trastuzumab, vedolizumab, adalimumab, belimumab, canakinumab, denosumab, golimumab, ipilimumab, ofatumumab, panitumumab, daratumumab, or ustekinumab.

Epidermal growth factor receptor, also known as EGFR, ErbB1 and HER1, is a cell-surface receptor for members of the epidermal growth factor family of extracellular ligands. As used herein, “truncated EGFR,” “tEGFR,” “short EGFR” or “sEGFR” refers to an inactive EGFR variant that lacks the EGF-binding domains and the intracellular signaling domains of the EGFR. An exemplary tEGFR variant contains residues 322-333 of domain 2, all of domains 3 and 4 and the transmembrane domain of the native EGFR sequence containing the cetuximab binding epitope. Expression of the tEGFR variant on the cell surface enables cell elimination by an antibody that specifically binds to the tEGFR, such as cetuximab (Erbitux®), as needed. Due to the absence of the EGF-binding domains and intracellular signaling domains, tEGFR is inactive when expressed by iPSCs or derivative cell thereof.

An exemplary inactivated cell surface receptor of the application comprises a tEGFR variant. In certain embodiments, expression of the inactivated cell surface receptor in an engineered immune cell expressing a chimeric antigen receptor (CAR) induces cell suicide of the engineered immune cell when the cell is contacted with an anti-EGFR antibody. Methods of using inactivated cell surface receptors are described in WO2019/070856, WO2019/023396, WO2018/058002, the disclosure of which is incorporated herein by reference. For example, a subject who has previously received an engineered immune cell of the present disclosure that comprises a heterologous polynucleotide encoding an inactivated cell surface receptor comprising a tEGFR variant can be administered an anti-EGFR antibody in an amount effective to ablate in the subject the previously administered engineered immune cell.

In certain embodiments, the anti-EGFR antibody is cetuximab, matuzumab, necitumumab or panitumumab, preferably the anti-EGFR antibody is cetuximab.

In certain embodiments, the tEGFR variant comprises or consists of an amino acid sequence at least 90%, such as at least 90%, 91%, 82%, 93%, 94%, 95%, 96%, 97%, 98%, 99% or 100%, identical to SEQ ID NO: 71, preferably the amino acid sequence of SEQ ID NO: 71.

In some embodiments, the inactivated cell surface receptor comprises one or more epitopes of CD79b, such as an epitope specifically recognized by polatuzumab vedotin. In certain embodiments, the CD79b epitope comprises or consists of an amino acid sequence at least 90%, such as at least 90%, 91%, 82%, 93%, 94%, 95%, 96%, 97%, 98%, 99% or 100%, identical to SEQ ID NO: 78, preferably the amino acid sequence of SEQ ID NO: 78.

In some embodiments, the inactivated cell surface receptor comprises one or more epitopes of CD20, such as an epitope specifically recognized by rituximab. In certain embodiments, the CD20 epitope comprises or consists of an amino acid sequence at least 90%, such as at least 90%, 91%, 82%, 93%, 94%, 95%, 96%, 97%, 98%, 99% or 100%, identical to SEQ ID NO: 80, preferably the amino acid sequence of SEQ ID NO: 80.

In some embodiments, the inactivated cell surface receptor comprises one or more epitopes of Her 2 receptor or ErbB, such as an epitope specifically recognized by trastuzumab. In certain embodiments, the monoclonal antibody-specific epitope comprises or consists of an amino acid sequence at least 90%, such as at least 90%, 91%, 82%, 93%, 94%, 95%, 96%, 97%, 98%, 99% or 100%, identical to SEQ ID NO: 82, preferably the amino acid sequence of SEQ ID NO: 82.

In some embodiments the inactivated cell surface receptor further comprises a cytokine.

In some embodiments, an inactivated cell surface receptor further comprises a hinge domain. In some embodiments, the hinge domain is derived from CD8. In one embodiment, the CD8 hinge domain comprises the amino acid sequence set forth in SEQ ID NO: 21, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 21.

In certain embodiments, an inactivated cell surface receptor further comprises a transmembrane domain. In some embodiments, the transmembrane domain is derived from CD8. In one embodiment, the CD8 transmembrane domain comprises the amino acid sequence set forth in SEQ ID NO: 23, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 23.

In certain embodiment, an inactivated cell surface receptor comprises one or more epitopes specifically recognized by an antibody in its extracellular domain, a transmembrane region and a cytoplasmic domain. In some embodiments, the inactivated cell surface receptor further comprises a hinge region between the epitope(s) and the transmembrane region. In some embodiments, the inactivated cell surface receptor comprises more than one epitopes specifically recognized by an antibody, the epitopes can have the same or different amino acid sequences, and the epitopes can be linked together via a peptide linker, such as a flexible peptide linker have the sequence of (GGGGS)n, wherein n is an integer of 1-8 (SEQ ID NO: 25). In some embodiments, the inactivated cell surface receptor further comprises a cytokine. In certain embodiments, the cytokine is in the cytoplasmic domain of the inactivated cell surface receptor. Preferably, the cytokine is operably linked to the epitope(s) specifically recognized by an antibody, directly or indirectly, via an autoprotease peptide sequence, such as those described herein. In some embodiments, the cytokine is indirectly linked to the epitope(s) by connecting to the transmembrane region via the autoprotease peptide sequence.

In other embodiments, the artificial cell death polypeptide is a viral enzyme that is recognized by an antiviral drug. In certain embodiments, the viral enzyme is a herpes simplex virus thymidine kinase (HSV-tk). In certain embodiments, the HSV-tk comprises or consists of an amino acid sequence at least 90%, such as at least 90%, 91%, 82%, 93%, 94%, 95%, 96%, 97%, 98%, 99% or 100%, identical to SEQ ID NO: 143, preferably the amino acid sequence of SEQ ID NO: 143. This enzyme phosphorylates the nontoxic prodrug ganciclovir, which then becomes phosphorylated by endogenous kinases to GCV-triphosphate, causing chain termination and single-strand breaks upon incorporation into DNA, thereby killing dividing cells. In certain embodiments, expression of the viral enzyme in an engineered immune cell expressing a chimeric antigen receptor (CAR) induces cell death of the engineered immune cell when the cell is contacted with an antiviral drug. In certain embodiments, the antiviral drug is ganciclovir.

In certain embodiments, the artificial cell death polypeptide comprises an antigen targeted by a small molecule compound. In certain embodiments, the antigen is a truncated prostate-specific membrane antigen (PSMA) polypeptide as described in Intl. Pat. Applications WO2015143029A1 and WO2018187791A1, the disclosures of which are incorporated by reference into the present application in entirety. In certain embodiments, the prostate-specific membrane antigen (PSMA) polypeptide comprises or consists of an amino acid sequence at least 90%, such as at least 90%, 91%, 82%, 93%, 94%, 95%, 96%, 97%, 98%, 99% or 100%, identical to SEQ ID NO: 144, preferably the amino acid sequence of SEQ ID NO: 144. In certain embodiments, expression of truncated PSMA in an engineered immune cell expressing a chimeric antigen receptor (CAR) induces cell death of the engineered immune cell when the cell is contacted with a radioisotopic conjugate drug that binds to PSMA via a small peptide. PSMA-targeting compounds are described in WO2010/108125, the disclosure of which is incorporated herein by reference.

In certain embodiments, the artificial cell death polypeptide comprises a herpes simplex virus thymidine kinase (HSV-tk) fused to a prostate-specific membrane antigen (PSMA) polypeptide via a linker. In certain embodiments, the linker comprises an amino acid sequence of SEQ ID NO: 48. In certain embodiments, the artificial cell death polypeptide comprises an amino acid sequence at least 90%, such as at least 90%, 91%, 82%, 93%, 94%, 95%, 96%, 97%, 98%, 99% or 100%, identical to SEQ ID NO: 145, preferably the amino acid sequence of SEQ ID NO: 145.

In certain embodiments, the artificial cell death polypeptide comprises a herpes simplex virus thymidine kinase (HSV-tk) and a prostate-specific membrane antigen (PSMA) polypeptide operably linked by an autoprotease peptide sequence. In certain embodiments, the autoprotease peptide is a thosea asigna virus 2A (T2A) peptide. In certain embodiments, the artificial cell death polypeptide comprises an amino acid sequence at least 90%, such as at least 90%, 91%, 82%, 93%, 94%, 95%, 96%, 97%, 98%, 99% or 100%, identical to SEQ ID NO: 146, preferably the amino acid sequence of SEQ ID NO: 146.

In certain embodiments, the artificial polypeptide comprises a prostate-specific membrane antigen (PSMA) polypeptide and a cluster of differentiation 24 (CD24) polypeptide operably linked by an autoprotease peptide sequence. In certain embodiments, the autoprotease peptide is a thosea asigna virus 2A (T2A) peptide. In certain embodiments, the artificial cell death polypeptide comprises an amino acid sequence at least 90%, such as at least 90%, 91%, 82%, 93%, 94%, 95%, 96%, 97%, 98%, 99% or 100%, identical to SEQ ID NO: 147, preferably the amino acid sequence of SEQ ID NO: 147.

### IV. HLA Expression

In one aspect, MHC I and/or MHC II knock-out and/or knock down can be incorporated in the cells for use in “allogeneic” cell therapies, in which cells are harvested from a subject, modified to knock-out or knock-down, e.g., disrupt, B2M, TAP 1, TAP 2, Tapasin, RFXANK, CIITA, RFX5 and RFXAP gene expression, and then returned to a different subject. Knocking out or knocking down the B2M, TAP 1, TAP 2, Tapasin, RFXANK, CIITA, RFX5 and RFXAP genes genes as described herein can: (1) prevent GvH response; (2) prevent HvG response; and/or (3) improve T cell safety and efficacy. Accordingly, in certain embodiments, a presently disclosed invention comprises independently knocking out and/or knocking down one or more genes selected from the group consisting of B2M, TAP 1, TAP 2, Tapasin, RFXANK, CIITA, RFX5 and RFXAP genes in a T cell. In certain embodiments, a presently disclosed method comprises independently knocking out and/or knocking down two genes selected from the group consisting of B2M, TAP 1, TAP 2, Tapasin, RFXANK, CIITA, RFX5 and RFXAP genes in a T cell, in particular, B2M and CIITA to achieve class I and II HLA disruption. In certain embodiments, an iPSC or derivative cell thereof of the application can be further modified by introducing an exogenous polynucleotide encoding one or more proteins related to immune evasion, such as non-classical HLA class I proteins (e.g., HLA-E and HLA-G). In particular, disruption of the B2M gene eliminates surface expression of all MHC class I molecules, leaving cells vulnerable to lysis by NK cells through the “missing self” response. Exogenous HLA-E expression can lead to resistance to NK-mediated lysis (Gornalusse et al., Nat Biotechnol. 2017; 35(8): 765-772).

In certain embodiments, the iPSC or derivative cell thereof comprises an exogenous polypeptide encoding at least one of a human leukocyte antigen E (HLA-E) and human leukocyte antigen G (HLA-G). In a particular embodiment, the HLA-E comprises an amino acid sequence at least 90%, such as at least 90%, 91%, 82%, 93%, 94%, 95%, 96%, 97%, 98%, 99% or 100%, identical to SEQ ID NO: 65, preferably the amino acid sequence of SEQ ID NO: 65. In a particular embodiment, the HLA-G comprises an amino acid sequence at least 90%, such as at least 90%, 91%, 92%, 93%, 94%, 95%, 96%, 97%, 98%, 99% or 100%, identical to SEQ ID NO: 68, preferably the amino acid sequence of SEQ ID NO: 68.

In certain embodiments, the exogenous polynucleotide encodes a polypeptide comprising a signal peptide operably linked to a mature B2M protein that is fused to an HLA-E via a linker. In a particular embodiment, the exogenous polypeptide comprises an amino acid sequence at least sequence at least 90%, such as at least 90%, 91%, 82%, 93%, 94%, 95%, 96%, 97%, 98%, 99% or 100%, identical to SEQ ID NO: 66.

In other embodiments, the exogenous polynucleotide encodes a polypeptide comprising a signal peptide operably linked to a mature B2M protein that is fused to an HLA-G via a linker. In a particular embodiment, the exogenous polypeptide comprises an amino acid sequence at least sequence at least 90%, such as at least 90%, 91%, 82%, 93%, 94%, 95%, 96%, 97%, 98%, 99% or 100%, identical to SEQ ID NO: 69.

### V. Other Optional Genome Edits

In certain embodiments, a cell of the application further comprises an exogenous polynucleotide encoding interleukin 15 (IL-15) and/or interleukin (IL-15) receptor or a variant or truncation thereof. As used herein “Interleukin-15” or “IL-15” refers to a cytokine that regulates T and NK cell activation and proliferation. A “functional portion” (“biologically active portion”) of IL-15 refers to a portion of IL-15 that retains one or more functions of full length or mature IL-15. Such functions include the promotion of NK cell survival, regulation of NK cell and T cell activation and proliferation as well as the support of NK cell development from hematopoietic stem cells. As will be appreciated by those of skill in the art, the sequence of a variety of IL-15 molecules are known in the art. In certain embodiments, the IL-15 is a wild-type IL-15. In certain embodiments, the IL-15 is a human IL-15.

In another embodiment, the cell of the application further comprises an exogenous polynucleotide encoding a non-naturally occurring variant of FcγRIII (CD16), for example, hnCD16 (see, e.g., Zhu et al., Blood 2017, 130:4452, the contents of which are incorporated herein in their entirety by reference). As used herein, the term “hnCD16a” refers to a high affinity, non-cleavable variant of CD16 (a low-affinity Fcγ receptor involved in antibody-dependent cellular cytotoxicity (ADCC). Typically, CD16 is cleaved during ADCC by proteases whereas the hnCD16 CAR does not undergo this cleavage and thus sustains an ADCC signal longer. In some embodiments, the hnCD 16 is as disclosed in Blood 2016 128:3363, the entire contents of which is expressly incorporated herein by reference.

In another embodiment, a cell of the application further comprises an exogenous polynucleotide encoding interleukin 12 (IL-12) or interleukin 21 (IL-21) or a variant thereof.

In another embodiment, a cell of the application further comprises an exogenous polynucleotide encoding leukocyte surface antigen cluster of differentiation CD47 (CD47) as an NK inhibitory modality to overcome host-versus-graft immunoreactivity for allogeneic applications. As used herein, the term “CD47,” also sometimes referred to as “integrin associated protein” (IAP), refers to a transmembrane protein that in humans is encoded by the CD47gene. CD47 belongs to the immunoglobulin superfamily, partners with membrane integrins, and also binds the ligands thrombospondin-1 (TSP-1) and signal-regulatory protein alpha (SIRPa). CD47 acts as a signal to macrophages that allows CD47-expressing cells to escape macrophage attack. See, e.g., Deuse-T, et al., Nature Biotechnology 2019 37: 252-258, the entire contents of which are incorporated herein by reference.

In another embodiment, a cell of the application further comprises an exogeneous polynucleotide encoding a constitutively active IL-7 receptor or variant thereof. IL-7 has a critical role in the development and maturation of T cells. It promotes the generation of naïve and central memory T cell subsets and regulates their homeostasis. It has previously been reported that IL-7 prolonged the survival time of tumor-specific T cells in vivo. Cancer Medicine. 2014; 3(3):550-554. In previous studies, it has been reported that a constitutively activated IL-7 receptor (C7R) could result in IL-7 signaling in the absence of a ligand or with the existence of gamma chain (γc) of a coreceptor. Shum et al, Cancer Discovery. 2017; 7(11):1238-1247. Insertion of a transmembrane domain such as cysteine and/or proline resulted in the homodimerization of IL-7Rα. Upon the formation of a homodimer, cross-phosphorylation of JAK1/JAK1 activates STATS, thereby activating the downstream signaling of IL-7. Constructs for such constitutively activated IL-7 receptor (C7R) compositions are disclosed in WO2018/038945, the contents of which are hereby incorporated by reference into the present application.

In another embodiment, a cell of the application further comprises an exogenous polynucleotide encoding one or more imaging or reporter proteins, such as PSMA or HSV-tk. For example, the cell can contain an exogeneous polynucleotide encoding prostate-specific membrane antigen (PSMA) as an imaging reporter in accordance with the disclosures of WO2015/143029 and WO2018/187791, the disclosures of which are incorporated herein by reference.

In one embodiment of the above described cell, the genomic editing at one or more selected sites can comprise insertions of one or more exogenous polynucleotides encoding other additional artificial cell death polypeptides proteins, targeting modalities, receptors, signaling molecules, transcription factors, pharmaceutically active proteins and peptides, drug target candidates, or proteins promoting engraftment, trafficking, homing, viability, self-renewal, persistence, and/or survival of the genome-engineered iPSCs or derivative cells thereof.

In some embodiments, the exogenous polynucleotides for insertion are operatively linked to (1) one or more exogenous promoters comprising CMV, EFla, PGK, CAG, UBC, or other constitutive, inducible, temporal-, tissue-, or cell type-specific promoters; or (2) one or more endogenous promoters comprised in the selected sites comprising AAVS1, CCR5, ROSA26, collagen, HTRP, Hll, beta-2 microglobulin, GAPDH, TCR or RUNX1, or other locus meeting the criteria of a genome safe harbor. In some embodiments, the genome-engineered iPSCs generated using the above method comprise one or more different exogenous polynucleotides encoding proteins comprising caspase, thymidine kinase, cytosine deaminase, B-cell CD20, ErbB2 or CD79b wherein when the genome-engineered iPSCs comprise two or more suicide genes, the suicide genes are integrated in different safe harbor locus comprising AAVS1, CCR5, ROSA26, collagen, HTRP, Hll, Hll, beta-2 microglobulin, GAPDH, TCR or RUNX1. Other exogenous polynucleotides encoding proteins can include those encoding PET reporters, homeostatic cytokines, and inhibitory checkpoint inhibitory proteins such as PD1, PD-L1, and CTLA4 as well as proteins that target the CD47/signal regulatory protein alpha (SIRPα) axis. In some other embodiments, the genome-engineered iPSCs generated using the method provided herein comprise in/del at one or more endogenous genes associated with targeting modality, receptors, signaling molecules, transcription factors, drug target candidates, immune response regulation and modulation, or proteins suppressing engraftment, trafficking, homing, viability, self-renewal, persistence, and/or survival of the iPSCs or derivative cells thereof.

In addition, the modified γδ cells can exhibit one or more edits in their genome that results in a loss-of-function in a target gene. A loss-of-function of a target gene is characterized by a decrease in the expression of a target gene based on a genomic modification, e.g., an RNA-guided nuclease-mediated cut in the target gene that results in an inactivation, or in diminished expression or function, of the encoded gene product. Examples of genes that can be targeted for loss of function include B2M, PD-1, CISH, CIITA, HLA class II histocompatibility alpha chain genes (e.g. HLA-DQA1, HLA-DRA, HLA-DPAL HLA-DMA-HLA-DQA2 and or HLA-DOA), HLA Class II histocompatabilty beta chain genes (e.g HLA-DMB, HLA-DOB, HLA-DPB1, HLA-DQB1, HLA-DQB2, HLA-DQB3, HLA-DRB1, HLADRB3, HLA-DRB4, and/or HLA-DRB5), CD32B, CTLA4, NKG2A, BIM, CCR5, CCR7, CD96, CDK8, CXCR3, EP4 (PGE2 RECEPTOR), Fas, GITR, IL1R8, KIRDL1, KIR2DL1-3, LAG3, SOCS genes, Sortilin, TIM3, TRAC, RAG1, RAG2 and NLRC5.

The modified cells of the application can exhibit any of the edits described, as well as any combination of such edits described.

### VI. Targeted Genome Editing at Selected Locus in iPSCs

According to embodiments of the application, one or more of the exogenous polynucleotides are integrated at one or more loci on the chromosome of an iPSC.

Genome editing, or genomic editing, or genetic editing, as used interchangeably herein, is a type of genetic engineering in which DNA is inserted, deleted, and/or replaced in the genome of a targeted cell. Targeted genome editing (interchangeable with “targeted genomic editing” or “targeted genetic editing”) enables insertion, deletion, and/or substitution at pre-selected sites in the genome. When an endogenous sequence is deleted or disrupted at the insertion site during targeted editing, an endogenous gene comprising the affected sequence can be knocked-out or knocked-down due to the sequence deletion or disruption. Therefore, targeted editing can also be used to disrupt endogenous gene expression with precision. Similarly used herein is the term “targeted integration,” referring to a process involving insertion of one or more exogenous sequences at pre-selected sites in the genome, with or without deletion of an endogenous sequence at the insertion site.

Targeted editing can be achieved either through a nuclease-independent approach, or through a nuclease-dependent approach. In the nuclease-independent targeted editing approach, homologous recombination is guided by homologous sequences flanking an exogenous polynucleotide to be inserted, through the enzymatic machinery of the host cell.

Alternatively, targeted editing could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration.”

Available endonucleases capable of introducing specific and targeted DSBs include, but not limited to, zinc-finger nucleases (ZFN), transcription activator-like effector nucleases (TALEN) and RNA-guided CRISPR (Clustered Regular Interspaced Short Palindromic Repeats) systems. Additionally, DICE (dual integrase cassette exchange) system utilizing phiC31 and Bxbl integrases is also a promising tool for targeted integration.

ZFNs are targeted nucleases comprising a nuclease fused to a zinc finger DNA binding domain. By a “zinc finger DNA binding domain” or “ZFBD” it is meant a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A “designed” zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A “selected” zinc finger domain is a domain not found in nature whose production results primarily from an empirical process such as phage display, interaction trap or hybrid selection. ZFNs are described in greater detail in U.S. Pat. Nos. 7,888,121 and 7,972,854, the complete disclosures of which are incorporated herein by reference. The most recognized example of a ZFN in the art is a fusion of the Fokl nuclease with a zinc finger DNA binding domain.

A TALEN is a targeted nuclease comprising a nuclease fused to a TAL effector DNA binding domain. By “transcription activator-like effector DNA binding domain”, “TAL effector DNA binding domain”, or “TALE DNA binding domain” it is meant the polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in U.S. Patent Application No. 2011/0145940, which is herein incorporated by reference. The most recognized example of a TALEN in the art is a fusion polypeptide of the Fokl nuclease to a TAL effector DNA binding domain.

Additional examples of targeted nucleases suitable for the present application include, but not limited to Spol1, Bxbl, phiC3 1, R4, PhiBTl, and Wp/SPBc/TP90l-1, whether used individually or in combination.

Other non-limiting examples of targeted nucleases include naturally occurring and recombinant nucleases; CRISPR related nucleases from families including cas, cpf, cse, csy, csn, csd, cst, csh, csa, csm, and cmr; restriction endonucleases; meganucleases; homing endonucleases, and the like. As an example, CRISPR/Cas9 requires two major components: (1) a Cas9 endonuclease and (2) the crRNA-tracrRNA complex. When co-expressed, the two components form a complex that is recruited to a target DNA sequence comprising PAM and a seeding region near PAM. The crRNA and tracrRNA can be combined to form a chimeric guide RNA (gRNA) to guide Cas9 to target selected sequences. These two components can then be delivered to mammalian cells via transfection or transduction. As another example, CRISPR/Cpf1 comprises two major components: (1) a CPf1 endonuclease and (2) a crRNA. When co-expressed, the two components form a ribobnucleoprotein (RNP) complex that is recruited to a target DNA sequence comprising PAM and a seeding region near PAM. The crRNA can be combined to form a chimeric guide RNA (gRNA) to guide Cpf1 to target selected sequences. These two components can then be delivered to mammalian cells via transfection or transduction.

MAD7 is an engineered Cas12a variant originating from the bacterium Eubacterium rectale that has a preference for 5′-TTTN-3′ and 5′-CTTN-3′ PAM sites and does not require a tracrRNA. See, for example, PCT Publication No. 2018/236548, the disclosure of which is incorporated herein by reference.

DICE mediated insertion uses a pair of recombinases, for example, phiC31 and Bxbl, to provide unidirectional integration of an exogenous DNA that is tightly restricted to each enzymes' own small attB and attP recognition sites. Because these target att sites are not naturally present in mammalian genomes, they must be first introduced into the genome, at the desired integration site. See, for example, U.S. Application Publication No. 2015/0140665, the disclosure of which is incorporated herein by reference.

One aspect of the present application provides a construct comprising one or more exogenous polynucleotides for targeted genome integration. In one embodiment, the construct further comprises a pair of homologous arm specific to a desired integration site, and the method of targeted integration comprises introducing the construct to cells to enable site specific homologous recombination by the cell host enzymatic machinery. In another embodiment, the method of targeted integration in a cell comprises introducing a construct comprising one or more exogenous polynucleotides to the cell, and introducing a ZFN expression cassette comprising a DNA-binding domain specific to a desired integration site to the cell to enable a ZFN-mediated insertion. In yet another embodiment, the method of targeted integration in a cell comprises introducing a construct comprising one or more exogenous polynucleotides to the cell, and introducing a TALEN expression cassette comprising a DNA-binding domain specific to a desired integration site to the cell to enable a TALEN-mediated insertion. In another embodiment, the method of targeted integration in a cell comprises introducing a construct comprising one or more exogenous polynucleotides to the cell, introducing a Cpf1 expression cassette, and a gRNA comprising a guide sequence specific to a desired integration site to the cell to enable a Cpf1-mediated insertion. In another embodiment, the method of targeted integration in a cell comprises introducing a construct comprising one or more exogenous polynucleotides to the cell, introducing a Cas9 expression cassette, and a gRNA comprising a guide sequence specific to a desired integration site to the cell to enable a Cas9-mediated insertion. In still another embodiment, the method of targeted integration in a cell comprises introducing a construct comprising one or more att sites of a pair of DICE recombinases to a desired integration site in the cell, introducing a construct comprising one or more exogenous polynucleotides to the cell, and introducing an expression cassette for DICE recombinases, to enable DICE-mediated targeted integration.

Sites for targeted integration include, but are not limited to, genomic safe harbors, which are intragenic or extragenic regions of the human genome that, theoretically, are able to accommodate predictable expression of newly integrated DNA without adverse effects on the host cell or organism. In certain embodiments, the genome safe harbor for the targeted integration is one or more loci of genes selected from the group consisting of AAVS1, CCR5, ROSA26, collagen, HTRP, Hll, GAPDH, TCR and RUNX1 genes.

In other embodiments, the site for targeted integration is selected for deletion or reduced expression of an endogenous gene at the insertion site. As used herein, the term “deletion” with respect to expression of a gene refers to any genetic modification that abolishes the expression of the gene. Examples of “deletion” of expression of a gene include, e.g., a removal or deletion of a DNA sequence of the gene, an insertion of an exogenous polynucleotide sequence at a locus of the gene, and one or more substitutions within the gene, which abolishes the expression of the gene.

Genes for target deletion include, but are not limited to, genes of major histocompatibility complex (MHC) class I and MHC class II proteins. Multiple MHC class I and class II proteins must be matched for histocompatibility in allogeneic recipients to avoid allogeneic rejection problems. “MHC deficient”, including MHC-class I deficient, or MHC-class II deficient, or both, refers to cells that either lack, or no longer maintain, or have reduced level of surface expression of a complete MHC complex comprising a MHC class I protein heterodimer and/or a MHC class II heterodimer, such that the diminished or reduced level is less than the level naturally detectable by other cells or by synthetic methods. MHC class I deficiency can be achieved by functional deletion of any region of the MHC class I locus (chromosome 6p2l), or deletion or reducing the expression level of one or more MHC class-I associated genes including, not being limited to, beta-2 microglobulin (B2M) gene, TAP 1 gene, TAP 2 gene and Tapasin genes. For example, the B2M gene encodes a common subunit essential for cell surface expression of all MHC class I heterodimers. B2M null cells are MHC-I deficient. MHC class II deficiency can be achieved by functional deletion or reduction of MHC-II associated genes including, not being limited to, RFXANK, CIITA, RFX5 and RFXAP. CIITA is a transcriptional coactivator, functioning through activation of the transcription factor RFX5 required for class II protein expression. CIITA null cells are MHC-II deficient. In certain embodiments, one or more of the exogenous polynucleotides are integrated at one or more loci of genes selected from the group consisting of B2M, TAP 1, TAP 2, Tapasin, RFXANK, CIITA, RFX5 and RFXAP genes to thereby delete or reduce the expression of the gene(s) with the integration.

Other genes for target deletion include, but are not limited to, recombination-activating genes 1 and 2 (RAG1 and RAG2). RAG1 and RAG2 encode parts of a protein complex that initiate V(D)J recombination by introducing double-strand breaks at the border between a recombination signal sequence (RSS) and a coding segment. Deletion or reducing the expression level of the RAG1/RAG2 genes prevents additional TCR rearrangement in the cell, thus preventing unexpected generation of auto-reactive TCR (Minagawa et al., Cell Stem Cell. 2018 Dec. 6; 23(6):850-858).

In certain embodiments, the exogenous polynucleotides are integrated at one or more loci on the chromosome of the cell, preferably the one or more loci are of genes selected from the group consisting of AAVS1, CCR5, ROSA26, collagen, HTRP, Hl 1, GAPDH, RUNX1, B2M, TAPI, TAP2, Tapasin, NLRC5, CIITA, RFXANK, CIITA, RFX5, RFXAP, TRAC, TRBC1, TRBC2, RAG1, RAG2, NKG2A, NKG2D, CD38, CIS, CBL-B, SOCS2, PD1, CTLA4, LAG3, TIM3, or TIGIT genes, provided at least one of the one or more loci is of a MHC gene, such as a gene selected from the group consisting of B2M, TAP 1, TAP 2, Tapasin, RFXANK, CIITA, RFX5 and RFXAP genes. In some embodiments, one or more of the exogenous polynucleotides are integrated at a CD38 locus, thereby resulting in a deletion or reduced expression of the CD38 gene. Preferably, the one or more exogenous polynucleotides are integrated at a locus of an MHC class-I associated gene, such as a beta-2 microglobulin (B2M) gene, TAP 1 gene, TAP 2 gene or Tapasin gene; and at a locus of an MHC-II associated gene, such as a RFXANK, CIITA, RFX5, RFXAP, or CIITA gene; and optionally further at a locus of a safe harbor gene selected from the group consisting of AAVS1, CCR5, ROSA26, collagen, HTRP, Hll, GAPDH, TCR and RUNX1 genes. More preferably, the one or more of the exogenous polynucleotides are integrated at the loci of CIITA, AAVS1 and B2M genes.

In certain embodiments, (i) the exogenous polynucleotide encoding the chimeric antigen receptor (CAR) is integrated at a locus of AAVS1 gene; (ii) the exogenous polypeptide encoding the artificial cell death polypeptide is integrated at a locus of CIITA gene; and (iii) the exogenous polypeptide encoding the human leukocyte antigen E (HLA-E) and/or human leukocyte antigen G (HLA-G) is integrated at a locus of B2M gene; wherein integrations of the exogenous polynucleotides delete or reduce expression of CIITA and B2M genes.

### VII. Derivative Cells

In another aspect, the invention relates to a cell derived from differentiation of an iPSC of the application, a derivative cell. As described above, the genomic edits introduced into the iPSC cell are retained in the derivative cell. In certain embodiments of the derivative cell obtained from iPSC differentiation, the derivative cell is a T cell. In other embodiments, the derivative cell is a CD34+ hematopoietic progenitor cell (HPC).

In certain embodiments, the application provides a CD34+ hematopoietic progenitor cell (HPC) derived from an induced pluripotent stem cell (iPSC) comprising: (i) an exogenous polynucleotide encoding a chimeric antigen receptor (CAR); and (ii) one or more polynucleotides encoding a rearranged γδ T cell receptor (TCR), wherein the rearranged γδ TCR supports differentiation of the iPSC to a T cell. In certain embodiments, the application provides a CD34+ hematopoietic progenitor cell (HPC) derived from an induced pluripotent stem cell (iPSC) comprising: (i) an exogenous polynucleotide encoding a chimeric antigen receptor (CAR), (ii) an exogenous polynucleotide encoding an artificial cell death polypeptide; (iii) a deletion or reduced expression of one or more of B2M, TAP 1, TAP 2, Tapasin, RFXANK, CIITA, RFX5, RAG1/RAG2 and RFXAP genes; and (iv) one or more polynucleotides encoding a rearranged γδ T cell receptor (TCR), wherein the rearranged γδ TCR supports differentiation of the iPSC to a T cell.

In certain embodiments, the application provides a T cell comprising (i) an exogenous polynucleotide encoding a chimeric antigen receptor (CAR); and (ii) one or more polynucleotides encoding a rearranged γδ T cell receptor (TCR), wherein the rearranged γδ TCR supports differentiation of the iPSC to a T cell. In certain embodiments, the application provides a CD34+ hematopoietic progenitor cell (HPC) derived from an induced pluripotent stem cell (iPSC) comprising: (i) an exogenous polynucleotide encoding a chimeric antigen receptor (CAR), (ii) an exogenous polynucleotide encoding an artificial cell death polypeptide; (iii) a deletion or reduced expression of one or more of B2M, TAP 1, TAP 2, Tapasin, RFXANK, CIITA, RFX5 and RFXAP genes; (iv) a deletion or reduced expression of the RAG1/RAG2 genes; and (v) one or more polynucleotides encoding a rearranged γδ T cell receptor (TCR), wherein the rearranged γδ TCR supports differentiation of the iPSC to a T cell.

In certain embodiments, the iPSC is reprogrammed from whole peripheral blood mononuclear cells (PBMCs).

In certain embodiments, the iPSC is reprogrammed from peripheral blood CD34+ hematopoietic progenitor cells (HPCs).

In certain embodiments, the iPSC is reprogrammed from peripheral blood αβ T cells. In certain embodiments, the iPSC is reprogrammed from peripheral blood γδ T cells. In certain embodiments, the iPSC is reprogrammed from a γδ T cell and the rearranged γδ TCR is endogenous to the γδ T cell.

In certain embodiments, the γδ TCR is recombinant.

In certain embodiments, a rearranged γδ TCR enables increased expansion of the T cell differentiated from the iPSC after mitogenic stimulation than a T cell without the rearranged TCR.

In certain embodiments, one or more polynucleotides encoding a rearranged γδ TCR comprise a γ TCR variable gene selected from a group consisting of TRGV1-5, TRGV5P, TRGV8-11 and TRGVA genes; a γ TCR joining gene selected from the group consisting of TRGJ1, TRGJ2, TRGJP, TRGJP1 and TRGJP2 genes; and a γ TCR constant gene selected from the group consisting of TRGC1 and TRGC2 genes.

In certain embodiments, one or more polynucleotides encoding a rearranged γδ TCR comprise a δ TCR variable gene selected from the group consisting of TRDV1-3 genes; a δ TCR diversity gene selected from the group consisting of TRDD1, TRDD2 and TRDD3 genes; a δ TCR joining gene selected from the group consisting of TRDJ1, TRDJ2, TRDJ3 and TRDJ4; and a δ TCR constant gene TRDC

In certain embodiments, the recombinant rearranged γδ TCR is activated by phospho-antigens selected from isopentenyl pyrophosphate (IPP), dimethylallyl diphosphate (DMAPP), (E)-4-hydroxy-3-methyl-but-2-enylpyrophosphate (HMBPP), or chemically similar molecules, wherein the phospho-antigens might be naturally-occurring in cells as part of normal metabolic processes or wherein the phospho-antigens might be caused to accumulate in cells at higher levels due to treatment with bisphosphonate chemicals, wherein the activity of phospho-antigens can be through direct interaction with the γδ TCR or wherein the activity of phospho-antigens can be through interactions with butyrophilin (BTN) proteins BTN2A1, BTN3A1, BTN3A2, or BTN3A3.

In certain embodiments, the recombinant rearranged γδ TCR is not activated by phospho-antigens and the specific antigen recognized by the TCR is unknown.

In certain embodiments, the T cell further comprising an exogenous polynucleotide encoding a human leukocyte antigen E (HLA-E) and/or human leukocyte antigen G (HLA-G).

In certain embodiments, one or more of the exogenous polynucleotides are integrated at one or more loci on the chromosome of the cell selected from the group consisting of AAVS1, CCR5, ROSA26, collagen, HTRP, Hl 1, GAPDH, RUNX1, B2M, TAPI, TAP2, Tapasin, NLRC5, CIITA, RFXANK, CIITA, RFX5, RFXAP, TRAC, TRBC1, TRBC2, RAG1, RAG2, NKG2A, NKG2D, CD38, CIS, CBL-B, SOCS2, PD1, CTLA4, LAG3, TIM3, or TIGIT genes, provided at least one of the exogenous polynucleotides is integrated at a locus of a gene selected from the group consisting of B2M, TAP 1, TAP 2, Tapasin, RFXANK, CIITA, RFX5 and RFXAP genes to thereby result in a deletion or reduced expression of the gene. In certain embodiments, one or more of the exogenous polynucleotides are integrated at the loci of the CIITA, AAVS1 and B2M genes. In some embodiments, one or more of the exogenous polynucleotides are integrated at a CD38 locus, thereby resulting in a deletion or reduced expression of the CD38 gene.

In certain embodiments, the exogenous polynucleotide encoding the chimeric antigen receptor (CAR) is integrated at a locus of AAVS1 gene; the exogenous polypeptide encoding the artificial cell death polypeptide is integrated at a locus of CIITA gene; and the exogenous polypeptide encoding the human leukocyte antigen E (HLA-E) and/or human leukocyte antigen G (HLA-G) is integrated at a locus of B2M gene; wherein integration of the exogenous polynucleotides deletes or reduces expression of CIITA and B2M.

Also provided is a T cell comprising: (i) an exogenous polynucleotide encoding a chimeric antigen receptor (CAR) having the amino acid sequence of SEQ ID NO: 61;

(ii) an exogenous polynucleotide encoding an artificial cell death polypeptide comprising an apoptosis-inducing domain having the amino acid sequence of SEQ ID NO: 71;
(iii) one or more polynucleotides encoding a rearranged T cell receptor (TCR) locus comprising a γ TCR having the amino acid sequence of SEQ ID NO: 133, 135, or 150 and a δ TCR having the amino acid sequence of SEQ ID NO: 134, 136, or 151; and
(iv) optionally, an exogenous polynucleotide encoding a human leukocyte antigen E (HLA-E) having the amino acid sequence of SEQ ID NO: 66;
wherein one or more of the exogenous polynucleotides are integrated at loci of AAVS1, CIITA and B2M genes, to thereby delete or reduce expression of CIITA and B2M.

Also provided is a method of manufacturing the T cell of the application, comprising differentiating an iPSC cell of the application under conditions for cell differentiation to thereby obtain the T cell.

An iPSC of the application can be differentiated by any method known in the art. Exemplary methods are described in U.S. Pat. Nos. 8,372,642, 8,574,179, U.S. Ser. No. 10/100,282, U.S. Ser. No. 10/865,381, WO2010/099539, WO2012/109208, WO2017/070333, WO2017/070337, WO2018/067836, WO2018/195175, WO2020/061256, WO2017/179720, WO2016/010148, and WO2018/048828, each of which are herein incorporated by reference in its entirety. The differentiation protocol can use feeder cells or can be feeder-free. As used herein, “feeder cells” or “feeders” are terms describing cells of one type that are co-cultured with cells of a second type to provide an environment in which the cells of the second type can grow, expand, or differentiate, as the feeder cells provide stimulation, growth factors and nutrients for the support of the second cell type.

Notch signaling, in particular, plays a key role in driving precursor cells towards a T cell fate. In the human thymus, the Notch family proteins DLL1, DLL4, and Jag2 (expressed by stromal cells in the thymus) signal through the receptor Notch1 (expressed by early thymocytes). In certain embodiments, the differentiation protocol comprises culturing cells in medium comprising recombinant Delta-like protein 4 (DLL4). In some embodiments, the recombinant DLL4 is a variant DLL4. Non-limiting exemplary DLL4 variants and sequences are provided in Table 2. In certain embodiments, the differentiation protocol comprises culturing cells in medium comprising recombinant Jagged 2 (JAG2). In further embodiments, the recombinant proteins are immobilized using polydopamine in the presence or absence of Protein G coating to facilitate proper orientation of the recombinant proteins.

TABLE 2

| | | SEQ ID |
|---|---|---|
| Name | Description | NO: |
| DLL4-Fc Fusion 1 | Wild type human DLL4, N-terminus | 137 |
| | through EGF5 as Fc fusion protein | |
| DLL4-Fc Fusion 2 | Wild type human DLL4, N-terminus | 138 |
| | through EGF2 as Fc fusion protein | |
| DLL4-Fc Fusion 3 | Wild type human DLL4, Full ECD | 139 |
| | as Fc fusion protein | |
| DLL4-Fc Fusion 4 | Variant human DLL4 (G28S, F107L, | 140 |
| | L206P), N-terminus through EGF5 | |
| | as Fc fusion protein | |
| DLL4-Fc Fusion 5 | Variant human DLL4, N-terminus | 141 |
| | through EGF2 as Fc fusion protein | |
| DLL4-Fc Fusion 6 | Variant human DLL4, Full ECD as | 142 |
| | Fc fusion protein | |

Extracellular Domain (ECD);
Epidermal growth factor (EGF) repeat;
N = amino terminus

### VIII. Polynucleotides, Vectors, and Host Cells

(1) Nucleic Acids Encoding a CAR

In another general aspect, the invention relates to an isolated nucleic acid encoding a chimeric antigen receptor (CAR) useful for an invention according to embodiments of the application. It will be appreciated by those skilled in the art that the coding sequence of a CAR can be changed (e.g., replaced, deleted, inserted, etc.) without changing the amino acid sequence of the protein. Accordingly, it will be understood by those skilled in the art that nucleic acid sequences encoding CARs of the application can be altered without changing the amino acid sequences of the proteins.

In certain embodiments, the isolated nucleic acid encodes a CAR targeting CD19. In a particular embodiment, the isolated nucleic acid encoding the CAR comprises a polynucleotide sequence at least 90%, such as at least 90%, 91%, 92%, 93%, 94%, 95%, 96%, 97%, 98% or 100%, identical to SEQ ID NO: 62, preferably the polynucleotide sequence of SEQ ID NO: 62.

In another general aspect, the application provides a vector comprising a polynucleotide sequence encoding a CAR useful for an invention according to embodiments of the application. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of a CAR in the cell. Conventional cloning techniques or artificial gene synthesis can be used to generate a recombinant expression vector according to embodiments of the application.

In a particular aspect, the application provides vectors for targeted integration of a CAR useful for an invention according to embodiments of the application. In certain embodiments, the vector comprises an exogenous polynucleotide having, in the 5′ to 3′ order, (a) a promoter; (b) a polynucleotide sequence encoding a CAR according to an embodiment of the application; and (c) a terminator/polyadenylation signal.

In certain embodiments, the promoter is a CAG promoter. In certain embodiments, the CAG promoter comprises the polynucleotide sequence at least 90%, such as at least 90%, 91%, 92%, 93%, 94%, 95%, 96%, 97%, 98% or 100%, identical to SEQ ID NO: 63. Other promoters can also be used, examples of which include, but are not limited to, EFla, UBC, CMV, SV40, PGK1, and human beta actin.

In certain embodiments, the terminator/polyadenylation signal is a SV40 signal. In certain embodiments, the SV40 signal comprises the polynucleotide sequence at least 90%, such as at least 90%, 91%, 92%, 93%, 94%, 95%, 96%, 97%, 98% or 100%, identical to SEQ ID NO: 64. Other terminator sequences can also be used, examples of which include, but are not limited to, BGH, hGH, and PGK.

In certain embodiments, the polynucleotide sequence encoding a CAR comprises the polynucleotide sequence at least 90%, such as at least 90%, 91%, 92%, 93%, 94%, 95%, 96%, 97%, 98% or 100%, identical to SEQ ID NO: 62.

In some embodiment, the vector further comprises a left homology arm and a right homology arm flanking the exogenous polynucleotide. As used herein, “left homology arm” and “right homology arm” refers to a pair of nucleic acid sequences that flank an exogenous polynucleotide and facilitate the integration of the exogenous polynucleotide into a specified chromosomal locus. Sequences of the left and right arm homology arms can be designed based on the integration site of interest. In some embodiment, the left or right arm homology arm is homologous to the left or right side sequence of the integration site.

In certain embodiments, the left homology arm comprises the polynucleotide sequence at least 90%, such as at least 90%, 91%, 92%, 93%, 94%, 95%, 96%, 97%, 98% or 100%, identical to SEQ ID NO: 80. In certain embodiments, the right homology arm comprises the polynucleotide sequence at least 90%, such as at least 90%, 91%, 92%, 93%, 94%, 95%, 96%, 97%, 98% or 100%, identical to SEQ ID NO: 81.

In a particular embodiment, the vector comprises a polynucleotide sequence at least 85%, such as at least 85%, 86%, 87%, 88%, 89%, 90%, 91%, 92%, 93%, 94%, 95%, 96%, 97%, 98% or 100%, identical to SEQ ID NO: 82, preferably the polynucleotide sequence of SEQ ID NO: 82.

(2) Nucleic Acids Encoding an Inactivated Cell Surface Receptor

In another general aspect, the invention relates to an isolated nucleic acid encoding an inactivated cell surface receptor useful for an invention according to embodiments of the application. It will be appreciated by those skilled in the art that the coding sequence of an inactivated cell surface receptor can be changed (e.g., replaced, deleted, inserted, etc.) without changing the amino acid sequence of the protein. Accordingly, it will be understood by those skilled in the art that nucleic acid sequences encoding an inactivated cell surface receptor of the application can be altered without changing the amino acid sequences of the proteins.

In certain embodiments, an isolated nucleic acid encodes any inactivated cell surface receptor described herein, such as that comprises a monoclonal antibody-specific epitope, and a cytokine, wherein the monoclonal antibody-specific epitope and the cytokine are operably linked by an autoprotease peptide sequence.

In some embodiments, the isolated nucleic acid encodes an inactivated cell surface receptor comprising an epitope specifically recognized by an antibody, such as ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, polatuzumab vedotin, ranibizumab, tocilizumab, trastuzumab, vedolizumab, adalimumab, belimumab, canakinumab, denosumab, golimumab, ipilimumab, ofatumumab, panitumumab, daratumumab, or ustekinumab.

In certain embodiments, the isolated nucleic acid encodes an inactivated cell surface receptor having a truncated epithelial growth factor (tEGFR) variant. Preferably, the inactivated cell surface receptor comprises an epitope specifically recognized by cetuximab, matuzumab, necitumumab or panitumumab, preferably cetuximab.

In certain embodiments, the isolated nucleic acid encodes an inactivated cell surface receptor having one or more epitopes of CD79b, such as an epitope specifically recognized by polatuzumab vedotin.

In certain embodiments, the isolated nucleic acid encodes an inactivated cell surface receptor having one or more epitopes of CD20, such as an epitope specifically recognized by rituximab.

In certain embodiments, the isolated nucleic acid encodes an inactivated cell surface receptor having one or more epitopes of Her 2 receptor, such as an epitope specifically recognized by trastuzumab

In certain embodiments, the autoprotease peptide sequence is porcine tesehovirus-1 2A (P2A).

In certain embodiments, the truncated epithelial growth factor (tEGFR) variant consists of an amino acid sequence having at least 90%, 91%, 92%, 93%, 94%, 95%, 96%, 97%, 98%, 99% or 100% sequence identity to the amino acid sequence of SEQ ID NO: 71.

In certain embodiments, the monoclonal antibody-specific epitope specifically recognized by polatuzumab vedotin consists of an amino acid sequence at least 90%, such as at least 90%, 91%, 82%, 93%, 94%, 95%, 96%, 97%, 98%, 99% or 100%, identical to SEQ ID NO: 74.

In certain embodiments, the monoclonal antibody-specific epitope specifically recognized by rituximab consists of an amino acid sequence at least 90%, such as at least 90%, 91%, 82%, 93%, 94%, 95%, 96%, 97%, 98%, 99% or 100%, identical to SEQ ID NO: 75.

In certain embodiments, the monoclonal antibody-specific epitope specifically recognized by trastuzumab consists of an amino acid sequence at least 90%, such as at least 90%, 91%, 82%, 93%, 94%, 95%, 96%, 97%, 98%, 99% or 100%, identical to SEQ ID NO: 76.

In certain embodiments, the autoprotease peptide has an amino acid sequence having at least 90%, 91%, 92%, 93%, 94%, 95%, 96%, 97%, 98%, 99% or 100% sequence identity to the amino acid sequence of SEQ ID NO: 72.

In another general aspect, the application provides a vector comprising a polynucleotide sequence encoding an inactivated cell surface receptor useful for an invention according to embodiments of the application. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of an inactivated cell surface receptor in the cell. Conventional cloning techniques or artificial gene synthesis can be used to generate a recombinant expression vector according to embodiments of the application.

In a particular aspect, the application provides a vector for targeted integration of an inactivated cell surface receptor useful for an invention according to embodiments of the application. In certain embodiments, the vector comprises an exogenous polynucleotide having, in the 5′ to 3′ order, (a) a promoter; (b) a polynucleotide sequence encoding an inactivated cell surface receptor, such as an inactivated cell surface receptor comprising a truncated epithelial growth factor (tEGFR) variant.

In certain embodiments, the promoter is a CAG promoter. In certain embodiments, the CAG promoter comprises the polynucleotide sequence at least 90%, such as at least 90%, 91%, 92%, 93%, 94%, 95%, 96%, 97%, 98% or 100%, identical to SEQ ID NO: 63. Other promoters can also be used, examples of which include, but are not limited to, EFla, UBC, CMV, SV40, PGK1, and human beta actin.

In certain embodiments, the terminator/polyadenylation signal is a SV40 signal. In certain embodiments, the SV40 signal comprises the polynucleotide sequence at least 90%, such as at least 90%, 91%, 92%, 93%, 94%, 95%, 96%, 97%, 98% or 100%, identical to SEQ ID NO: 64. Other terminator sequences can also be used, examples of which include, but are not limited to BGH, hGH, and PGK.

In some embodiment, the vector further comprises a left homology arm and a right homology arm flanking the exogenous polynucleotide.

(3) Nucleic Acids Encoding an HLA Construct

In another general aspect, the invention relates to an isolated nucleic acid encoding an HLA construct useful for an invention according to embodiments of the application. It will be appreciated by those skilled in the art that the coding sequence of an HLA construct can be changed (e.g., replaced, deleted, inserted, etc.) without changing the amino acid sequence of the protein. Accordingly, it will be understood by those skilled in the art that nucleic acid sequences encoding an HLA construct of the application can be altered without changing the amino acid sequences of the proteins.

In certain embodiments, the isolated nucleic acid encodes an HLA construct comprising a signal peptide, such as an HLA-G signal peptide, operably linked to an HLA coding sequence, such as a coding sequence of a mature B2M, and/or a mature HLA-E. In some embodiments, the HLA coding sequence encodes the HLA-G and B2M, which are operably linked by a 4× GGGGS linker, and/or the B2M and HLA-E, which are operably linked by a 3×GGGGS linker. In a particular embodiment, the isolated nucleic acid encoding the HLA construct comprises a polynucleotide sequence at least 90%, such as at least 90%, 91%, 92%, 93%, 94%, 95%, 96%, 97%, 98% or 100%, identical to SEQ ID NO: 67, preferably the polynucleotide sequence of SEQ ID NO: 67. In another embodiment, the isolated nucleic acid encoding the HLA construct comprises a polynucleotide sequence at least 90%, such as at least 90%, 91%, 92%, 93%, 94%, 95%, 96%, 97%, 98% or 100%, identical to SEQ ID NO: 70, preferably the polynucleotide sequence of SEQ ID NO: 70.

In another general aspect, the application provides a vector comprising a polynucleotide sequence encoding a HLA construct useful for an invention according to embodiments of the application. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of a HLA construct in the cell. Conventional cloning techniques or artificial gene synthesis can be used to generate a recombinant expression vector according to embodiments of the application.

In a particular aspect, the application provides vectors for targeted integration of a HLA construct useful for an invention according to embodiments of the application. In certain embodiments, the vector comprises an exogenous polynucleotide having, in the 5′ to 3′ order, (a) a promoter; (b) a polynucleotide sequence encoding an HLA construct; and (c) a terminator/polyadenylation signal.

In certain embodiments, the promoter is a CAG promoter. In certain embodiments, the CAG promoter comprises the polynucleotide sequence at least 90%, such as at least 90%, 91%, 92%, 93%, 94%, 95%, 96%, 97%, 98% or 100%, identical to SEQ ID NO: 63. Other promoters can also be used, examples of which include, but are not limited to, EFla, UBC, CMV, SV40, PGK1, and human beta actin.

In certain embodiments, the terminator/polyadenylation signal is a SV40 signal. In certain embodiments, the SV40 signal comprises the polynucleotide sequence at least 90%, such as at least 90%, 91%, 92%, 93%, 94%, 95%, 96%, 97%, 98% or 100%, identical to SEQ ID NO: 64. Other terminator sequences can also be used, examples of which include, but are not limited to BGH, hGH, and PGK.

In certain embodiments, a polynucleotide sequence encoding a HLA construct comprises a signal peptide, such as a HLA-G signal peptide, a mature B2M, and a mature HLA-E, wherein the HLA-G and B2M are operably linked by a 4×GGGGS linker (SEQ ID NO: 31) and the B2M transgene and HLA-E are operably linked by a 3×GGGGS linker (SEQ ID NO: 25). In particular embodiments, the HLA construct comprises the polynucleotide sequence at least 90%, such as at least 90%, 91%, 92%, 93%, 94%, 95%, 96%, 97%, 98% or 100%, identical to SEQ ID NO: 67, preferably the polynucleotide sequence of SEQ ID NO: 67. In another embodiment, the HLA construct comprises the polynucleotide sequence at least 90%, such as at least 90%, 91%, 92%, 93%, 94%, 95%, 96%, 97%, 98% or 100%, identical to SEQ ID NO: 70, preferably the polynucleotide sequence of SEQ ID NO: 70.

In some embodiment, the vector further comprises a left homology arm and a right homology arm flanking the exogenous polynucleotide.

In certain embodiments, the left homology arm comprises the polynucleotide sequence at least 90%, such as at least 90%, 91%, 92%, 93%, 94%, 95%, 96%, 97%, 98% or 100%, identical to SEQ ID NO: 77. In certain embodiments, the right homology arm comprises the polynucleotide sequence at least 90%, such as at least 90%, 91%, 92%, 93%, 94%, 95%, 96%, 97%, 98% or 100%, identical to SEQ ID NO: 78.

In a particular embodiment, the vector comprises a polynucleotide sequence at least 85%, such as at least 85%, 86%, 87%, 88%, 89%, 90%, 91%, 92%, 93%, 94%, 95%, 96%, 97%, 98% or 100%, identical to SEQ ID NO: 79, preferably the polynucleotide sequence of SEQ ID NO: 79.

(4) Host Cells

In another general aspect, the application provides a host cell comprising a vector of the application and/or an isolated nucleic acid encoding a construct of the application. Any host cell known to those skilled in the art in view of the present disclosure can be used for recombinant expression of exogenous polynucleotides of the application. According to particular embodiments, the recombinant expression vector is transformed into host cells by conventional methods such as chemical transfection, heat shock, or electroporation, where it is stably integrated into the host cell genome such that the recombinant nucleic acid is effectively expressed.

Examples of host cells include, for example, recombinant cells containing a vector or isolated nucleic acid of the application useful for the production of a vector or construct of interest; or an engineered iPSC or derivative cell thereof containing one or more isolated nucleic acids of the application, preferably integrated at one or more chromosomal loci. A host cell of an isolated nucleic acid of the application can also be an immune effector cell, such as a T cell, comprising the one or more isolated nucleic acids of the application. The immune effector cell can be obtained by differentiation of an engineered iPSC of the application. Any suitable method in the art can be used for the differentiation in view of the present disclosure. The immune effector cell can also be obtained transfecting an immune effector cell with one or more isolated nucleic acids of the application.

### IX. Compositions

In another general aspect, the application provides a composition comprising an isolated polynucleotide of the application, a host cell and/or an iPSC or derivative cell thereof of the application.

In certain embodiments, the composition further comprises one or more therapeutic agents selected from the group consisting of a peptide, a cytokine, a checkpoint inhibitor, a mitogen, a growth factor, a small RNA, a dsRNA (double stranded RNA), siRNA, oligonucleotide, mononuclear blood cells, a vector comprising one or more polynucleic acids of interest, an antibody, a chemotherapeutic agent or a radioactive moiety, or an immunomodulatory drug (IMiD).

In certain embodiments, the composition is a pharmaceutical composition comprising an isolated polynucleotide of the application, a host cell and/or an iPSC or derivative cell thereof of the application and a pharmaceutically acceptable carrier. The term “pharmaceutical composition” as used herein means a product comprising an isolated polynucleotide of the application, an isolated polypeptide of the application, a host cell of the application, and/or an iPSC or derivative cell thereof of the application together with a pharmaceutically acceptable carrier. Polynucleotides, polypeptides, host cells, and/or iPSCs or derivative cells thereof of the application and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition described herein or the biological activity of a composition described herein. According to particular embodiments, in view of the present disclosure, any pharmaceutically acceptable carrier suitable for use in a polynucleotide, polypeptide, host cell, and/or iPSC or derivative cell thereof can be used.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carrier can be used in formulating the pharmaceutical compositions of the application.

### X. Methods of Use

In another general aspect, the application provides a method of treating a disease or a condition in a subject in need thereof. The methods comprise administering to the subject in need thereof a therapeutically effective amount of cells of the application and/or a composition of the application. In certain embodiments, the disease or condition is cancer. The cancer can, for example, be a solid or a liquid cancer. The cancer, can, for example, be selected from the group consisting of a lung cancer, a gastric cancer, a colon cancer, a liver cancer, a renal cell carcinoma, a bladder urothelial carcinoma, a metastatic melanoma, a breast cancer, an ovarian cancer, a cervical cancer, a head and neck cancer, a pancreatic cancer, an endometrial cancer, a prostate cancer, a thyroid cancer, a glioma, a glioblastoma, and other solid tumors, and a non-Hodgkin's lymphoma (NHL), Hodgkin's lymphoma/disease (HD), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors. In a preferred embodiment, the cancer is a non-Hodgkin's lymphoma (NHL).

According to embodiments of the application, the composition comprises a therapeutically effective amount of an isolated polynucleotide, an isolated polypeptide, a host cell, and/or an iPSC or derivative cell thereof. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to a cell of the application and/or a pharmaceutical composition of the application a therapeutically effective amount means an amount of the cells and/or the pharmaceutical composition that modulates an immune response in a subject in need thereof.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

In particular embodiments, the cells of the invention are allogeneic to the patient being treated.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

The cells of the application and/or the pharmaceutical compositions of the application can be administered in any convenient manner known to those skilled in the art. For example, the cells of the application can be administered to the subject by aerosol inhalation, injection, ingestion, transfusion, implantation, and/or transplantation. The compositions comprising the cells of the application can be administered transarterially, subcutaneously, intradermaly, intratumorally, intranodally, intramedullary, intramuscularly, inrapleurally, by intravenous (i.v.) injection, or intraperitoneally. In certain embodiments, the cells of the application can be administered with or without lymphodepletion of the subject.

The pharmaceutical compositions comprising cells of the application can be provided in sterile liquid preparations, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

Sterile injectable solutions can be prepared by incorporating cells of the application in a suitable amount of the appropriate solvent with various other ingredients, as desired. Such compositions can include a pharmaceutically acceptable carrier, diluent, or excipient such as sterile water, physiological saline, glucose, dextrose, or the like, that are suitable for use with a cell composition and for administration to a subject, such as a human. Suitable buffers for providing a cell composition are well known in the art. Any vehicle, diluent, or additive used is compatible with preserving the integrity and viability of the cells of the application.

The cells of the application and/or the pharmaceutical compositions of the application can be administered in any physiologically acceptable vehicle. A cell population comprising cells of the application can comprise a purified population of cells. Those skilled in the art can readily determine the cells in a cell population using various well-known methods. The ranges in purity in cell populations comprising genetically modified cells of the application can be from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%. Dosages can be readily adjusted by those skilled in the art, for example, a decrease in purity could require an increase in dosage.

The cells of the application are generally administered as a dose based on cells per kilogram (cells/kg) of body weight of the subject to which the cells and/or pharmaceutical compositions comprising the cells are administered. Generally, the cell doses are in the range of about 10⁴ to about 10¹⁰ cells/kg of body weight, for example, about 10⁵ to about 10⁹, about 10⁵ to about 10⁸, about 10⁵ to about 10⁷, or about 10⁵ to about 10⁶, depending on the mode and location of administration. In general, in the case of systemic administration, a higher dose is used than in regional administration, where the immune cells of the application are administered in the region of a tumor and/or cancer. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 6×10⁸, 7×10⁴ to 1×10⁸, 8×10⁴ to 1×10⁸, 9×10⁴ to 1×10⁸, 1×10⁵ to 1×10⁸, 1×10⁵ to 9×10⁷, 1×10⁵ to 8×10⁷, 1×10⁵ to 7×10⁷, 1×10⁵ to 6×10⁷, 1×10⁵ to 5×10⁷, 1×10⁵ to 4×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 4×10⁷, 2×10⁵ to 3×10⁷, 2×10⁵ to 2×10⁷, 2×10⁵ to 1×10⁷, 2×10⁵ to 9×10⁶, 2×10⁵ to 8×10⁶, 2×10⁵ to 7×10⁶, 2×10⁵ to 6×10⁶, 2×10⁵ to 5×10⁶, 2×10⁵ to 4×10⁶, 2×10⁵ to 4×10⁶, 2×10⁵ to 3×10⁶, 2×10⁵ to 2×10⁶, 2×10⁵ to 1×10⁶, 3×10⁵ to 3×10⁶ cells/kg, and the like. Additionally, the dose can be adjusted to account for whether a single dose is being administered or whether multiple doses are being administered. The precise determination of what would be considered an effective dose can be based on factors individual to each subject.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

The cells of the application and/or the pharmaceutical compositions of the application can be administered in combination with one or more additional therapeutic agents. In certain embodiments the one or more therapeutic agents are selected from the group consisting of a peptide, a cytokine, a checkpoint inhibitor, a mitogen, a growth factor, a small RNA, a dsRNA (double stranded RNA), siRNA, oligonucleotide, mononuclear blood cells, a vector comprising one or more polynucleic acids of interest, an antibody, a chemotherapeutic agent or a radioactive moiety, or an immunomodulatory drug (IMiD). Examples of useful secondary or adjunctive therapeutic agents that can be used with the cells of the invention include, but are not limited to: chemotherapeutic agents including alkylating agents such as thiotepa and cyclophaophamide, alkyl sulfonates such as busulfan, improsulfan and piposulfan; aziridines such as benzodopa, corboquone; ethyleneimines and methylamelamines including altreamine, triethylenemelamine, trietyelenephosphoramide; delta-9-tetrahydocannabinol; a camptothecin, irinotecan, acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB 1-TMI); eleutherobin; pancratistatin; a sarcodictyin; spongistatin; nitrogen mustards such as chlorambucil, chlornaphazine, cholophosphamide, estramustine, ifosfanide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholinodoxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albuminengineered nanoparticle formulation of paclitaxel (ABRAXANET™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; cyclosporine, sirolimus, rapamycin, rapalogs, ibandronate; topoisomerase inhibitor RFS 2000; difluoromethylomithine (DMFO); retinoids such as retinoic acid; CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU, leucovovin; anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY1 17018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other antiandrogens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®); bisphosphonates such as clodronate (for example, BONEFOS® or OST AC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); aptamers, described for example in U.S. Pat. No. 6,344,321, which is herein incorporated by reference in its entirety; anti HGF monoclonal antibodies (e.g., AV299 from Aveo, AMG102, from Amgen); truncated mTOR variants (e.g., CGEN241 from Compugen); protein kinase inhibitors that block mTOR induced pathways (e.g., ARQ197 from Arqule, XL880 from Exelexis, SGX523 from SGX Pharmaceuticals, MP470 from Supergen, PF2341066 from Pfizer); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small molecule inhibitor also known as GW572016); COX-2 inhibitors such as celecoxib (CELEBREX®; 4-(5-(4-methylphenyl)-3-(trifluoromethyl)-1H-pyrazol-1-yl) benzenesulfonamide; and pharmaceutically acceptable salts, acids or derivatives of any of the above.

### EXAMPLES

### Example 1. Generating iPSC-Derived γδ T Cell (γδ iT Cell)

Three methods can be used for creating the iPSCs that are used to make γδ CAR-iT cells. One route uses γδ T cells that are collected from the blood of donors. These T cells possess rearranged γ and δ gene clusters, so when they are reprogrammed to become iPSCs, the resulting TiPSCs (T cell-derived iPSCs) possess the same genetic rearrangements (FIG. 1A). Another method begins with a non-T cell from a donor. The cell type can be any somatic cell, preferably cells used for this process are peripheral blood hematopoietic stem cells (HSCs) that are defined by expression of the surface protein CD34. These PiPSC (peripheral blood CD34 HSC-derived iPSCs), can be converted into a T-PiPSC (TCR-expressing PiPSC) via genetic engineering to knock-in a set of rearranged γδ TCR transgenes (FIG. 1B). A third method uses αβ T cells that are collected from the blood of donors. The αβ T cells can be converted into a T-iPSC (TCR-expressing iPSC) via genetic engineering to knock-in a set of rearranged γδ TCR transgenes (FIG. 1C). The rearranged TCR transgenes for γ and δ chains are delivered as a single polycistronic construct or as two separate constructs: one gamma and one delta.

Generating T Cell-Derived iPSCs (TiPSC)

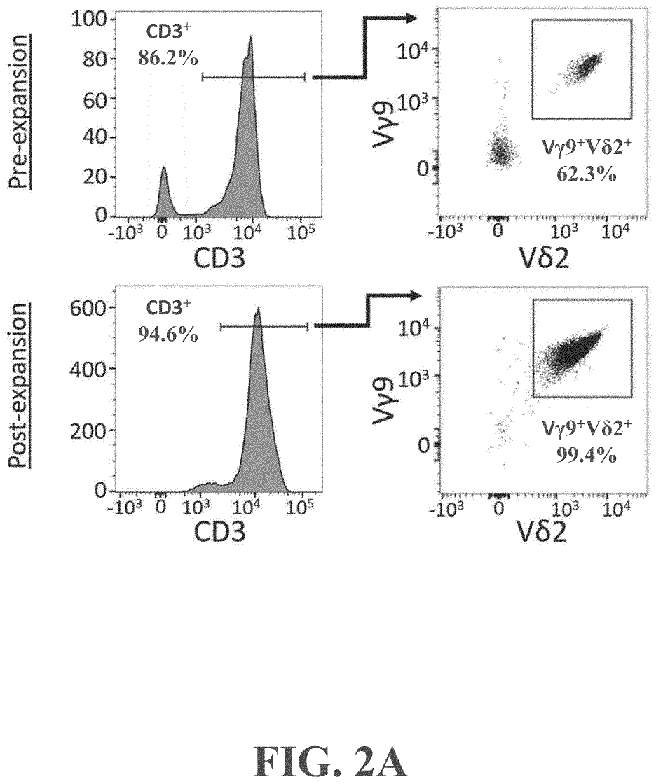

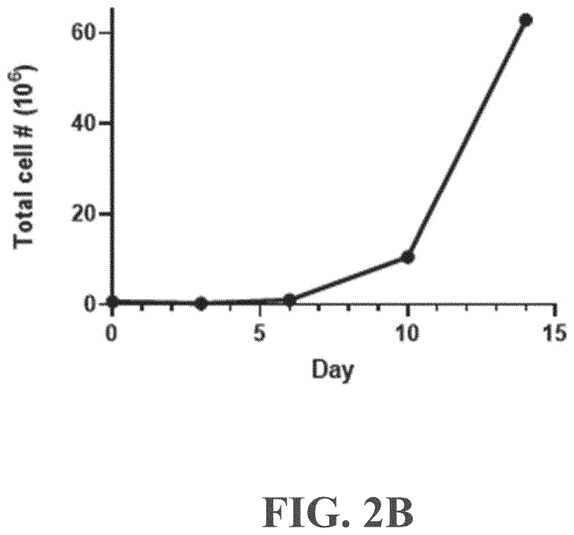

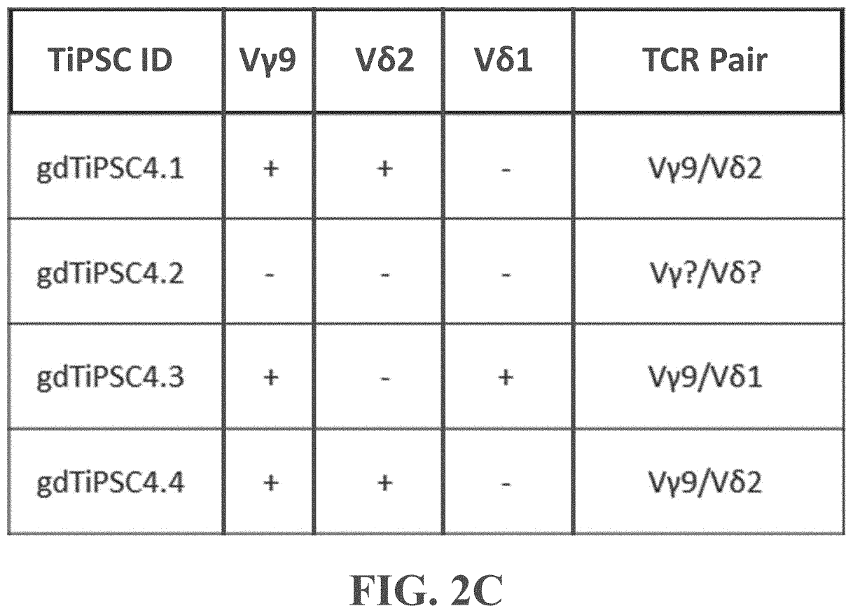

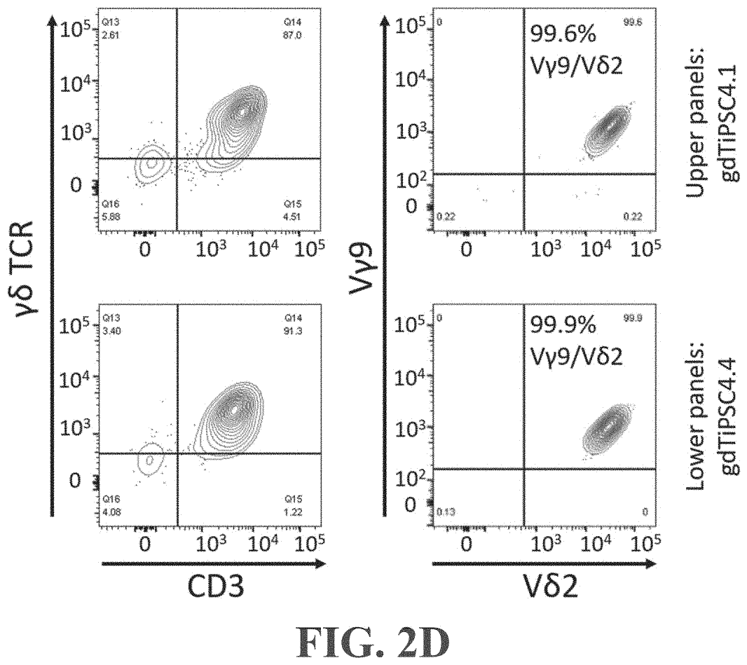

First, peripheral blood mononuclear cells (PBMCs) were collected from healthy donors. From these PBMCs, γδ T cells were enriched by sorting so that 86.2% of live cells were CD3+ T cells, and 62.3% of live CD3+ T cells were Vγ9/Vδ2 T cells by conventional laboratory cell sorting (FIG. 2A). The enriched γδ T cells were cultured in a medium with interleukin-2 (IL-2) and zoledronate for up to two weeks resulting in robust T cell proliferation. The majority of cells at the end of the culture were Vγ9/Vδ2 type γδ T cells. After 14 days, the total cell number increased to ˜63 million cells demonstrating a robust proliferation in response to the bisphosphonate chemical zoledronate and IL-2 (FIG. 2B).

The proliferating γδ T cells were subjected to iPSC reprogramming and several TiPSC lines were successfully derived. The iPSCs were reprogrammed using methods known in the art. Exemplary methods of iPSC reprogramming are described in U.S. Pat. Nos. 8,183,038; 8,268,620; 8,440,461; 9,499,786; 10,865,381; 8,952,801; 8,546,140; 9,644,184; 9,328,332; and 8,765,470, each of which is incorporated by reference in its entirety. Four TiPSC lines were analyzed using a PCR method that detects the presence of a rearranged Vγ9, Vδ2, or Vδ1 TCR gene. Two of the resulting TiPSC lines were Vγ9/Vδ2, one line was Vγ9/Vδ1, and one line did not utilize any of the tested TCR genes (FIG. 2C). The results shown in FIG. 2D demonstrate that the majority of cells were CD3/TCR positive at Day 14 in the two Vγ9/Vδ2 clonal lines.

### Differentiating TiPSC

For generation of hematopoietic progenitor cells (HPCs) from TiPSCs, TiPSCs were cultured in HDM basal medium, composed of 50% Iscove's Modified Dulbecco's Medium and 50% Ham's F12 Nutrient Mixture supplemented with B-27 Supplement, XenoFree, minus Vitamin A (1×), Non-Essential Amino Acids (1×), L-Ascorbic Acid Phosphate Magnesium Salt n-Hydrate (250 uM), Monothioglycerol (100 uM), and Heparin (100 ng/ml). On Day 0, HDM basal medium was supplemented with H1152 (1 uM), CHIR99021 (2 uM), bFGF (50 ng/ml), and VEGF (50 ng/ml). On Day 1, 80% of medium was removed and replaced with HDM basal medium supplemented with CHIR99021 (2 uM), bFGF (50 ng/ml), and VEGF (50 ng/ml). On Days 2, 3, and 4, 80% of medium was removed and replaced with HDM basal medium supplemented with BMP4 (25 ng/ml), bFGF (50 ng/ml), and VEGF (50 ng/ml). On Days 5, 6, 7, 8, 80% of medium was removed and replaced with HDM basal medium supplemented with BMP4 (5 ng/ml), SCF (100 ng/ml), TPO (50 ng/ml), FLT3L (20 ng/ml), and IL-3 (20 ng/ml). HPCs were harvested between days 7-9.

For generation of gamma delta iPSC-derived T (γδ iT) cells from HPCs, HPCs can be purified by positively selecting for cells expressing CD34. Purified and or non-purified HPCs were then cultured in the presence of recombinant Delta-like protein 4 (DLL4; 0.42 ug/cm2) and RetroNectin (0.42 ug/cm2) for 14 days. Cells were fed with TCDM every 24-72 hours. TCDM basal medium used to differentiate HPCs to iT cells was composed of CTS AIM V Medium supplemented with CTS Immune Cell Serum Replacement (10%), Glutamax Supplement (1×), L-Ascorbic Acid Phosphate Magnesium Salt n-Hydrate (250 uM), and Nicotinamide (2 mM). Medium is changed using TCDM basal medium supplemented with SCF (50 ng/ml), FLT3L (50 ng/ml), TPO (50 ng/ml), IL-7 (50 ng/ml). Then the iT cells were further expanded in the presence of mitogenic stimuli including IL-2 (5 ng/ml).

When subjected to iT cell differentiation conditions, the TiPSC yielded iT cells that express a γδ TCR and the T cell lineage marker CD3 (FIG. 2D). The TiPSC approach was also used with a γδ TiPSC line that was reprogrammed from healthy donor T cells without any attempt to enrich for or expand bisphosphonate-responsive γδ T cells.

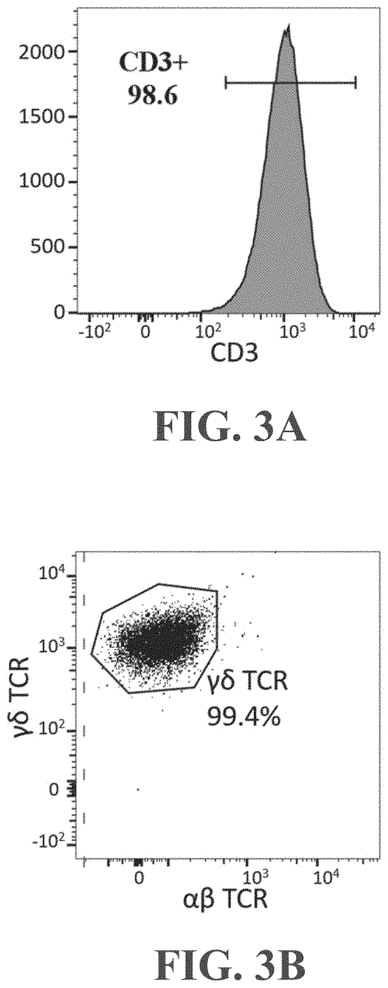

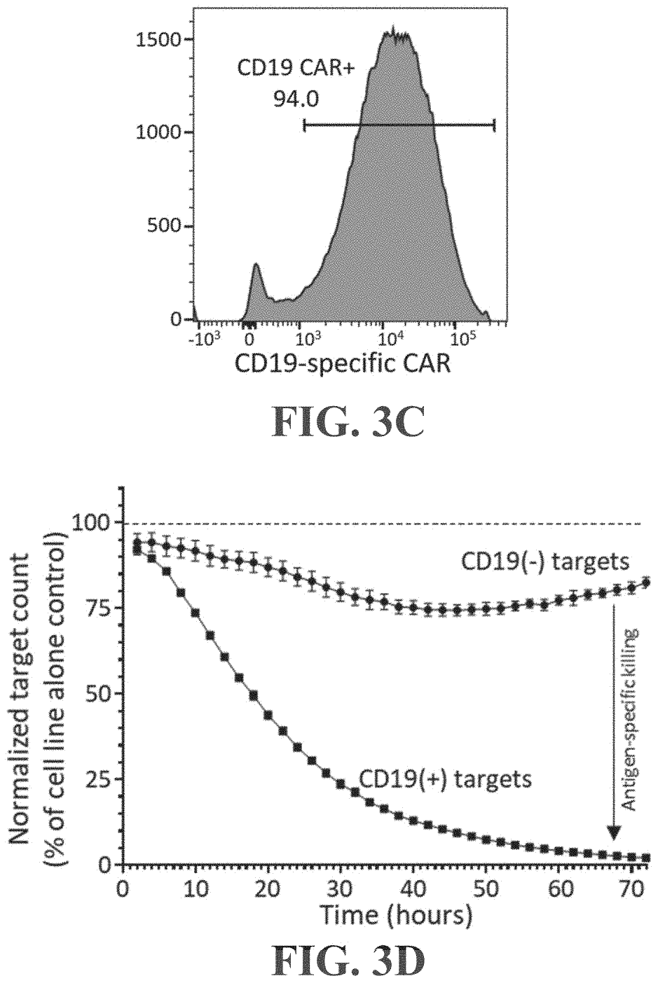

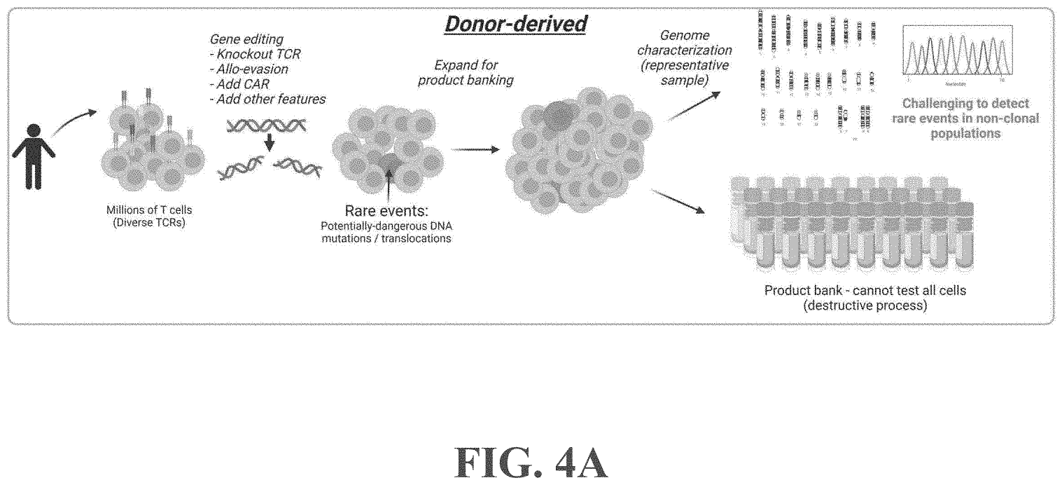

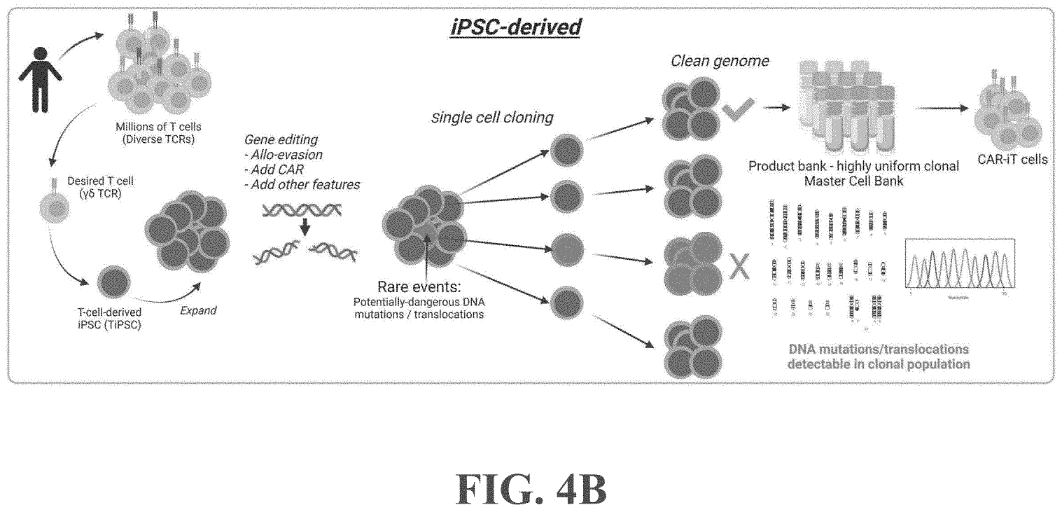

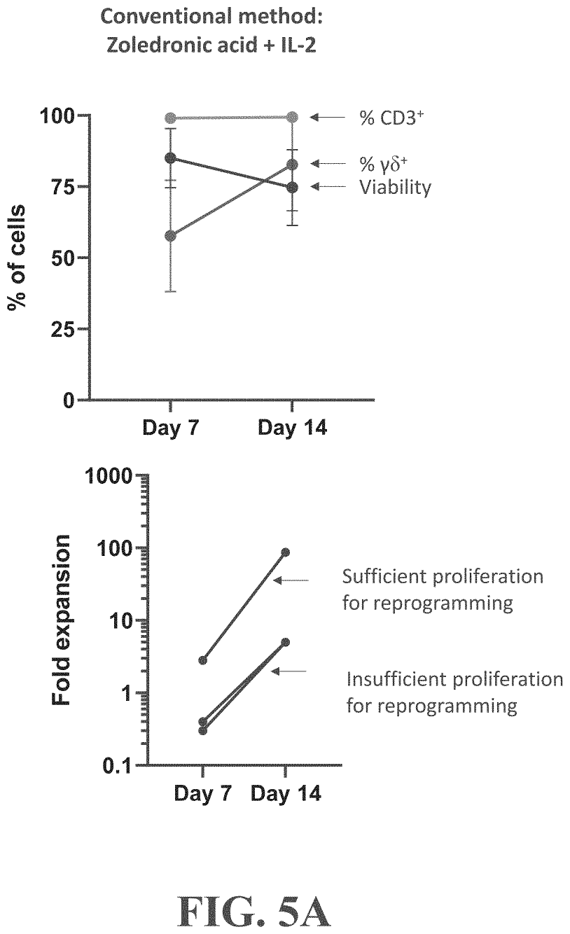

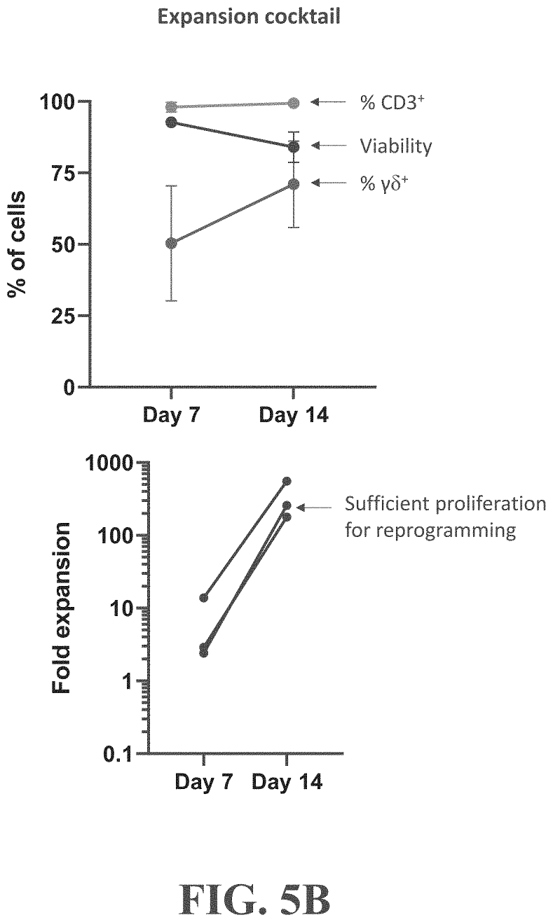

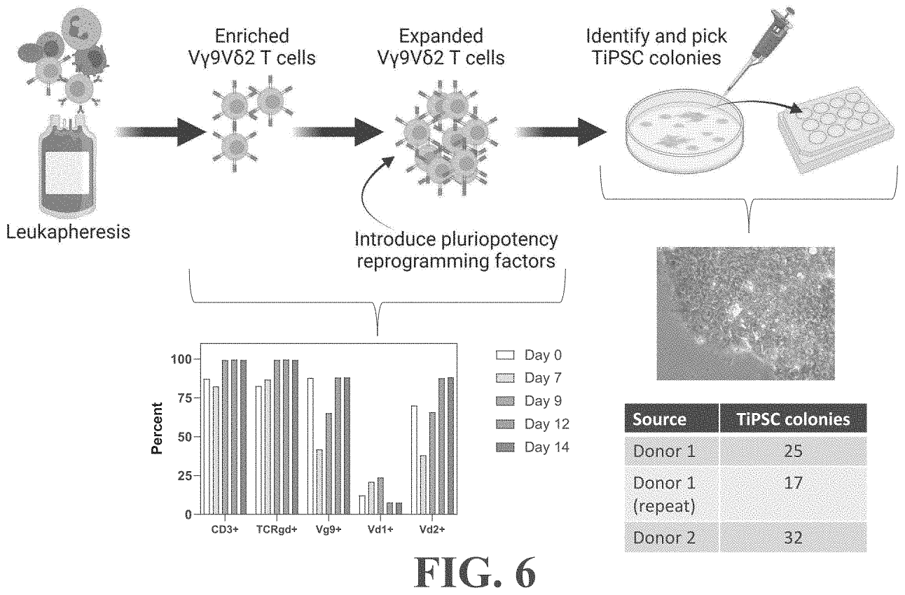

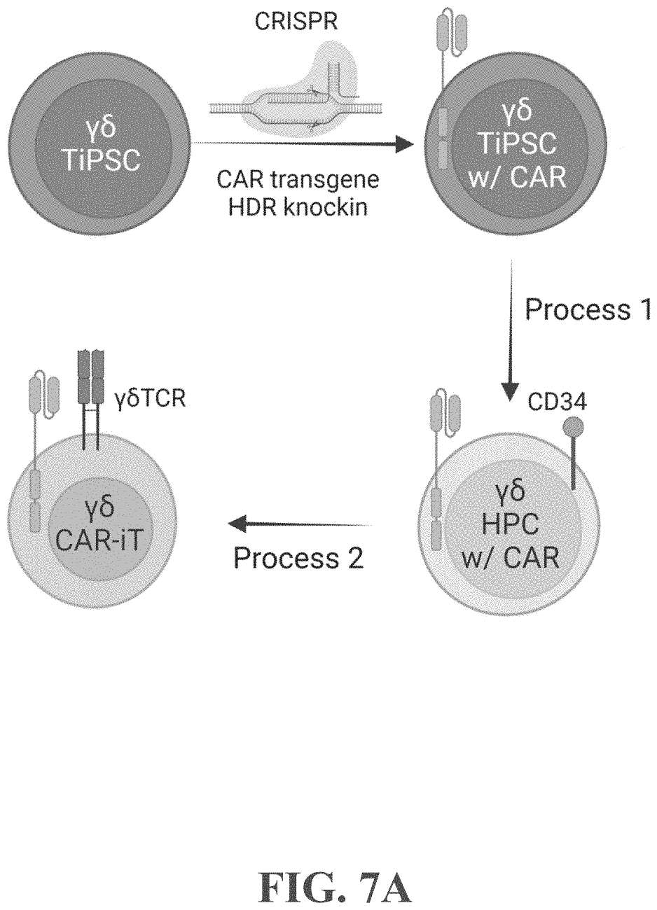

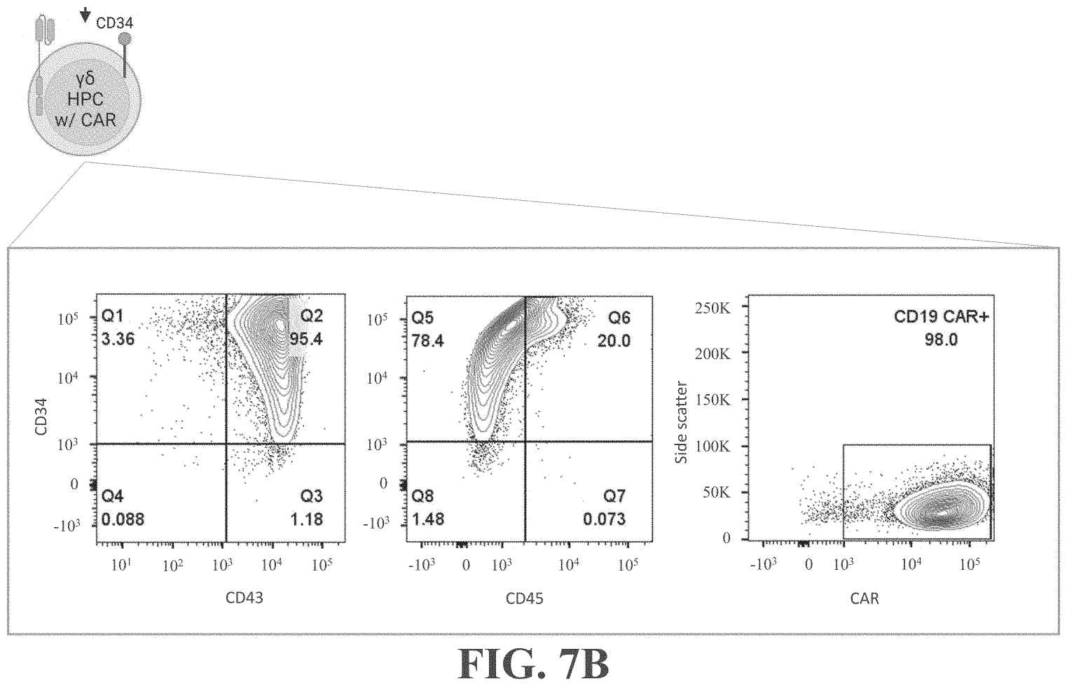

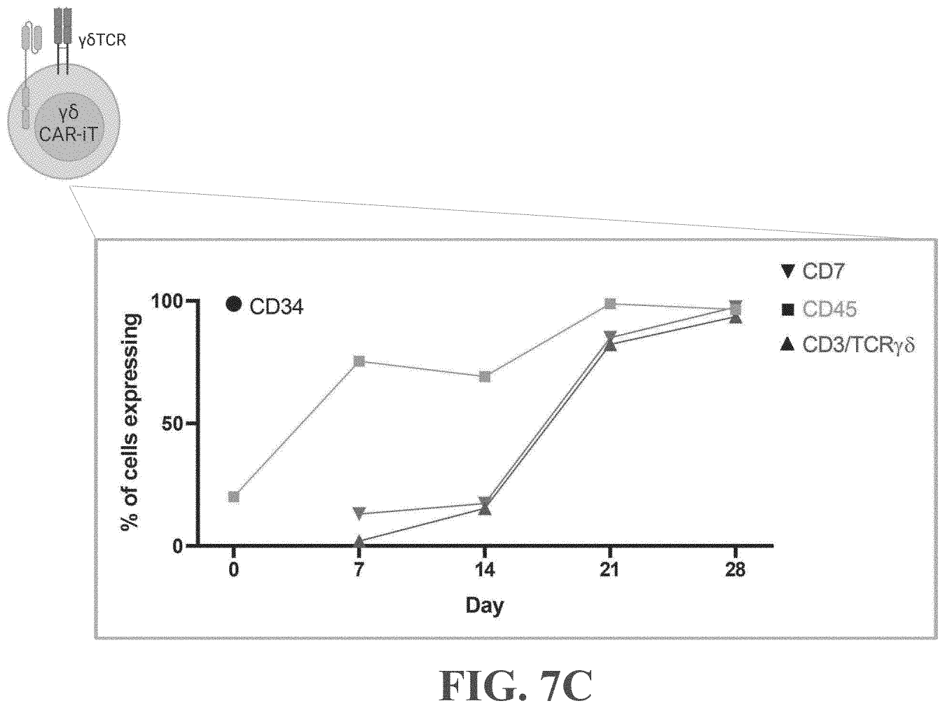

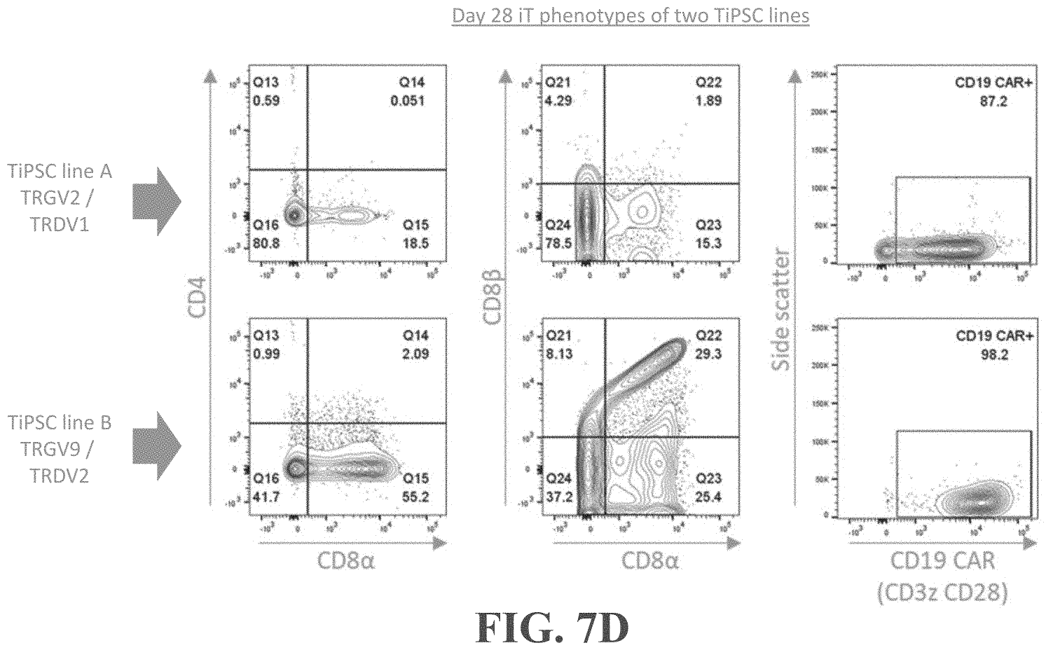

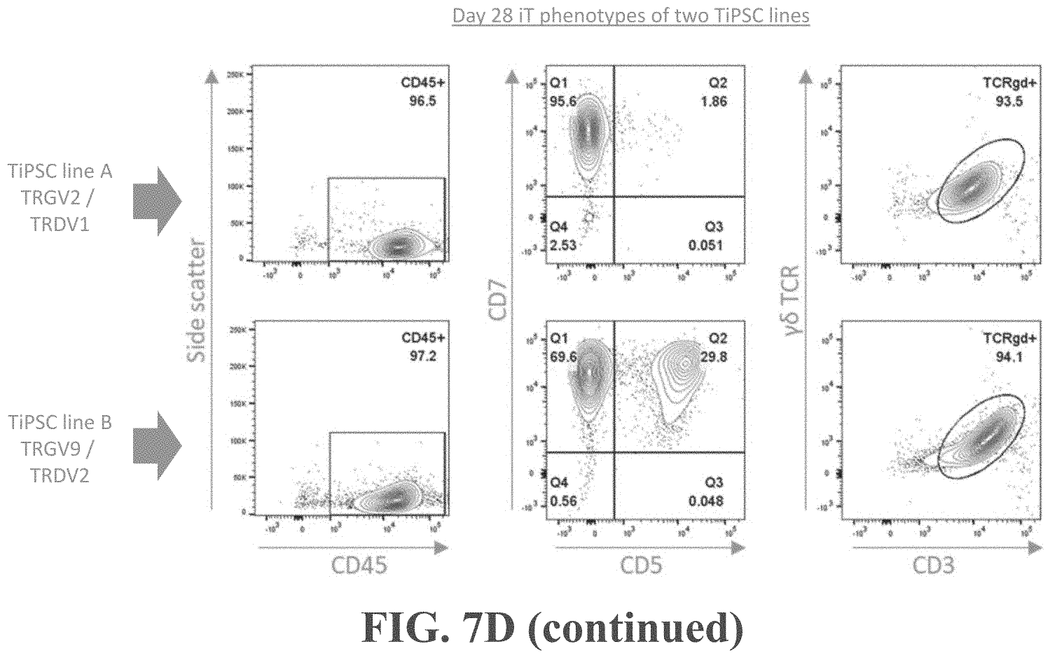

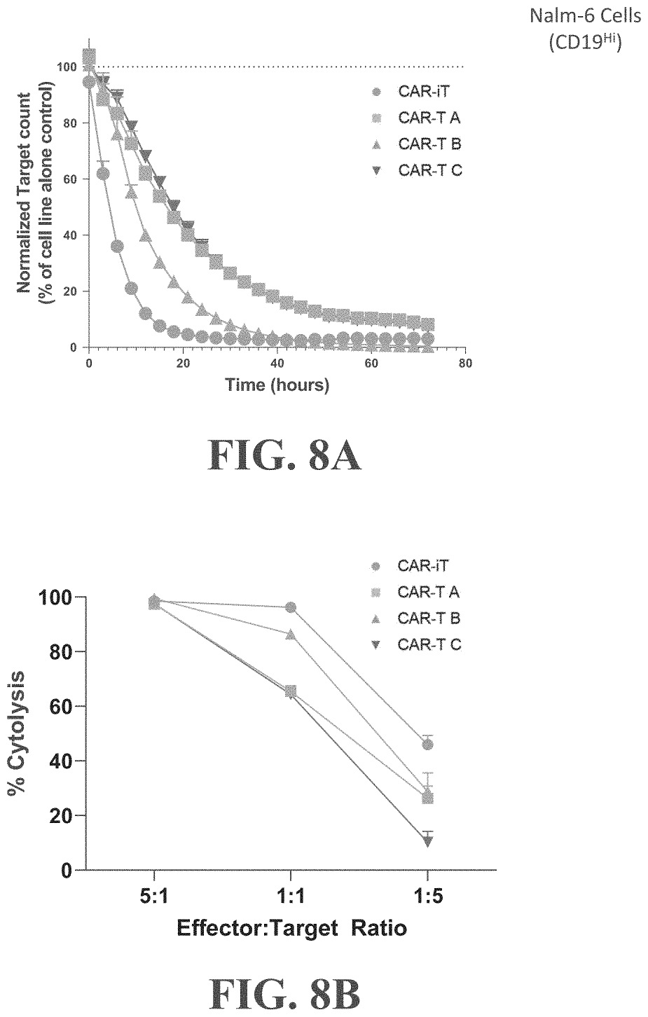

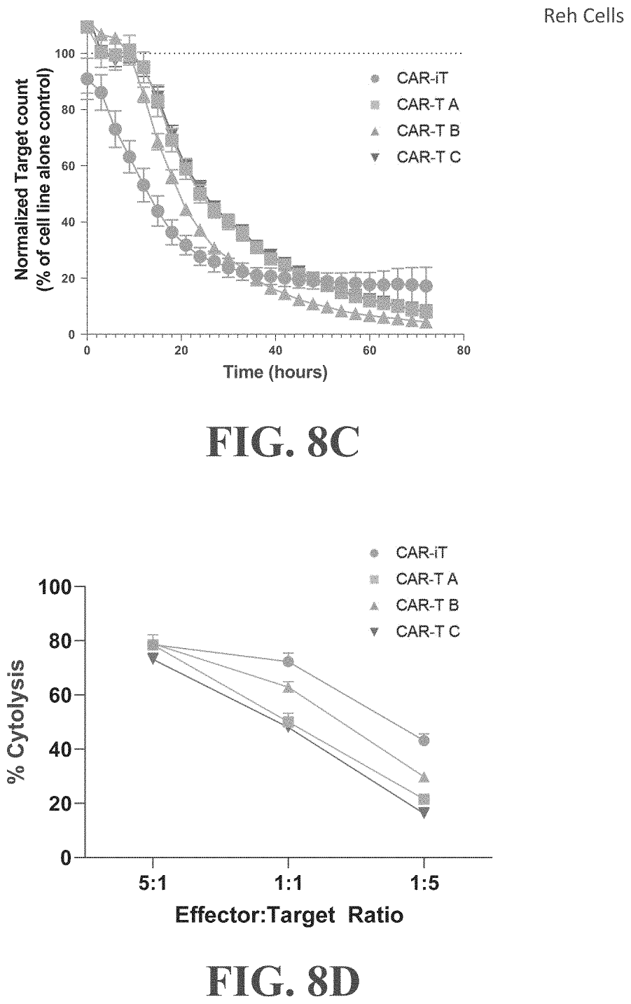

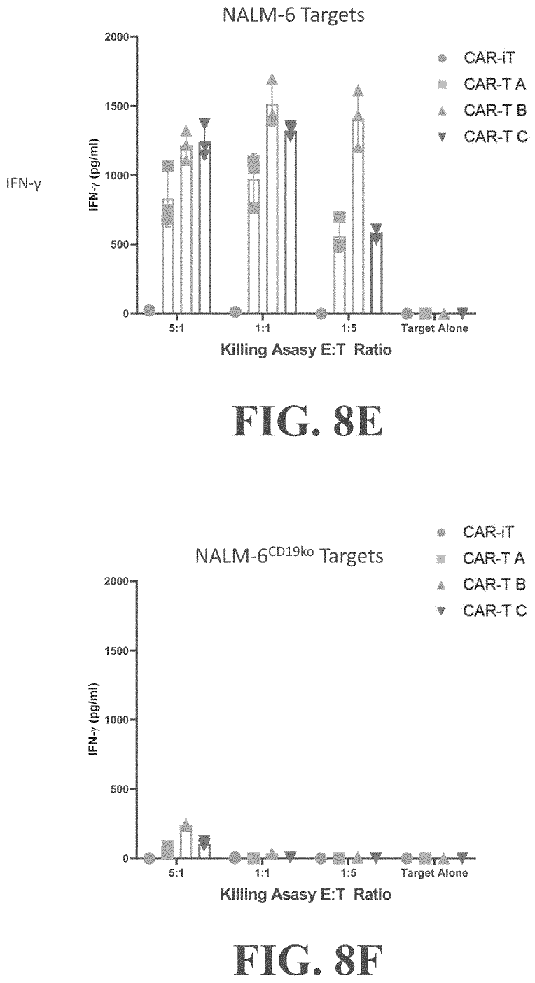

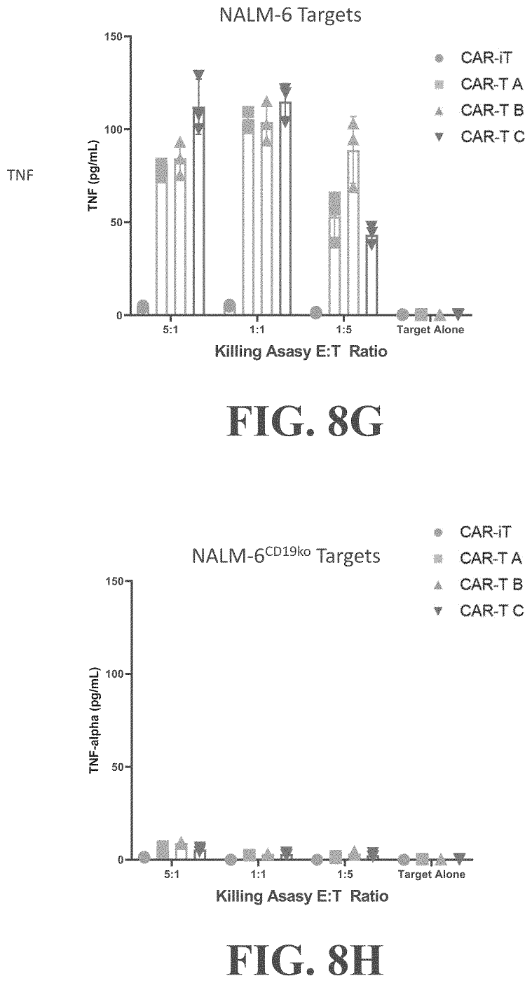

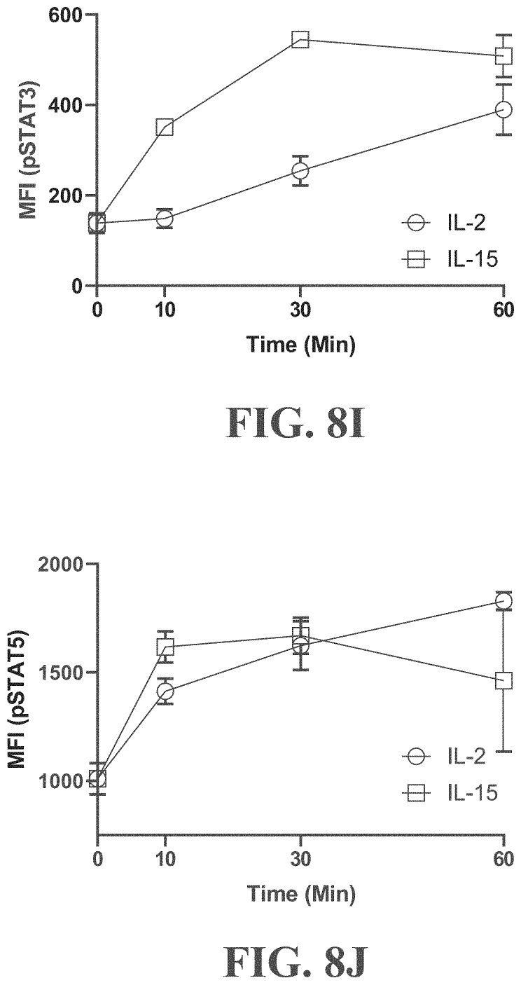

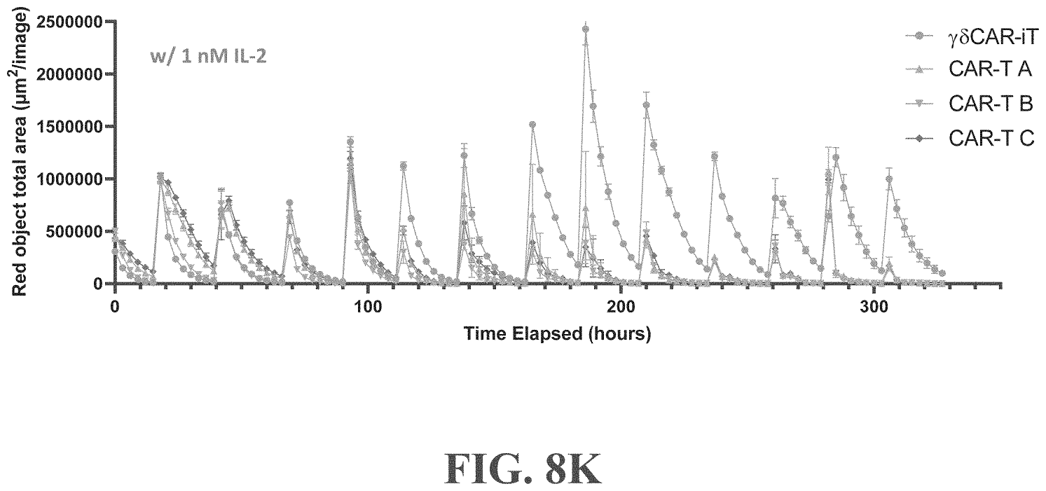

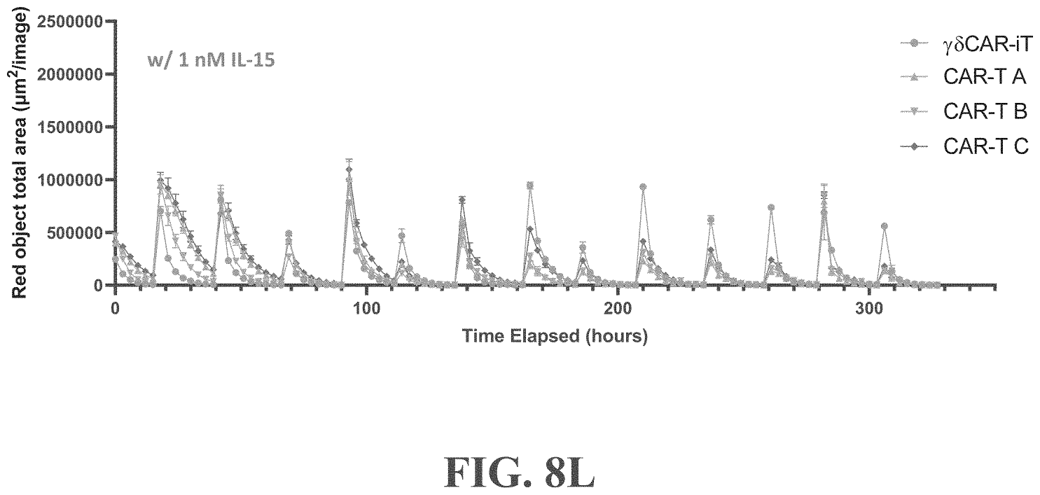

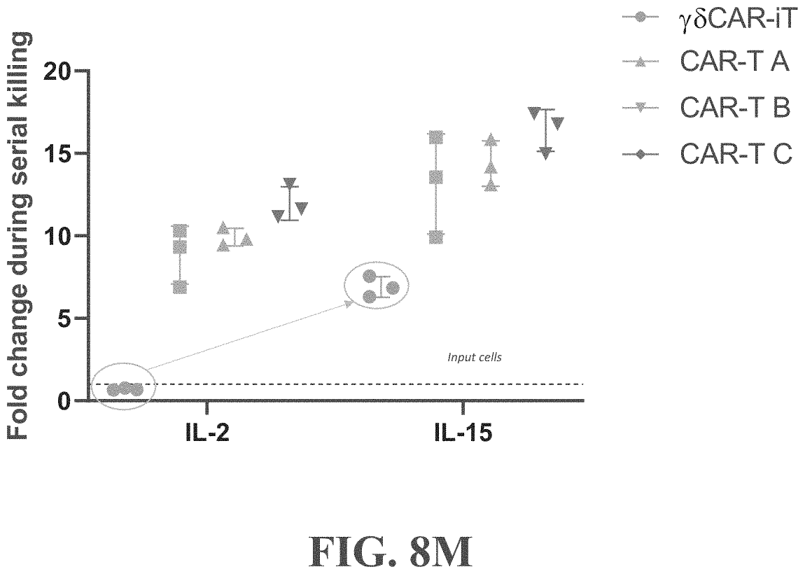

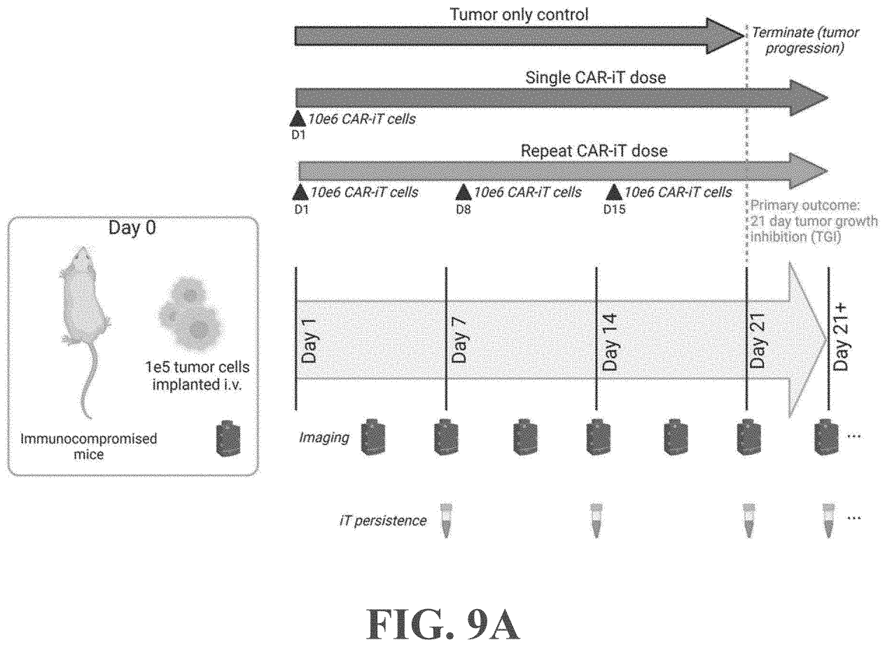

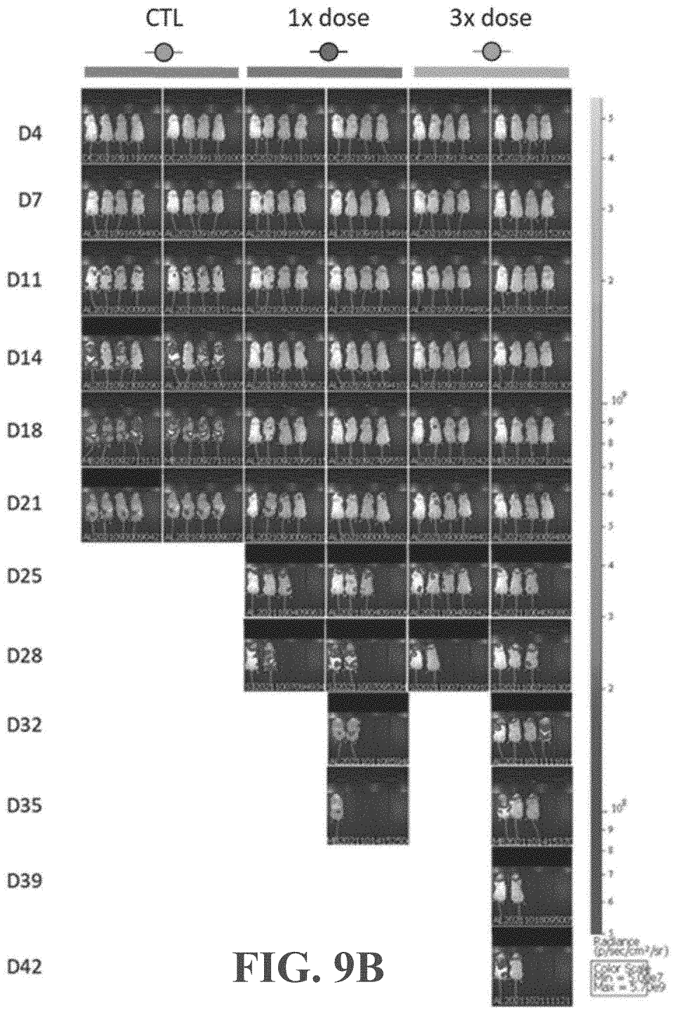

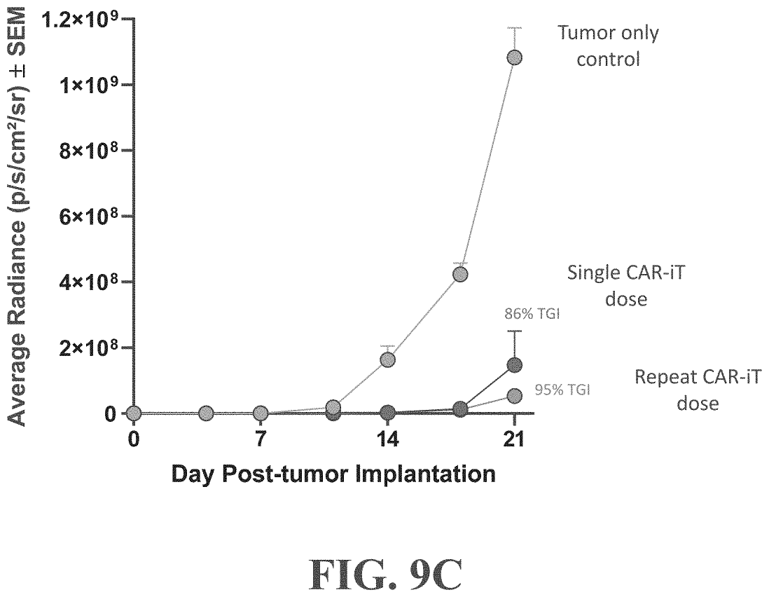

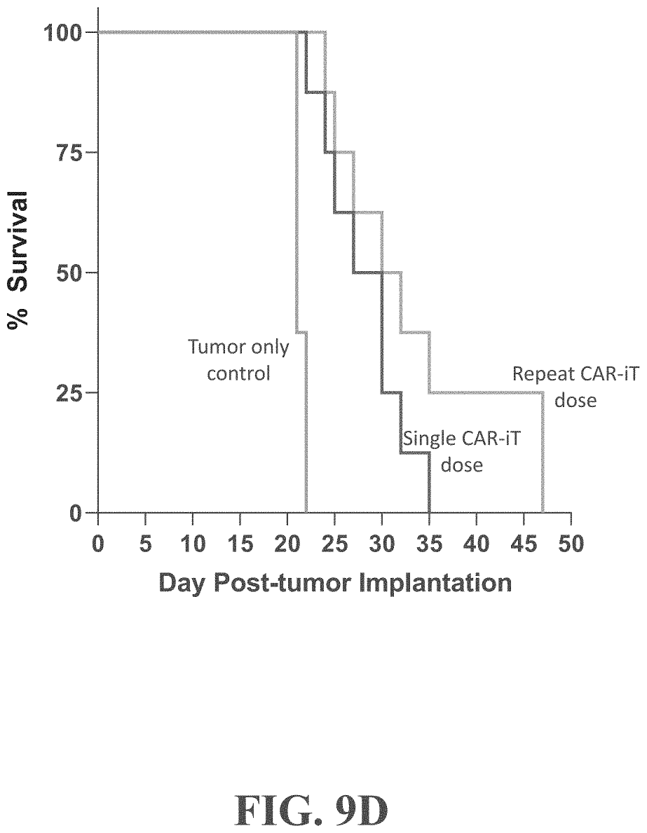

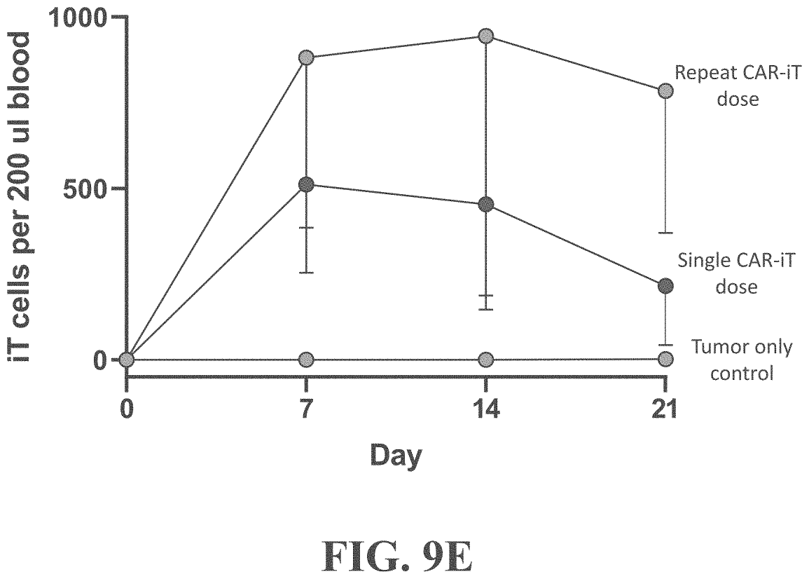

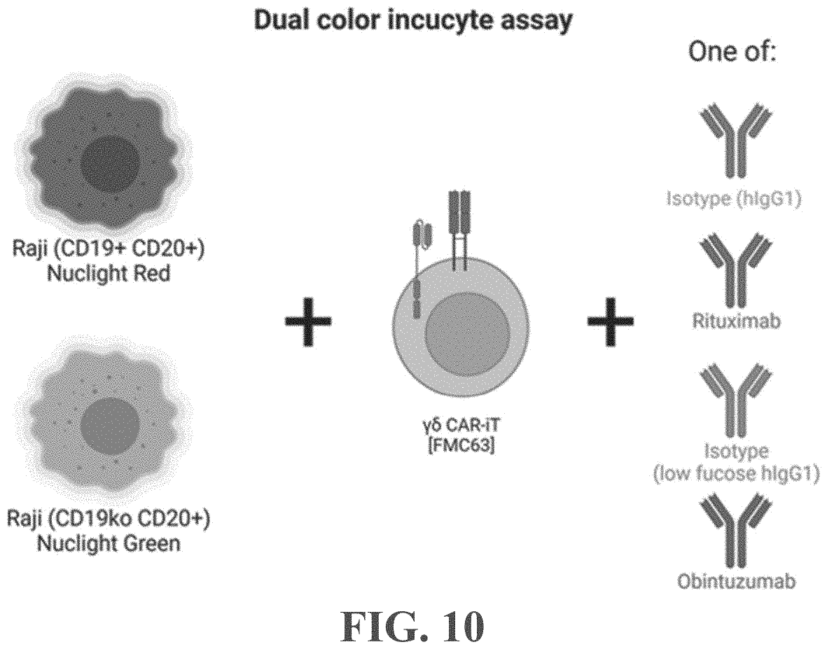

The resulting cells were uniformly CD3-positive and γδ TCR-positive/αβ TCR-negative (FIGS. 3A-B). The TiPSC line was also engineered to express a CD19 CAR under an exogenous constitutive promoter leading to CD19 CAR expression in the iT cells (FIG. 3C). When CAR-iT cells were used in an IncuCyte-based killing assay at an effector to target ratio of 1:10 (1 iT cell per 10 Reh B cell leukemia cells), the iT cells potently killed CD19+ Reh cells, but did not kill Reh cells where CD19 had been deleted by gene editing (FIG. 3D).

### Example 2. Identifying Functional γδ TCR Pairs

In order to deliver a TCR transgene to a PiPSC, it is important to select a functional TCR pair. The γ and δ chains must not only be expressed, but they must be able to assemble together as a functional heterodimer. Because thymic selection tests TCR pairs for function (developing T cells lacking a functional TCR heterodimer are eliminated in the thymus), mature peripheral blood T cells will, as a rule, possess a functional TCR heterodimer. To identify trusted TCRs, single T cells were sorted and RNA sequencing was utilized to identify the specific rearranged polynucleotide sequences that work together as a functional γδ TCR heterodimer. Vγ9/Vδ2 γδ T cells that were expanded using bisphosphonates were purified (FIG. 2A). These cells were then sorted as single cells and the CDR3 region of each rearranged TCR gene was determined by sequencing. Table 3 is a listing of 13 TCR pairs that were identified. Because the T cells that were used for Table 3 were expanded using bisphosphonates (zoledronate), the TCRs listed are putatively bisphosphonate-responsive. These CDR3 regions, the imputed V gene germline sequence, and the germline γ and δ constant regions (TRGC1, TRGC2, or TRDC) are used to assemble a synthetic TCR construct that recapitulates the original TCR pairing.

TABLE 3
TCR γδ pairs from single γδ T cells

| Pair | Gamma locus | | | Delta locus | | | |
|---|---|---|---|---|---|---|---|
| ID | V | J | CDR3 | V | D | J | CDR3 |
| A02 | TRGV9 | TRGJP | ALWEVQELGKKIKV | TRDV2 | TRDD3 | TRDJ1 | ACDTVGVGVVYTDKLI |
| | | | (SEQ ID NO: 109) | | | | (SEQ ID NO: 110) |
| B11 | TRGV9 | TRGJP | ALWREQELGKKIKV | TRDV2 | — | TRDJ1 | ACDTVERSTRTYTDKLI |
| | | | (SEQ ID NO: 111) | | | | (SEQ ID NO: 112) |
| C02 | TRGV9 | TRGJP | ALWVVLGKKIKV | TRDV2 | TRDD3 | TRDJ1 | ACDTVSRAGTGGHTTDKLI |
| | | | (SEQ ID NO: 113) | | | | (SEQ ID NO: 114) |
| D06 | TRGV9 | TRGJP | ALWEVGELGKKIKV | TRDV2 | TRDD3 | TRDJ1 | ACDTVKRGTHALI |
| | | | (SEQ ID NO: 115) | | | | (SEQ ID NO: 116) |
| D09 | TRGV9 | TRGJP | ALWEEELGKKIKV | TRDV2 | TRDD3 | TRDJ1 | ACDILGDTDKLI |
| | | | (SEQ ID NO: 117) | | | | (SEQ ID NO: 118) |
| D11 | TRGV9 | TRGJP | ALWEVRELGKKIKV | TRDV2 | TRDD3 | TRDJ1 | ACDSVLGDLYTDKLI |
| | | | (SEQ ID NO: 119) | | | | (SEQ ID NO: 120) |
| E09 | TRGV9 | TRGJP | ALWEVGELGKKIKV | TRDV2 | TRDD3 | TRDJ1 | ACDTVKRGTHALI |
| | | | (SE QID NO: 121) | | | | (SEQ ID NO: 122) |
| E10 | TRGV9 | TRGJP | ALWEVFQELGKKIKV | TRDV2 | TRDD3 | TRDJ1 | ACDTLGMPGDTDTDKLI |
| | | | (SEQ ID NO: 123) | | | | (SEQ ID NO: 124) |
| E12 | TRGV9 | TRGJP | ALWEVFGLGKKIKV | TRDV2 | TRDD3 | TRDJ1 | ACDDLRGRDTDKLI |
| | | | (SEQ ID NO: 125) | | | | (SEQ ID NO: 126) |
| G11 | TRGV9 | TRGJP | ALWEVFQELGKKIKV | TRDV2 | TRDD3 | TRDJ1 | ACDTLGMPGDTDTDKLI |
| | | | (SEQ ID NO: 123) | | | | (SEQ ID NO: 124) |
| H09 | TRGV9 | TRGJP | ALWEVLPKLGKKIKV | TRDV2 | TRDD3 | TRDJ1 | ACDMLLGDTDTDKLI |
| | | | (SEQ ID NO: 127) | | | | (SEQ ID NO: 128) |
| H10 | TRGV9 | TRGJP | ALWDLGELGKKIKV | TRDV2 | TRDD3 | TRDJ1 | ACDSLGDSGYTDKLI |
| | | | (SEQ ID NO: 129) | | | | (SEQ ID NO: 130) |
| H12 | TRGV9 | TRGJP | ALWELRKKL | TRDV2 | TRDD3 | TRDJ1 | ACDFVLGAMSDKLI |
| | | | (SEQ ID NO: 131) | | | | (SEQ ID NO: 132) |
| | TRGV9 | TRGJP | ALWEVQELGKKIKVF | TRDV2 | TRDD3 | TRDJ1 | ACDTVGDTDKLI |
| | | | (SEQ ID NO: 152) | | | | (SEQ ID NO: 153) |
| | TRGV9 | TRGJP | ALWEGTPGELGKKIKV | TRDV2 | TRDD3 | TRDJ1 | ACDRLGDTRTKLIF |
| | | | (SEQ ID NO: 154) | | | | (SEQ ID NO: 155) |

### Example 3. Generating iPSC-Derived γδ T Cell (γδ iT Cell) with Recombinant TCRs

Generating Peripheral Blood CD34 HSC-Derived iPSCs (PiPSC)

First, peripheral blood mononuclear cells (PBMCs) are collected from healthy donors. Then, hematopoietic stem cells (HSCs) are isolated that are defined by expression of the surface protein CD34.

The proliferating HSCs are subjected to iPSC reprogramming. Methods of iPSC reprogramming are well known in the art and any can used in the described method in view of the present disclosure. Exemplary methods of iPSC reprogramming are described in U.S. Pat. Nos. 8,183,038; 8,268,620; 8,440,461; 9,499,786; 10,865,381; 8,952,801; 8,546,140; 9,644,184; 9,328,332; and 8,765,470, each of which is incorporated by reference in its entirety. CD34+ HSC-derived iPSC (PiPSC) are genetically engineered to express a recombinant rearranged γδ TCR. The rearranged TCR transgenes for γ and δ chains are delivered as a single polycistronic construct or as two separate constructs: one gamma and one delta. The rearranged γδ TCR can be any one of those listed in Table 3. The rearranged γδ TCR can comprises a γ TCR chain having the amino acid sequence of SEQ ID NO: 133, 135, or 150, and a δ TCR chain having the amino acid sequence of SEQ ID NO: 134, 136, or 151. The recombinant rearranged γδ TCR is integrated at a loci on the chromosome of the cell selected from the group TRAC, TRBC1, TRBC2. Additional gene editing can be done at the PiPSC stage, including, but not limited to, gene knock-in and gene knock-out.

### Differentiating PiPSCs

For generation of HPCs from PiPSCs, PiPSCs are cultured in HDM basal medium, composed of 50% Iscove's Modified Dulbecco's Medium and 50% Ham's F12 Nutrient Mixture supplemented with B-27 Supplement, XenoFree, minus Vitamin A (1×), Non-Essential Amino Acids (1×), L-Ascorbic Acid Phosphate Magnesium Salt n-Hydrate (250 uM), Monothioglycerol (100 uM), and Heparin (100 ng/ml). On Day 0, HDM basal medium is supplemented with H1152 (1 uM), CHIR99021 (2 uM), bFGF (50 ng/ml), and VEGF (50 ng/ml). On Day 1, 80% of medium is removed and replaced with HDM basal medium supplemented with CHIR99021 (2 uM), bFGF (50 ng/ml), and VEGF (50 ng/ml). On Days 2, 3, and 4, 80% of medium is removed and replaced with HDM basal medium supplemented with BMP4 (25 ng/ml), bFGF (50 ng/ml), and VEGF (50 ng/ml). On Days 5, 6, 7, 8, 80% of medium is removed and replaced with HDM basal medium supplemented with BMP4 (5 ng/ml), SCF (100 ng/ml), TPO (50 ng/ml), FLT3L (20 ng/ml), and IL-3 (20 ng/ml). HPCs are harvested between days 7-9 depending on the original cell type used to derive the iPSC. HPC are defined as CD34+, CD43+, +/−CD45 on the cell surface.

For generation of γδ iPSC-derived T cells from HPCs, HPCs can be purified by positively selecting for cells expressing CD34. Purified and or non-purified HPCS are then cultured in the presence of recombinant Delta-like protein 4 (SEQ ID NOs: 139-141) (DLL4; 0.42 ug/cm2) and RetroNectin (0.42 ug/cm2) for up to 35 days. In some embodiments, variants of DLL4 are included (SEQ ID NOs: 142-144) as well as other fibronectin proteins in combination with or in replacement of RetroNectin. In certain embodiments, recombinant Jagged 2 (JAG2) is used. In further embodiments, the recombinant proteins can be immobilized using polydopamine in the presence or absence of Protein G coating to facilitate proper orientation of the recombinant proteins. Cells are fed with TCDM every 24-72 hours and collected for passaging in the presence of aforementioned protein combinations, typically every 7 days. TCDM basal medium used to differentiate HPCs to iT cells is composed of CTS AIM V Medium supplemented with CTS Immune Cell Serum Replacement (10%), Glutamax Supplement (1×), L-Ascorbic Acid Phosphate Magnesium Salt n-Hydrate (250 uM), and Nicotinamide (2 mM). Medium is changed using TCDM basal medium supplemented with SCF (50 ng/ml), FLT3L (50 ng/ml), TPO (50 ng/ml), IL-7 (50 ng/ml). The medium can further comprise CHIR99021 (2 uM), and/or IL-2 (5 ng/ml). γδ iT cells are defined as CD3+, γδ TCR+, CD45RA+/−, CD7+, CD5+/−, CD56+/−, CD8α+/−. iT cells are positive for at least one of the markers CD62L, CD27, CD28, or CCR7.

### Example 4. iPSC-Derived CAR-γδ T Cells Perform Antibody-Dependent Cellular Cytotoxicity (ADCC)

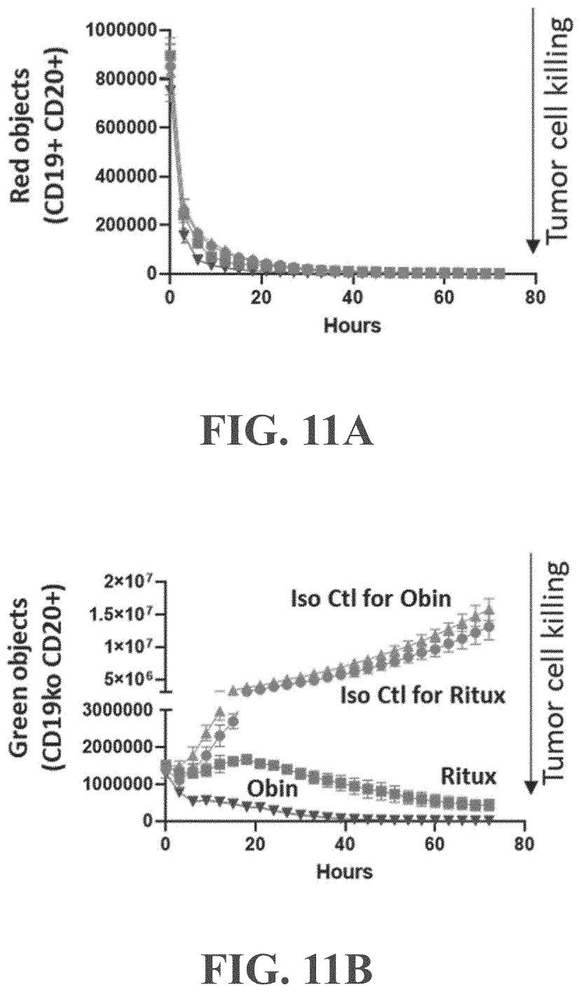

Two tumor cell lines were prepared for an ADCC assay. For the ADCC assay, both cell types were mixed at an equal ratio and then incubated in an Incucyte cell killing assay where iPSC-derived CAR-γδ T cells (iT cells) were the effectors. These CAR-γδ iT cells express a CD19-specific CAR (FMC63 anti-CD19 scFv) and were expected to eliminate CD19+ tumor cells. Because γδ T cells are known to sometimes express the Fc-gamma receptor CD16, we evaluated the ability of CAR-γδ iT cells to kill targets via ADCC. CAR-γδ T cell performed ADCC of the Raji lymphoblastic B cell line, which normally expresses B cell antigens CD19 and CD20 and was engineered with a transgene encoding a red fluorescent protein (FIG. 11A). Two different anti-CD20 antibodies (Rituximab and Obinutuzumab) were tested in comparison to their relevant isotype controls. Red tumor cells (CD19+) were eliminated by CAR-γδ iT cells in all conditions. CAR-γδ T cell performing ADCC of Raji cells that were modified using CRISPR gene editing to knockout (KO) the gene encoding CD19, and then engineered with a transgene encoding a green fluorescent protein (FIG. 11B). Two different anti-CD20 antibodies (Rituximab and Obinutuzumab) were tested in comparison to their relevant isotype controls. Green CD19KO tumor cells were spared from killing in the presence of isotype control antibodies. The presence of Rituximab or Obinutuzumab enabled CAR-γδ iT cells to kill CD19KO cells via ADCC.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. An induced pluripotent stem cell (iPSC) that comprises:
(i) one or more polynucleotides encoding a recombinant rearranged γδ T cell receptor (TCR); and
(ii) a polynucleotide encoding a chimeric antigen receptor (CAR);
wherein the recombinant rearranged γδ TCR is not specific to the binding target of the CAR and supports differentiation of the iPSC to a T cell.

2. The iPSC according to claim 1, wherein the recombinant rearranged γδ TCR enables expansion of the T cell differentiated from the iPSC after mitogenic stimulation.

3. The iPSC according to claim 1, wherein the one or more polynucleotides encoding the recombinant rearranged γδ TCR comprise a γ TCR variable gene selected from a group consisting of TRGV2-5, TRGV8 and TRGV9 genes; a γ TCR joining gene selected from the group consisting of TRGJ1, TRGJ2, TRGJP, TRGJP1 and TRGJP2 genes; and a γ TCR constant genes selected from the group consisting of TRGC1 and TRGC2 genes.

4. The iPSC according to claim 1, wherein the one or more polynucleotides encoding the recombinant rearranged γδ TCR comprise a δ TCR variable gene selected from the group consisting of TRDV1-3 genes; a δ TCR diversity genes selected from the group consisting of TRDD1, TRDD2 and TRDD3 genes; a δ TCR joining genes selected from the group consisting of TRDJ1, TRDJ2, TRDJ3 and TRDJ4; and a δ TCR constant gene TRDC.

5. The iPSC according to claim 1, wherein the one or more polynucleotides encoding the recombinant rearranged γδ TCR comprise a γ TCR variable gene TRGV9 and a δ TCR variable gene TRDV2.

6. The iPSC according to claim 1, wherein the recombinant rearranged γδ TCR is activated by one or more phospho-antigens selected from isopentenyl pyrophosphate (IPP), dimethylallyl diphosphate (DMAPP), and (E)-4-hydroxy-3-methyl-but-2-enylpyrophosphate (HMBPP), or chemically similar molecules, wherein the phospho-antigens are naturally-occurring in cells as products of metabolic processes or the phospho-antigens are caused to accumulate in cells at higher levels due to treatment with bisphosphonate chemicals, wherein the activity of the phospho-antigens is through direct interaction with the γδ TCR or the activity of phospho-antigens is through interactions with butyrophilin (BTN) proteins BTN2A1, BTN3A1, BTN3A2, or BTN3A3.

7. The iPSC according to claim 1, wherein the recombinant rearranged γδ TCR is not activated by phospho-antigens.

8. The iPSC according to claim 1, wherein the iPSC is reprogrammed from peripheral blood mononuclear cells (PBMCs), preferably CD34+ hematopoietic stem cells (HSCs), αβ T cells or γδ T cells.

9. The iPSC according to claim 8, wherein the iPSC is prepared by expanding the PBMCs in the presence of an amino-bisphosphonate and interleukin 2 (IL2) prior to incorporating reprogramming transcription factors into the PBMC to generate the iPSC.

10. The iPSC according to claim 9, wherein the amino-bisphosphonate is zoledronic acid or salts thereof.

11. A T cell derived from the iPSC according to claim 1.

12. An induced pluripotent stem cell (iPSC) or a T cell derived therefrom comprising: one or more polynucleotides encoding a rearranged γδ T cell receptor (TCR) and an exogenous polynucleotide encoding a chimeric antigen receptor (CAR); and one or more of:
a. an exogenous polynucleotide encoding an artificial cell death polypeptide;
b. a deletion or reduced expression of one or more of B2M, TAP 1, TAP 2, Tapasin, RFXANK, CIITA, RFX5 and RFXAP genes;
c. a deletion or reduced expression of RAG1 and RAG2 genes;
d. an exogenous polynucleotide encoding a non-naturally occurring variant of FcγRIII (CD16);
e. an exogenous polynucleotide encoding interleukin 15 (IL-15) and/or IL-15 receptor or a variant or truncation thereof;
f. an exogeneous polynucleotide encoding a constitutively active interleukin 7 (IL-7) receptor or variant thereof;
g. an exogenous polynucleotide encoding interleukin 12 (IL-12) or interleukin 21 (IL-21) or a variant thereof;
h. an exogenous polynucleotide encoding human leukocyte antigen E (HLA-E) and/or human leukocyte antigen G (HLA-G);
i. an exogenous polynucleotide encoding leukocyte surface antigen cluster of differentiation CD47 (CD47) and/or CD24; and
j. an exogenous polynucleotide encoding one or more imaging or reporter proteins, such as PSMA or HSV-tk.

13. The iPSC or T cell according to claim 12, wherein the rearranged γδ TCR enables increased expansion of the T cell after mitogenic stimulation than a T cell without the rearranged TCR.

14. The iPSC or T cell according to claim 13, wherein the iPSC is reprogrammed from peripheral blood mononuclear cells (PBMCs), preferably CD34+ hematopoietic stem cells (HSCs), αβ T cells or γδ T cells.

15. The iPSC or T cell according to claim 12, wherein the rearranged γδ TCR is activated by one or more phospho-antigens selected from isopentenyl pyrophosphate (IPP), dimethylallyl diphosphate (DMAPP), and (E)-4-hydroxy-3-methyl-but-2-enylpyrophosphate (HMBPP), or chemically similar molecules, wherein the phospho-antigens are naturally-occurring in cells as part of normal metabolic processes or the phospho-antigens are caused to accumulate in cells at higher levels due to treatment with bisphosphonate chemicals, wherein the activity of phospho-antigens is through direct interaction with the γδ TCR or wherein the activity of phospho-antigens is through interactions with butyrophilin (BTN) proteins BTN2A1, BTN3A1, BTN3A2, or BTN3A3.

16. The iPSC or T cell according to claim 12, wherein the rearranged γδ TCR is not activated by phospho-antigens.

17. The iPSC or T cell according to claim 12, comprising an exogenous polynucleotide encoding a human leukocyte antigen E (HLA-E) and/or human leukocyte antigen G (HLA-G).

18. The iPSC or T cell according to claim 12, wherein one or more of the exogenous polynucleotides are integrated at one or more loci on the chromosome of the cell selected from the group consisting of AAVS1, CCR5, ROSA26, collagen, HTRP, Hl 1, GAPDH, RUNX1, B2M, TAPI, TAP2, Tapasin, NLRC5, CIITA, RFXANK, CIITA, RFX5, RFXAP, TRAC, TRBC1, TRBC2, RAG1, RAG2, NKG2A, NKG2D, CD38, CIS, CBL-B, SOCS2, PD1, CTLA4, LAG3, TIM3, or TIGIT genes, provided at least one of the exogenous polynucleotides is integrated at a locus of a gene selected from the group consisting of B2M, TAP 1, TAP 2, Tapasin, RFXANK, CIITA, RFX5 and RFXAP genes to thereby result in a deletion or reduced expression of the gene.

19. The iPSC or T cell according to claim 18, wherein one or more of the exogenous polynucleotides are integrated at the loci of the CIITA, AAVS1 and B2M genes.

20. The iPSC or T cell according to claim 19 having a deletion or reduced expression of one or more of B2M or CIITA genes.

21. The iPSC or T cell according to claim 12, wherein one or more of the exogenous polynucleotides are integrated at a CD38 locus to thereby result in a deletion or reduced expression of the CD38 gene.

22. The iPSC or the T cell according to claim 1, wherein the CAR comprises:
(i) a signal peptide comprising a signal peptide;
(ii) an extracellular domain comprising a binding domain that specifically binds an antigen on a target cell;
(iii) a hinge region;
(iv) a transmembrane domain;
(v) an intracellular signaling domain; and
(vi) a co-stimulatory domain.

23. The iPSC or T cell according to claim 22, wherein the signal peptide is GMCSFR signal peptide.

24. The iPSC or T cell according to claim 22, wherein the extracellular domain comprises an scFv or V_{H}H derived from an antibody that specifically binds an antigen that is expressed on cancer cells.

25. The iPSC or T cell according to claim 22, wherein the hinge region comprises a CD28 hinge region, a CD8 hinge region, or an IgG hinge region.

26. The iPSC or T cell according to claim 22, wherein the transmembrane domain comprises a CD28 transmembrane domain or a CD8 transmembrane domain.

27. The iPSC or T cell according to claim 22, wherein the intracellular signaling domain is derived from DAP10, DAP12, Fc epsilon receptor I γ chain (FCER1G), FcR β, NKG2D, CD3δ, CD3ε, CD3γ, CD3ζ, CD5, CD22, CD226, CD66d, CD79A, or CD79B.

28. The iPSC or T cell according to claim 22, wherein the co-stimulatory domain is a co-stimulatory signaling domains are derived from CD28, 41BB, IL2Rb, CD40, OX40 (CD134), CD80, CD86, CD27, ICOS, NKG2D, DAP10, DAP12, or 2B4 (CD244).

29. The iPSC or T cell according to claim 22, wherein the CAR comprises:
(i) the signal peptide comprising an amino acid sequence having at least 90%, 91%, 92%, 93%, 94%, 95%, 96%, 97%, 98%, 99% or 100% sequence identity to SEQ ID NO: 1;
(ii) the extracellular domain comprising an amino acid sequence having at least 90%, 91%, 92%, 93%, 94%, 95%, 96%, 97%, 98%, 99% or 100% sequence identity to SEQ ID NO: 7;
(iii) the hinge region comprising an amino acid sequence having at least 90%, 91%, 92%, 93%, 94%, 95%, 96%, 97%, 98%, 99% or 100% sequence identity to SEQ ID NO: 22;
(iv) the transmembrane domain comprising an amino acid sequence having at least 90%, 91%, 92%, 93%, 94%, 95%, 96%, 97%, 98%, 99% or 100% sequence identity to SEQ ID NO: 24;
(v) the intracellular signaling domain comprising an amino acid sequence having at least 90%, 91%, 92%, 93%, 94%, 95%, 96%, 97%, 98%, 99% or 100% sequence identity to SEQ ID NO: 6; and
(vi) the co-stimulatory domain comprising an amino acid sequence having at least 90%, 91%, 92%, 93%, 94%, 95%, 96%, 97%, 98%, 99% or 100% sequence identity to SEQ ID NO: 20.

30. The iPSC or T cell according to claim 21, wherein the CAR comprises:
(i) the signal peptide comprising the amino acid sequence of SEQ ID NO: 1;
(ii) the extracellular domain comprising the amino acid sequence of SEQ ID NO: 7;
(iii) the hinge region comprising an amino acid sequence of SEQ ID NO: 22;
(iv) the transmembrane domain comprising the amino acid sequence of SEQ ID NO: 24;
(v) the intracellular signaling domain comprising the amino acid sequence of SEQ ID NO: 6; and
(vi) the co-stimulatory domain comprising the amino acid sequence of SEQ ID NO: 20.

31. The iPSC or T cell according to claim 12, wherein the mechanism of action of the artificial cell death polypeptide is metabolic, dimerization-inducing or therapeutic monoclonal antibody-mediated.

32. The iPSC or T cell according to claim 31, wherein the therapeutic monoclonal antibody mediated artificial cell death polypeptide is an inactivated cell surface protein selected from the group of monoclonal antibody specific epitopes selected from epitopes specifically recognized by ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, polatuzumab vedotin, ranibizumab, tocilizumab, trastuzumab, vedolizumab, adalimumab, belimumab, canakinumab, denosumab, golimumab, ipilimumab, ofatumumab, panitumumab, daratumumab, or ustekinumab.

33. The iPSC or T cell according to claim 32, wherein the inactivated cell surface protein is a truncated epithelial growth factor (tEGFR) variant.

34. The iPSC or T cell according to claim 33, wherein the tEGFR variant consists of an amino acid sequence having at least 90%, 91%, 92%, 93%, 94%, 95%, 96%, 97%, 98%, 99% or 100% sequence identity to SEQ ID NO: 71.

35. The iPSC or T cell according to claim 17, wherein the HLA-E comprises an amino acid sequence having at least 90%, 91%, 92%, 93%, 94%, 95%, 96%, 97%, 98%, 99% or 100% sequence identity to SEQ ID NO: 66 or the HLA-G comprises an amino acid sequence having at least 90%, 91%, 92%, 93%, 94%, 95%, 96%, 97%, 98%, 99% or 100% sequence identity to SEQ ID NO: 69.

36. The iPSC or the T cell according to claim 12, wherein:
(i) the exogenous polynucleotide encoding the chimeric antigen receptor (CAR) is integrated at a locus of AAVS1 gene;
(ii) the exogenous polypeptide encoding the artificial cell death polypeptide is integrated at a locus of CIITA gene; and
(iii) the exogenous polypeptide encoding the human leukocyte antigen E (HLA-E) and/or human leukocyte antigen G (HLA-G) is integrated at a locus of B2M gene;
wherein integration of the exogenous polynucleotides deletes or reduces expression of CIITA and B2M.

37. The iPSC or the T cell according to claim 12, wherein the CAR specifically binds CD38.

38. An induced pluripotent stem cell (iPSC) or a T cell comprising:
(i) an exogenous polynucleotide encoding a chimeric antigen receptor (CAR) having the amino acid sequence of SEQ ID NO: 61;
(ii) an exogenous polynucleotide encoding an artificial cell death polypeptide comprising an apoptosis-inducing domain having the amino acid sequence of SEQ ID NO: 71;
(iii) a polynucleotide encoding a rearranged T cell receptor (TCR) locus comprising a γ TCR having the amino acid sequence of SEQ ID NO: 133, 135, or 150 and a δ TCR having the amino acid sequence of SEQ ID NO: 134, 136, or 151; and
(iv) optionally, an exogenous polynucleotide encoding a human leukocyte antigen E (HLA-E) having the amino acid sequence of SEQ ID NO: 66;
wherein one or more of the exogenous polynucleotides are integrated at loci of AAVS1, CIITA and B2M genes, to thereby delete or reduce expression of CIITA and B2M.

39. A composition comprising the T cell according to claim 11.

40. The composition according to claim 39, further comprising or being used in combination with, one or more therapeutic agents selected from the group consisting of a peptide, a cytokine, a checkpoint inhibitor, a mitogen, a growth factor, a small RNA, a dsRNA (double stranded RNA), siRNA, oligonucleotide, mononuclear blood cells, a vector comprising one or more polynucleic acids of interest, an antibody, a chemotherapeutic agent or a radioactive moiety, or an immunomodulatory drug (IMiD).

41. The composition according to claim 40 comprising the antibody, wherein the antibody is an anti-CD20 antibody.

42. The composition according to claim 40 comprising the antibody, wherein the antibody comprises one or more selected from the group consisting of ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, polatuzumab vedotin, ranibizumab, tocilizumab, trastuzumab, vedolizumab, adalimumab, belimumab, canakinumab, denosumab, golimumab, ipilimumab, ofatumumab, panitumumab, obinutuzumab, daratumumab, or ustekinumab.

43. The composition according to claim 41, wherein the antibody comprises one or both of rituximab and obinutuzumab.

44. A method of treating cancer in a subject in need thereof, comprising administering the cell according to claim 1 to the subject in need thereof.

45. The method according to claim 44, wherein the cancer is non-Hodgkin's lymphoma (NHL) or multiple myeloma (MM).

46. The method according to claim 44, further comprising administering one or more antibodies selected from the group consisting of ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, polatuzumab vedotin, ranibizumab, tocilizumab, trastuzumab, vedolizumab, adalimumab, belimumab, canakinumab, denosumab, golimumab, ipilimumab, ofatumumab, panitumumab, obinutuzumab, daratumumab, or ustekinumab.

47. (canceled)

48. A method of manufacturing the T cell according to claim 11, comprising differentiating an iPSC cell according to claim 1 under conditions for cell differentiation to thereby obtain the T cell.

49.-50. (canceled)

51. A CD34+ hematopoietic progenitor cell (HPC) derived from an induced pluripotent stem cell (iPSC) comprising one or more polynucleotides encoding a rearranged γδ T cell receptor (TCR) and an exogenous polynucleotide encoding a chimeric antigen receptor (CAR); and one or more of:
(a) an exogenous polynucleotide encoding an artificial cell death polypeptide;
(b) a deletion or reduced expression of one or more of B2M, TAP 1, TAP 2, Tapasin, RFXANK, CIITA, RFX5 and RFXAP genes;
(c) a deletion or reduced expression of RAG1 and RAG2 genes;
(d) an exogenous polynucleotide encoding a non-naturally occurring variant of FcγRIII (CD16);
(e) an exogenous polynucleotide encoding interleukin 15 (IL-15) and/or interleukin (IL-15) receptor or a variant or truncation thereof;
(f) an exogeneous polynucleotide encoding a constitutively active interleukin 7 (IL-7) receptor or variant thereof;
(g) an exogenous polynucleotide encoding interleukin 12 (IL-12) or interleukin 21 (IL-21) or a variant thereof;
(h) an exogenous polynucleotide encoding human leukocyte antigen E (HLA-E) and/or human leukocyte antigen G (HLA-G);
(i) an exogenous polynucleotide encoding leukocyte surface antigen cluster of differentiation CD47 (CD47) and/or CD24; and
(j) an exogenous polynucleotide encoding one or more imaging or reporter proteins, such as PSMA or HSV-tk.

52.-55. (canceled)

56. An artificial cell death polypeptide comprising a herpes simplex virus thymidine kinase (HSV-tk) fused to a prostate-specific membrane antigen (PSMA) polypeptide via a linker.

57.-61. (canceled)

62. An artificial cell death polypeptide comprising a prostate-specific membrane antigen (PSMA) polypeptide and a cluster of differentiation 24 (CD24) polypeptide operably linked by an autoprotease peptide sequence.

63-64. (canceled)

65. An iPSC or T cell comprising an artificial cell death polypeptide of claim 56.

66. An iPSC or T cell comprising an artificial cell death polypeptide of claim 62.

67. An induced pluripotent stem cell (iPSC) or a T cell derived therefrom comprising: one or more polynucleotides encoding (a) a rearranged γδ T cell receptor (TCR) (b) an exogenous polynucleotide encoding a chimeric antigen receptor (CAR); and (c) an artificial cell death polypeptide comprising a herpes simplex virus thymidine kinase (HSV-tk) fused to a prostate-specific membrane antigen (PSMA) polypeptide via a linker.

68. A recombinant DLL4 variant polypeptide having an amino acid sequence selected from SEQ ID Nos 140-142.

69. A method of differentiating iPSC's to T cells comprising culturing cells in a medium comprising a recombinant Delta-like protein 4 (DLL4) variant selected from the polypeptides of claim 68.


SEQUENCE LISTING (155 sequences)

SEQ ID NO: 1 - protein, 22 aa, Homo sapiens
MLLLVTSLLLCELPHPAFLLIP

SEQ ID NO: 2 - protein, 120 aa, Artificial Sequence
  FMC63 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - protein, 18 aa, Artificial Sequence
  Whitlow Linker
GSTSGSGKPGSGEGSTKG

SEQ ID NO: 4 - protein, 107 aa, Artificial Sequence
  FMC63 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 5 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 6 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 245 aa, Artificial Sequence
  FMC63 scFV
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 8 - protein, 42 aa, Homo sapiens
KRGRKKLLYIFKQPFMRPVQTTQEEDGCSCRFPEEEEGGCEL

SEQ ID NO: 9 - protein, 94 aa, Homo sapiens
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 10 - protein, 62 aa, Homo sapiens
KKVAKKPTNKAPHPKQEPQEINFPDDLPGSNTAAPVQETLHGCQPVTQEDGKESRISVQERQ

SEQ ID NO: 11 - protein, 42 aa, Homo sapiens
ALYLLRRDQRLPPDAHKPPGGGSFRTPIQEEQADAHSTLAKI

SEQ ID NO: 12 - protein, 25 aa, Homo sapiens
TYCFAPRCRERRRNERLRRESVRPV

SEQ ID NO: 13 - protein, 61 aa, Homo sapiens
KWKKKKRPRNSYKCGTNTMEREESEQTKKREKIHIPERSDEAQRVFKSSKTSSCDKSDTCF

SEQ ID NO: 14 - protein, 48 aa, Homo sapiens
QRRKYRSNKGESPVEPAEPCHYSCPREEEGSTIPIQEDYRKPEPACSP

SEQ ID NO: 15 - protein, 38 aa, Homo sapiens
CWLTKKKYSSSVHDPNGEYMFMRAVNTAKKSRLTDVTL

SEQ ID NO: 16 - protein, 51 aa, Homo sapiens
MGWIRGRRSRHSWEMSEFHNYNLDLKKSDFSTRWQKQRCPVVKSKCRENAS

SEQ ID NO: 17 - protein, 24 aa, Homo sapiens
LCARPRRSPAQEDGKVYINMPGRG

SEQ ID NO: 18 - protein, 52 aa, Homo sapiens
YFLGRLVPRGRGAAEAATRKQRITETESPYQELQGQRSDVYSDLNTQRPYYK

SEQ ID NO: 19 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 20 - protein, 41 aa, Homo sapiens
RSKRSRLLHSDYMNMTPRRPGPTRKHYQPYAPPRDFAAYRS

SEQ ID NO: 21 - protein, 47 aa, Homo sapiens
TTTPAPRPPTPAPTIASQPLSLRPEACRPAAGGAVHTRGLDFACDIY

SEQ ID NO: 22 - protein, 39 aa, Homo sapiens
IEVMYPPPYLDNEKSNGTIIHVKGKHLCPSPLFPGPSKP

SEQ ID NO: 23 - protein, 21 aa, Homo sapiens
IYIWAPLAGTCGVLLLSLVIT

SEQ ID NO: 24 - protein, 27 aa, Homo sapiens
FWVLVVVGGVLACYSLLVTVAFIIFWV

SEQ ID NO: 25 - protein, 15 aa, Artificial Sequence
  (G4S)3
GGGGSGGGGSGGGGS

SEQ ID NO: 26 - protein, 20 aa, Artificial Sequence
  Linker 3
GGSEGKSSGSGSESKSTGGS

SEQ ID NO: 27 - protein, 8 aa, Artificial Sequence
  Linker 4
GGGSGGGS

SEQ ID NO: 28 - protein, 12 aa, Artificial Sequence
  Linker 5
GGGSGGGSGGGS

SEQ ID NO: 29 - protein, 16 aa, Artificial Sequence
  Linker 6
GGGSGGGSGGGSGGGS

SEQ ID NO: 30 - protein, 20 aa, Artificial Sequence
  Linker 7
GGGSGGGSGGGSGGGSGGGS

SEQ ID NO: 31 - protein, 20 aa, Artificial Sequence
  Linker 8
GGGGSGGGGSGGGGSGGGGS

SEQ ID NO: 32 - protein, 25 aa, Artificial Sequence
  Linker 9
GGGGSGGGGSGGGGSGGGGSGGGGS

SEQ ID NO: 33 - protein, 14 aa, Artificial Sequence
  Linker 10
IRPRAIGGSKPRVA

SEQ ID NO: 34 - protein, 15 aa, Artificial Sequence
  Linker 11
GKGGSGKGGSGKGGS

SEQ ID NO: 35 - protein, 15 aa, Artificial Sequence
  Linker 12
GGKGSGGKGSGGKGS

SEQ ID NO: 36 - protein, 15 aa, Artificial Sequence
  Linker 13
GGGKSGGGKSGGGKS

SEQ ID NO: 37 - protein, 15 aa, Artificial Sequence
  Linker 14
GKGKSGKGKSGKGKS

SEQ ID NO: 38 - protein, 15 aa, Artificial Sequence
  Linker 15
GGGKSGGKGSGKGGS

SEQ ID NO: 39 - protein, 15 aa, Artificial Sequence
  Linker 16
GKPGSGKPGSGKPGS

SEQ ID NO: 40 - protein, 20 aa, Artificial Sequence
  Linker 17
GKPGSGKPGSGKPGSGKPGS

SEQ ID NO: 41 - protein, 20 aa, Artificial Sequence
  Linker 18
GKGKSGKGKSGKGKSGKGKS

SEQ ID NO: 42 - protein, 14 aa, Artificial Sequence
  Linker 19
STAGDTHLGGEDFD

SEQ ID NO: 43 - protein, 15 aa, Artificial Sequence
  Linker 20
GEGGSGEGGSGEGGS

SEQ ID NO: 44 - protein, 15 aa, Artificial Sequence
  Linker 21
GGEGSGGEGSGGEGS

SEQ ID NO: 45 - protein, 15 aa, Artificial Sequence
  Linker 22
GEGESGEGESGEGES

SEQ ID NO: 46 - protein, 15 aa, Artificial Sequence
  Linker 23
GGGESGGEGSGEGGS

SEQ ID NO: 47 - protein, 20 aa, Artificial Sequence
  Linker 24
GEGESGEGESGEGESGEGES

SEQ ID NO: 48 - protein, 18 aa, Artificial Sequence
  Linker 25
GSTSGSGKPGSGEGSTKG

SEQ ID NO: 49 - protein, 19 aa, Artificial Sequence
  Linker 26
PRGASKSGSASQTGSAPGS

SEQ ID NO: 50 - protein, 19 aa, Artificial Sequence
  Linker 27
GTAAAGAGAAGGAAAGAAG

SEQ ID NO: 51 - protein, 19 aa, Artificial Sequence
  Linker 28
GTSGSSGSGSGGSGSGGGG

SEQ ID NO: 52 - protein, 20 aa, Artificial Sequence
  Linker 29
GKPGSGKPGSGKPGSGKPGS

SEQ ID NO: 53 - protein, 4 aa, Artificial Sequence
  Linker 30
GSGS

SEQ ID NO: 54 - protein, 10 aa, Artificial Sequence
  Linker 31
APAPAPAPAP

SEQ ID NO: 55 - protein, 20 aa, Artificial Sequence
  Linker 32
APAPAPAPAPAPAPAPAPAP

SEQ ID NO: 56 - protein, 32 aa, Artificial Sequence
  Linker 33
AEAAAKEAAAKEAAAAKEAAAAKEAAAAKAAA

SEQ ID NO: 57 - protein, 15 aa, Homo sapiens
EPKSCDKTHTCPPCP

SEQ ID NO: 58 - protein, 12 aa, Homo sapiens
ERKCCVECPPCP

SEQ ID NO: 59 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 60 - protein, 12 aa, Homo sapiens
ESKYGPPCPSCP

SEQ ID NO: 61 - protein, 486 aa, Artificial Sequence
  anti-CD19 scFv CAR
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - DNA, 1461 bp, Artificial Sequence
  anti-CD19 scFv CAR
atgctgctgctggtcacatctctgctgctgtgcgagctgccccatcctgcctttctgctgatccccgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaagatga

SEQ ID NO: 63 - DNA, 1724 bp, Artificial Sequence
  CAG Promoter
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggatatctacgaagcggccgccctctgctaaccatgttcatgccttcttctttttcctacagctcctgggcaacgtgctggttattgtgctgtctcatcattttggcaaa

SEQ ID NO: 64 - DNA, 121 bp, Artificial Sequence
  SV40 terminator/polyadenylation
aacttgtttattgcagcttataatggttacaaataaagcaatagcatcacaaatttcacaaataaagcatttttttcactgcattctagttgtggtttgtccaaactcatcaatgtatctt

SEQ ID NO: 65 - protein, 335 aa, Homo sapiens
HSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 66 - protein, 504 aa, Artificial Sequence
  HLA-G signal peptide-B2M-HLA-E
MVVMAPRTLFLLLSGALTLTETWAVMAPRTLILGGGGSGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 67 - DNA, 1515 bp, Artificial Sequence
  HLA-G signal peptide-B2M-HLA-E
atggtggtcatggcccctagaacactgttcctgctgctgtctggcgccctgacactgacagagacatgggccgtgatggcccccagaaccctgatcctgggcggcggtggttcaggcggaggaggttcaggaggagggggtagtggaggtggtggttctatccagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgacactgagccagcctaagatcgtgaagtgggatcgcgatatgggcggaggcggatctggtggcggaggaagtggcggcggaggatctggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaa

SEQ ID NO: 68 - protein, 312 aa, Homo sapiens
HSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 69 - protein, 471 aa, Artificial Sequence
  HLA-G signal peptide-B2M-HLA-G
MVVMAPRTLFLLLSGALTLTETWARIIPRHLQLGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 70 - DNA, 1422 bp, Artificial Sequence
  HLA-G signal peptide-B2M-HLA-G
gccaccatggtggtcatggcgccccgaaccctcttcctgctgctctcgggggccctgaccctgaccgagacctgggcgcggatcattccccgacatctgcaactgggaggcggcggttcaggagggggcggatcgatccaacgcacccccaagatccaggtctactccagacacccggccgaaaacggaaagtcgaacttcctgaactgctatgtgtcaggattccacccgtccgacatcgaggtggacctcctgaagaacggcgaacgcattgagaaggtcgagcactccgatctgtcgttctccaaggactggtccttctaccttctctactataccgaattcaccccgaccgagaaggacgaatacgcctgccgggtcaaccacgtgaccctgagccagccaaagatcgtgaaatgggaccgcgatatgggaggaggaggttccggcggaggaggaagcggaggcggaggttccggctcccactccatgaggtatttcagcgccgccgtgtcccggcctggccgcggagagcctcgcttcatcgccatgggatacgtggacgacacccagttcgtcagattcgacagcgacagcgcctgtcctcggatggaacctagagcaccttgggtcgagcaagagggccctgagtactgggaagaagagacacggaacaccaaggctcacgcccagaccgacagaatgaacctgcagaccctgcggggctactacaatcagtctgaggccagcagccatactctgcagtggatgatcggctgcgatctgggctctgatggcagactgctgagaggctacgagcagtacgcctacgacggcaaggattatctggccctgaacgaggacctgcggtcttggacagctgccgatacagccgctcagatcagcaagagaaagtgcgaggccgccaatgtggccgaacagagaagggcttacctggaaggcacctgtgtggaatggctgcacagatacctggaaaacggcaaagagatgctgcagcgggccgatcctcctaagacacatgtgacccaccatcctgtgttcgactacgaggccacactgagatgttgggccctgggcttttaccctgccgagatcatcctgacctggcagcgagatggcgaggatcagacccaggatgtggaactggtggaaaccagacctgccggcgacggcacctttcagaaatgggctgctgtggtggtgcccagcggagaggaacagagatacacctgtcacgtgcagcacgagggactgcctgaacctctgatgctgagatggaagcagagcagcctgcctacaatccccatcatgggaatcgtggccggactggtggttctggccgctgttgttacaggtgctgcagtggctgccgtgctgtggcggaagaaaagcagcgactga

SEQ ID NO: 71 - protein, 371 aa, Artificial Sequence
  truncated EGFR (tEGFR)
MRPSGTAGAALLALLAALCPASRAGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 72 - protein, 19 aa, Artificial Sequence
  P2A
ATNFSLLKQAGDVEENPGP

SEQ ID NO: 73 - protein, 19 aa, Artificial Sequence
  Signal Sequence
MEFGLSWVFLVALFRGVQC

SEQ ID NO: 74 - protein, 21 aa, Artificial Sequence
  Polivy epitope
ARSEDRYRNPKGSACSRIWQS

SEQ ID NO: 75 - protein, 10 aa, Artificial Sequence
  CD20 mimitope
ACPYANPSLC

SEQ ID NO: 76 - protein, 176 aa, Artificial Sequence
  ErbB epitope
EGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIGGGGSGG

SEQ ID NO: 77 - DNA, 1042 bp, Artificial Sequence
  B2M targeting left homology arm
gcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtc

SEQ ID NO: 78 - DNA, 1023 bp, Artificial Sequence
  B2M targeting right homology arm
atccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcag

SEQ ID NO: 79 - DNA, 7813 bp, Artificial Sequence
  B2M Targeting Plasmid
gcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcggcctccaacgcgtagatctattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggatatctacgaagcggccgccctctgctaaccatgttcatgccttcttctttttcctacagctcctgggcaacgtgctggttattgtgctgtctcatcattttggcaaagtcgacgccaccatggtggtcatggcccctagaacactgttcctgctgctgtctggcgccctgacactgacagagacatgggccgtgatggcccccagaaccctgatcctgggcggcggtggttcaggcggaggaggttcaggaggagggggtagtggaggtggtggttctatccagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgacactgagccagcctaagatcgtgaagtgggatcgcgatatgggcggaggcggatctggtggcggaggaagtggcggcggaggatctggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcagaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 80 - DNA, 1205 bp, Artificial Sequence
  AAVS1 targeting left homology arm
actctgccccaggcctccttaccattccccttcgacctactctcttccgcattggagtcgctttaactggccctggctttggcagcctgtgctgacccatgcagtcctccttaccatccctccctcgacttcccctcttccgatgttgagcccctccagccggtcctggactttgtctccttccctgccctgccctctcctgaacctgagccagctcccatagctcagtctggtctatctgcctggccctggccattgtcactttgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccct

SEQ ID NO: 81 - DNA, 1200 bp, Artificial Sequence
  AAVS1 targeting right homology arm
ccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgtacccctgcatcccccttcccctgcatcccccagaggccccaggccacctacttggcctggaccccacgagaggccaccccagccctgtctaccaggctgccttttgggtggattctcctccaactgtggggtgactgcttggcaaactcactcttcggggtatcccaggaggcctggagcattggggtgggctggggttcagagaggagggattcccttctcaggttacgtggccaagaagcaggggagctgggtttgggtcaggtctgggtgtggggtgaccagcttatgctgtttgcccaggacagcctag

SEQ ID NO: 82 - DNA, 7971 bp, Artificial Sequence
  AAVS1 Targeting plasmid
actctgccccaggcctccttaccattccccttcgacctactctcttccgcattggagtcgctttaactggccctggctttggcagcctgtgctgacccatgcagtcctccttaccatccctccctcgacttcccctcttccgatgttgagcccctccagccggtcctggactttgtctccttccctgccctgccctctcctgaacctgagccagctcccatagctcagtctggtctatctgcctggccctggccattgtcactttgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctgcggccgcacgcgtagatctattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggatatctacgaagcggccgccctctgctaaccatgttcatgccttcttctttttcctacagctcctgggcaacgtgctggttattgtgctgtctcatcattttggcaaagtcgacgccaccatgctgctgctggtcacatctctgctgctgtgcgagctgccccatcctgcctttctgctgatccccgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcacgacgactcctgctccaaggcctcctacacctgcaccaaccattgcaagtcagccgttgagcctccggccagaagcatgtcgcccagccgcaggcggggctgtacacacgagaggcttggatttcgcatgtgacatctatatctgggccccactggccggcacctgcggcgtgctgctgctgagcctggtgatcaccaagcgaggccgcaaaaaactcctttatatattcaagcaaccttttatgaggcccgtccagaccacgcaagaggaagatgggtgctcttgccgctttccagaggaagaggaggggggctgcgaacttagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataataaaacttgtttattgcagcttataatggttacaaataaagcaatagcatcacaaatttcacaaataaagcatttttttcactgcattctagttgtggtttgtccaaactcatcaatgtatcttaccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgtacccctgcatcccccttcccctgcatcccccagaggccccaggccacctacttggcctggaccccacgagaggccaccccagccctgtctaccaggctgccttttgggtggattctcctccaactgtggggtgactgcttggcaaactcactcttcggggtatcccaggaggcctggagcattggggtgggctggggttcagagaggagggattcccttctcaggttacgtggccaagaagcaggggagctgggtttgggtcaggtctgggtgtggggtgaccagcttatgctgtttgcccaggacagcctagaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 83 - DNA, 401 bp, Homo sapiens
cccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggaggtacaagagttgggtaaaaaaatcaaggtatttggtcccggaacaaagcttatcattacagataaacaacttgatgcagatgtttcccccaagcccactatttttcttccttcaattgctgaaacaaagctccagaaggctggaacatacctt

SEQ ID NO: 84 - DNA, 341 bp, Homo sapiens
ccaccctcaggtgctccatgaaaggagaagcgatcggtaactactatatcaactggtacaggaagacccaaggtagcacaatgactttcatataccaagaaaaggacatctatggccctggtttcaaagacaatttccaaggtgacattgatattgcaaagaacctggctgtacttaagatacttgcaccatcagagagagatgaagggtcttactactatgcctgtgacaccgttggggtcggtgtggtgtacaccgataaactcatctttggaaaaggaacccgtgtgactgtggaaccaagaagtcagcctcataccaaaccatccgtttttgtcatg

SEQ ID NO: 85 - DNA, 401 bp, Homo sapiens
cccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactaccgtgccttgtggagagaacaagagttgggcaaaaaaatcaaggtatttggtcccggaacaaagcttatcattacagataaacaacttgatgcagatgtttcccccaagcccactatttttcttccttcaattgctgaaacaaagctccagaaggctggaacatatctt

SEQ ID NO: 86 - DNA, 344 bp, Homo sapiens
ccaccctcaggtgctccatgaaaggagaagcgatcggtaactactatatcaactggtacaggaagacccaaggtaacacaatgactttcatataccgagaaaaggacatctatggccctggtttcaaagacaatttccaaggtgacattgatattgcaaagaacctggctgtacttaagatacttgcaccatcagagagagatgaagggtcttactaccgtgcctgtgacaccgtcgaaagaagtacccggacgtacaccgataaactcatctttggaaaaggaacccgtgtgactgtggaaccaagaagtcagcctcataccaaaccatccgtttttgtcatg

SEQ ID NO: 87 - DNA, 395 bp, Homo sapiens
cccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggtagtgttgggcaaaaaaatcaaggtatttggtcccgaaacaaagcttatcattacagataaacaacttgatgcaggtgtttcccccaagcccactatttttcttccttcaattgctgaaacaaagctccagaaggctggaacatacctt

SEQ ID NO: 88 - DNA, 350 bp, Homo sapiens
ccaccctcaggtgctccatgaaaggagaagcgatcggtaactactatatcaactggtacaggaagacccaaggtaacacaatgactttcatataccgagaaaaggacatctatggccctggtttcaaagacaatttccaaggtgacattgatattgcaaagaacctggctgtacttaagatacttgcaccatcagagagagatgaagggtcttactactgtgcctgtgacaccgtcagtcgggccggtactggggggcacaccaccgataaactcatctttagaaaaggaacccgtgtaactgtggaaccaagaagtcagcctcataccaaaccatccgtttttgtcatg

SEQ ID NO: 89 - DNA, 401 bp, Homo sapiens
cccgcctggagtgtgtggtgtctggaataacaatttctgcaacatctgtatattggtgtcgagagaggcctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactatgccttgtgggaggtcggggagttgggcaaaaaaatcaaggtatttggtcccggaacaaagcttatcattacagataaacaacttgatgcagatggttcccccaagcccactatttttcttccttcaattgctgaaacaaagctccagaaggctggagcatacctt

SEQ ID NO: 90 - DNA, 332 bp, Homo sapiens
ccaccctcaggtgctccatgaaaggagaagcgatcggtaactactatatcaactggtacaggaagacccaaggtaacacaatgactttcatataccgagaaaaggacatctatggccctggtttcaaagacaatttccaaggtgacattgatattgcaaagaacctggctgtacttaagatacttgcaccatcagagagagatgaagggtcttactactgtgcctgtgacaccgttaaaagggggacgcatgcactcatctttggaaaaagaacccgtgtgactgtggaaccaagaagtcagcctcataccaaaccatccgtttttgtcttg

SEQ ID NO: 91 - DNA, 398 bp, Homo sapiens
cccgcctggaatgtgtggtgtctggaataacaatttctgcaacatctgtatattggtatcgagagagacctggtgaagtcatacagttcctggtgtctatttcatatgacggcactgtcagaaaggaatccggcattccgtcaggcaaatttgagatggataggatacctgaaacgtctacatccactctcaccattcacaatgtagagaaacaggacatagctacctactaccgtgccttgtgggaggaagagttgggcaaaaaaatcaaggtatttggtcccggaacaaagcttatcattacagataaacagcttgatgcagatgtttcccccaagcccactatttttcttccttcaattgctgaaacaaagctccagaaggctggaacatacctt

SEQ ID NO: 92 - DNA, 329 bp, Homo sapiens
ccaccctcaggtgctccatgaaaggagaagcgatcggtaactactatatcaactggtacaggaagacccaaggtaacacaatgactttcatataccgagaaaaggacatctatggccctggtttcaaagacaatttccaaggtgacattgatattgcaaagaacctggctgtacttaagatacttgcaccatcagagagagatgaagggtcttactactgtgcctgtgacatactgggggacaccgataaactcatctttgaaaaaggaacccgtgtgactgtggaaccaagaagtcagcctcataccaaaccatccgtttttgtcatg

SEQ ID NO: 93 - DNA, 401 bp, Homo sapiens
cccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgagccttgtgggaggtgcgggagttgggcaaaaaaatcaaggtatttggtcccggaacaaagcttatcattacagataaacaacttgatgcagatgtttcccccaagcccactatttttcttccttcaattgctgaaacaaagctccagaaggctggaacatatcct

SEQ ID NO: 94 - DNA, 338 bp, Homo sapiens
ccaccctcaggtgctccatgaaaggagaagcgatcggtaactactatatcaactggtacaggaagacccaaggtaacacaatgactctcatataccgggaaaaggacatctatggccctggtttcaaagacaatttccaaggtgacattgatattgcaaagaacctggctgtacttaagatacttgcaccatcagagagagatgaagggtcttactaccgtgcctgtgactctgtactgggggatttgtacaccgataaactcatctttggaaaaggagcccgtgtgactgtggaaccaagaagtcagcctcacaccaaaccatccgtttttgtcatg

SEQ ID NO: 95 - DNA, 401 bp, Homo sapiens
cccgcctggaatgtgtggtgtctggaataacaatttctgcaacatctgtatattggtatcgagagaggcctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggaggtcggggagttgggcaaaaaaatcaaggtatttggtcccgaaacaaagcttatcattacagataaacaacttgatgcagatgtttcccccaagcccactatttttcttccttcaattgctgaaacaaagctccagaaggctggaacatacctt

SEQ ID NO: 96 - DNA, 332 bp, Homo sapiens
ccaccctcaggtgctccatgaaaggagaagcgatcggtaactactatatcaactggtacaggaagacccaaggtaacacaatgactttcatataccgagaaaaggacatctatggccctggtttcaaagacaatttccaaggtgacattgatattgcaaagaacctggctgtacttaagatacttgcaccatcagagagagatgaagggtcttactactgagcctgtgacaccgttaaaagggggacgcatgcactcatctttggaaaaggaacccgtgtgactgtggaaccaagaagtcagcctcataacaaaccatccgtttttgtcatg

SEQ ID NO: 97 - DNA, 402 bp, Homo sapiens
cccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactaccgtgccttgtgggaggtgttccaggagttgggcaaaaaaatcaaggtatttggtcccggaacaaagcttatcattacagataaacaacttgatgcagatgtttcccaagcccactatttttcttccttcaattgctgaaacaaagctccagaaggctggaacatacctt

SEQ ID NO: 98 - DNA, 334 bp, Homo sapiens
ccaccctcaggtgctccatgaaaggagaagcgatcggtaactactatatcaactggtacaggaagacccaaggtaacacaatgactttcatataccgagaaaaggacatctatggccctggtttcaaagacaatttccaaggtgacattgatattgcaaagaacctggctgtacttaagatacttgcaccatcagagagagatgaagggtcttactactatgcctgcgacaccctaggtatgccgggggatacggacaccgataaactcatctttggaaaaggaacccgtgtgactgtggaaccaagaagtcagcctcataccaaaccatccgt

SEQ ID NO: 99 - DNA, 401 bp, Homo sapiens
cccgcctggaatgtgtggtgtctggaataacaatttctgcaacatctgtatattggtatcgagagagacctggtggagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactaccgtgccttgtgggaggtgttcgggttgggcaaaaaaatcaaggtatttggtcccggaacaaagcttatcattacagataaacaacttgatgcagatgtttcccccaagcccactatttttcttccttcaattgctgagacaaagctccagaaggctggaacatatctt

SEQ ID NO: 100 - DNA, 335 bp, Homo sapiens
ccaccctcaggtgctccatgaaaggagaagcgatcggtaactactatatcaactggtacaggaagacccaaggtaacacaatgactttcatataccgagaaaaggacatctatggccctggtttcaaagacaatttccgaggtgacattgatattgcaaagaacctggctgtacttaagatacttgcaccatcagagagagatgaagggtcttactactgtgcctgtgacgacctgaggggaagggataccgataaactcatctttagaaaaggaacccgtgtgactgtggaaccaagaagtcagcctcataccaaaccatccgtttttgtcatg

SEQ ID NO: 101 - DNA, 398 bp, Homo sapiens
cccgcctggaatgcgtggtgtctggaataacaatttctgcaacatctgtatattgagagagacctggtgaagtcatacagttcctggtgtccatttcatatgacggcactgtcagaaaggaatccggcatcccgtcaggcaaatttgaggtggataggatacctgaaacgtctacatccactctcaccattcacaatgtagagaaacaggacatagctacctactactgtgccttgtgggaggtgttccaggagttgggcaaaaaaatcaaggtatttgatcccggaacaaagcttatcattacagataaacaacttgatgcagatgtttcccccaagcccactatttttcttccttcaattgctgaaacaaagctccagaaggctggaacatacctt

SEQ ID NO: 102 - DNA, 344 bp, Homo sapiens
ccaccctcaggtgctccatgaaaggagaagcgatcggtaactactatatcaactggtacaggaagacccaaggtaacacaatgactttcatataccgagaaaaggacatctatggccctggtttcaaagacaatttccaaggcgacattgatattgcaaagaacctggctgtacttaagatacttgcaccatcagagagagatgaagggtcttactacggtgcctgtgacaccctaggtatgccgggggatacggacaccgataaactcatctttggaaaaggaacccgtgtgactgtggaaccaagaagtcagcctcataccaaaccatccgtttttgtcatg

SEQ ID NO: 103 - DNA, 404 bp, Homo sapiens
cccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggaggtgcttccaaagttgggcaaaaaaatcaaggtatttagtcccggaacaaagcttatcattacagataaacaacttgatgcagatgtttcccccaagcccactatttttcttccttcaattgctggaacaaagctccagaaggctggaacatacctt

SEQ ID NO: 104 - DNA, 338 bp, Homo sapiens
ccaccctcaggtgctccatgaaaggagaagcgatcggtaactactatatcaactggtacaggaagacccaaggtaacacaatgactttcatataccgagaaaaggacatctatggccctggtttcaaagacaatttccaaggtgacattgatattgcaaagaacctggctgtacttaagatacttgcaccatcagagagagatgaagggtcttactaccgtgcctgtgacatgctactgggggataccgacaccgataaactcatctttggaaaaggaacccgtgtgactgtggaaccaagaagtcagcctcataccaaaccatccgtttttgtcatg

SEQ ID NO: 105 - DNA, 401 bp, Homo sapiens
cccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggatcttggcgagttgggcaaaaaaatcaaggtatttgatcccggaacaaagcttgtcattacagataaacaacttgatgcagatgtttcccccaagcccactatttttcttccttcaattgctgaaacaaagctccagaaggctggaacatacctt

SEQ ID NO: 106 - DNA, 338 bp, Homo sapiens
ccaccctcaggtgctccatgaaaggagaagcgatcggtaactactatatcaactggtacaggaagacccaaggtaacacaatgactttcatataccgagaaaaggacatctatggccctggtttcaaagacaatttccaaggtgacattgatattgcaaagaacctggctgtacttaagatacttgcaccatcagagagagatgaagggtcttactaccgtgcctgtgactcactgggggatagtggatacaccgataaactcatctttggaaaaggaacccgtgtgactgtggaaccaagaagtcagcctcataccaaaccatccgtttttgtcatg

SEQ ID NO: 107 - DNA, 386 bp, Homo sapiens
cccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagagacaggacatagctacctactaccgtgccttgtgggagctcagaaagaaactctttggcagtggaacaacactggttgtcacagataaacaacttgatgcagatgtttctcccaagcccactatttttcttccttcaattgctgaaacaaagctccagaaggctggaacatatctt

SEQ ID NO: 108 - DNA, 335 bp, Homo sapiens
ccaccctcaggtgctccatgaaaggagaagcgatcggtaactactatatcaactggtacaggaagacccaaggtaacacaatgactttcatataccgagaaaaggacatctatggccctggtttcaaagacaatttccaaggtgacattgatattgcaaagaacctggctgtacttaagatacttgcaccatcagagagagatgaagagtcttactaccgtgcctgtgacttcgtactgggggcgatgtccgataaactcatctttggaaaaggaacccgtgtgactgtggaaccaagaagtcagcctcataccaaaccatccgtttttgtcatg

SEQ ID NO: 109 - protein, 14 aa, Homo sapiens
ALWEVQELGKKIKV

SEQ ID NO: 110 - protein, 16 aa, Homo sapiens
ACDTVGVGVVYTDKLI

SEQ ID NO: 111 - protein, 14 aa, Homo sapiens
ALWREQELGKKIKV

SEQ ID NO: 112 - protein, 17 aa, Homo sapiens
ACDTVERSTRTYTDKLI

SEQ ID NO: 113 - protein, 12 aa, Homo sapiens
ALWVVLGKKIKV

SEQ ID NO: 114 - protein, 19 aa, Homo sapiens
ACDTVSRAGTGGHTTDKLI

SEQ ID NO: 115 - protein, 14 aa, Homo sapiens
ALWEVGELGKKIKV

SEQ ID NO: 116 - protein, 13 aa, Homo sapiens
ACDTVKRGTHALI

SEQ ID NO: 117 - protein, 13 aa, Homo sapiens
ALWEEELGKKIKV

SEQ ID NO: 118 - protein, 12 aa, Homo sapiens
ACDILGDTDKLI

SEQ ID NO: 119 - protein, 14 aa, Homo sapiens
ALWEVRELGKKIKV

SEQ ID NO: 120 - protein, 15 aa, Homo sapiens
ACDSVLGDLYTDKLI

SEQ ID NO: 121 - protein, 14 aa, Homo sapiens
ALWEVGELGKKIKV

SEQ ID NO: 122 - protein, 13 aa, Homo sapiens
ACDTVKRGTHALI

SEQ ID NO: 123 - protein, 15 aa, Homo sapiens
ALWEVFQELGKKIKV

SEQ ID NO: 124 - protein, 17 aa, Homo sapiens
ACDTLGMPGDTDTDKLI

SEQ ID NO: 125 - protein, 14 aa, Homo sapiens
ALWEVFGLGKKIKV

SEQ ID NO: 126 - protein, 14 aa, Homo sapiens
ACDDLRGRDTDKLI

SEQ ID NO: 127 - protein, 15 aa, Homo sapiens
ALWEVLPKLGKKIKV

SEQ ID NO: 128 - protein, 15 aa, Homo sapiens
ACDMLLGDTDTDKLI

SEQ ID NO: 129 - protein, 14 aa, Homo sapiens
ALWDLGELGKKIKV

SEQ ID NO: 130 - protein, 15 aa, Homo sapiens
ACDSLGDSGYTDKLI

SEQ ID NO: 131 - protein, 9 aa, Homo sapiens
ALWELRKKL

SEQ ID NO: 132 - protein, 14 aa, Homo sapiens
ACDFVLGAMSDKLI

SEQ ID NO: 133 - protein, 314 aa, Homo sapiens
MLSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEVGELGKKIKVFGPGTKLIITDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 134 - protein, 289 aa, Homo sapiens
MQRISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDTVKRGTHALIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 135 - protein, 315 aa, Homo sapiens
MLSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEVFQELGKKIKVFGPGTKLIITDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 136 - protein, 293 aa, Homo sapiens
MQRISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDTLGMPGDTDTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 137 - protein, 601 aa, Artificial Sequence
  DLL4-Fc Fusion 1
SGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 484 aa, Artificial Sequence
  DLL4-Fc Fusion 2
SGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 725 aa, Artificial Sequence
  DLL4-Fc Fusion 3
SGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 484 aa, Artificial Sequence
  DLL4-Fc Fusion 4
SSVFQLQLQEFINERGVLASGRPCEPGCRTFFRVCLKHFQAVVSPGPCTFGTVSTPVLGTNSFAVRDDSSGGGRNPLQLPLNFTWPGTFSLIIEAWHAPGDDLRPEALPPDALISKFAIQGSLAVGQNWLLDEQTSTLTRLRYSYRVICSDNYYGDNCSRLCKKRNDYFGHYVCQPDGNPSCLPGWTGEYCQQPICLSGCHEQNGYCSKPAECLCRPGWQGRLCNECIPHNGCRHGTCSTPWQCTCDEGWGGLFCDQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 484 aa, Artificial Sequence; identical to SEQ ID NO: 140
  DLL4-Fc Fusion 5

SEQ ID NO: 142 - protein, 725 aa, Artificial Sequence
  DLL4-Fc Fusion 6
SSVFQLQLQEFINERGVLASGRPCEPGCRTFFRVCLKHFQAVVSPGPCTFGTVSTPVLGTNSFAVRDDSSGGGRNPLQLPLNFTWPGTFSLIIEAWHAPGDDLRPEALPPDALISKFAIQGSLAVGQNWLLDEQTSTLTRLRYSYRVICSDNYYGDNCSRLCKKRNDYFGHYVCQPDGNPSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 376 aa, Artificial Sequence
  Thymidine kinase
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 144 - protein, 741 aa, Artificial Sequence
  truncated PSMA
MWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 145 - protein, 1135 aa, Artificial Sequence
  TK-PSMA fusion
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANGSTSGSGKPGSGEGSTKGMWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 146 - protein, 1139 aa, Artificial Sequence
  TK-T2A-PSMA
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANSGSGEGRGSLLTCGDVEENPGPMWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 147 - protein, 843 aa, Artificial Sequence
  PSMA-T2A-CD24
MWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVASGSGEGRGSLLTCGDVEENPGPMGRAMVARLGLGLLLLALLLPTQIYSSETTTGTSSNSSQSTSNSGLAPNPTNATTKAAGGALQSTASLFVVSLSLLHLYS

SEQ ID NO: 148 - DNA, 945 bp, Artificial Sequence
  Gamma chain TCR sequence
atgctgtctctgctgcacgcctctacactggctgttctgggagccctgtgtgtgtatggcgccggacatctggaacagccccagatcagcagcaccaagacactgagcaagaccgccagactggaatgcgtggtgtccggcatcacaatcagcgccacaagcgtgtactggtacagagaaagacccggcgaagtgatccagttcctggtgtccatcagctacgatggcaccgtgcggaaagagagcggcatcccttctggcaagttcgaggtggacagaatccccgagacaagcaccagcacactgaccatccacaacgtggaaaagcaggatatcgccacctactactgcgccctgtgggaagtgcaagagctgggcaagaaaatcaaggtgttcggccctggcaccaagctgatcatcaccgacaaacagctggacgccgacgtgtcccctaagcctaccatcttcctgccttctatcgccgagacaaagctgcagaaggccggcacctatctgtgcctgctggaaaagttcttcccagacgtgatcaagatccactggcaagagaagaagtccaacaccatcctgggcagccaagagggcaacaccatgaagaccaacgacacctacatgaagttcagctggctgaccgtgcctgagaagtccctggacaaagaacaccggtgcatcgtgcggcacgagaacaacaagaacggcgtggaccaagagatcatcttcccacctatcaagaccgacgtcatcaccatggatcccaaggacaactgcagcaaggacgccaacgataccctgctgctgcagctgacaaacaccagcgcctactacatgtatttgctgctgttgctgaagtccgtggtgtacttcgccatcatcacctgttgcctgctgcggagaaccgccttctgctgcaatggcgagaagtcctga

SEQ ID NO: 149 - DNA, 867 bp, Artificial Sequence
  Delta chain TCR sequence
atgcagagaatcagcagcctgatccacctgagcctgttctgggctggcgtgatgtctgccattgagctggtgcctgagcaccagaccgtgcctgtgtctattggagtgcccgccacactgcggtgtagcatgaagggcgaagccatcggcaactactacatcaactggtacagaaagacccagggcaacaccatgaccttcatctacagagagaaggacatctacggccctggcttcaaggacaacttccagggcgacatcgacattgccaagaacctggccgtgctgaagattctggcccctagcgagagagatgagggcagctactactgcgcctgtgacaccgtgggcgacaccgataagctgatcttcggcaagggcaccagagtgaccgtggaacctagaagccagcctcacaccaagcctagcgtgttcgtgatgaagaacggcaccaacgtggcctgcctggtcaaagagttctaccccaaggacatccggatcaacctggtgtccagcaagaagatcaccgagttcgaccccgccatcgtgatcagccctagcggcaagtacaacgccgtgaagctggggaagtacgaggacagcaatagcgtgacctgcagcgtgcagcacgacaacaagaccgtgcacagcaccgatttcgaagtgaaaaccgactccaccgaccacgtgaagcccaaagagacagagaacaccaagcagcccagcaagagctgccacaagcctaaggccatcgtgcacaccgagaaagtgaacatgatgagcctgaccgtgctgggcctgagaatgctgttcgccaaaacagtggccgtgaacttcctgctgaccgccaagctgttcttcctgtga

SEQ ID NO: 150 - protein, 314 aa, Artificial Sequence
  Gamme chain TCR sequence
MLSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEVQELGKKIKVFGPGTKLIITDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 151 - protein, 288 aa, Artificial Sequence
  Delta chain TCR sequence
MQRISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDTVGDTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 152 - protein, 15 aa, Artificial Sequence
  Gamma chain TCR gene CDR3 sequence
ALWEVQELGKKIKVF

SEQ ID NO: 153 - protein, 12 aa, Artificial Sequence
  Delta chain TCR gene CDR3 sequence
ACDTVGDTDKLI

SEQ ID NO: 154 - protein, 17 aa, Artificial Sequence
  Gamma chain TCR gene CDR3 sequence
ALWEGTPGELGKKIKVF

SEQ ID NO: 155 - protein, 14 aa, Artificial Sequence
  Delta chain TCR gene CDR3 sequence
ACDRLGDTRTKLIF